(19) United States
(12) Patent Application Publication
(10) Pub. No.: US 2023/0019098 A1
(43) Pub. Date: Jan. 19, 2023

(54) BIVALENT COMPOUNDS, CONJUGATES AND USES THEREOF

(71) Applicant: RISEN (SUZHOU) PHARMA TECH CO., LTD., Suzhou (CN)

(72) Inventors: Jiasheng LV, Suzhou (CN); Haixiao SIYANG, Suzhou (CN); Yijie YIN, Suzhou (CN); Wantao GUO, Suzhou (CN); Haiming LI, Suzhou (CN); Dawei CHEN, Suzhou (CN); Jiamin GU, Suzhou (CN); Xianqi KONG, Dollard-des-Ormeaux (CA); Jun PAN, Suzhou (CN); Xinxin MA, Suzhou (CN); Peiming SONG, Suzhou (CN); Chun WU, Suzhou (CN); Hui FENG, Suzhou (CN); Sheng YAO, Suzhou (CN)

(21) Appl. No.: 17/844,407

(22) Filed: Jun. 20, 2022

(30) Foreign Application Priority Data
Jun. 21, 2021 (CN) 202110687927.7

(51) Int. Cl. A61K 47/54; C07C 275/16

(52) U.S. Cl. CPC A61K 47/542; C07C 275/16; A61K 47/545; A61K 47/549

(57) ABSTRACT

There are provided bivalent compounds and conjugates thereof, the conjugates comprising a bivalent compound, a targeting moiety, and a bioactive agent, as well as pharmaceutical compositions and methods of use of the conjugate for the treatment, inhibition, or prevention of diseases and disorders which are therapeutic targets of the bioactive agent.

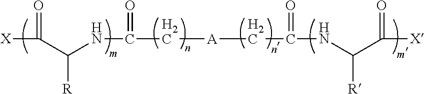

### CROSS-REFERENCE TO RELATED APPLICATIONS

This application claims the benefit of priority from Chinese application no. 202110687927.7, filed Jun. 21, 2021, the entire contents of which are hereby incorporated by reference.

### FIELD

The present disclosure relates to bivalent compounds and conjugates comprising the bivalent compound, a targeting moiety, and a bioactive agent, as well as pharmaceutical compositions and methods of use thereof for the prevention, treatment, and inhibition of diseases and disorders which are therapeutic targets of the bioactive agent.

### BACKGROUND

Oligonucleotides are generally composed of fewer than 20 single-stranded nucleotides (deoxyribonucleotides or ribonucleotides). They can pair with deoxyribonucleic acid (DNA), messenger ribonucleic acid (mRNA) or precursor messenger ribonucleic acid (pre-mRNA) via Watson-Crick base complementary pairing with high specificity. Due to the high specificity of base pairing, oligonucleotides can be used to inhibit or silence target genes with great specificity. For example, they can be used to inhibit or silence disease-causing mutated genes that encode aberrant proteins or are otherwise involved in etiology of disease. Many kinds of oligonucleotides are used therapeutically or are in clinical development, including for example antisense oligonucleotides (ASOs), small interfering RNAs (siRNAs), microRNAs (miRNAs), aptamers, and the like.

siRNA compounds are promising agents for the diagnosis and treatment of a variety of diseases and can also be used to elucidate gene function. For example, siRNA compounds offer great potential as novel therapeutic agents for silencing pathogenic genes. Currently, potential siRNA therapeutics are being investigated for the treatment of many diseases, including central nervous system diseases, inflammatory diseases, metabolic disorders, oncology indications, infectious diseases, eye diseases, etc. More than 20 candidate RNA interference (RNAi)-based therapeutics are currently in clinical trials, and positive results from these trials will support further development of clinically relevant RNAi therapies (Bobbin & Rossi, Annu. Rev. Pharmacol. Toxicol., 2016, 56:103-22). These new therapeutics in development include siRNA conjugates for the treatment of diseases (Weingaertner & Bethge (2019), WO2019193144; Bethge et al. (2019), WO2019193189; Zhang et al. (2019), WO2019105437; Zhang et al. (2019), WO2019105414; Nair et al. (2019), WO2019217459; Nair, et al. (2015), US2015/0196655; Muthiah et al. (2015), WO2015006740).

Synthetic small interfering RNAs (siRNAs) have been shown to inhibit expression of disease-causing genes through post-transcriptional gene silencing mediated by the endogenous RNA interference (RNAi) pathway. However, efficient delivery of siRNA therapeutics to target cells or organs remains a challenge and has proven to be an enormous obstacle to the successful development of such therapeutics. Efficient delivery of siRNA has been achieved by covalently linking various elements that can promote intracellular absorption, target specific cells or tissues, or reduce drug clearance in the circulation. These elements include lipids (e.g., cholesterol, which promotes interaction with circulating lipoprotein particles), peptides (e.g., for cell targeting and/or cell penetration), aptamers, antibodies, and sugars (e.g., N-acetylgalactosamine (GalNAc)). For example, U.S. Pat. No. 8,828,956 describes carbohydrate conjugates as delivery agents for oligonucleotides. Carbohydrate conjugates and iRNA agents comprising the conjugates, which are advantageous for the in vivo delivery of the iRNA agents, as well as iRNA compositions suitable for in vivo therapeutic use are described, as well as methods of making these compositions and methods of introducing these iRNA agents into cells using these compositions, e.g., for the treatment of various disease conditions. In particular, the carbohydrate-coupled siRNA reagents target the parenchymal cells of the liver.

N-acetylgalactosamine (GalNAc) is a high affinity ligand of the asialoglycoprotein receptor (ASGPR), which is specifically expressed in hepatocytes and located on their surface. Binding of GalNAc to the ASGPR triggers clathrin-mediated endocytosis to enable intracellular delivery of siRNA and elicit RNAi-mediated RNA silencing in the liver (Nair, J. K. et al. Nucleic Acids Research, 2017, 45(19), 10969-10977). GalNAc has been used in recent years as a targeting molecule for targeted delivery of nucleic acid drugs to the liver. For example, Alnylam Pharmaceuticals, Inc. reported for the first time that an siRNA based on GalNAc coupling technology could exert interference activity in mice (Nair, J. Am. Chem. Soc., 2014, 136, 16958-16961). This paper reported that an siRNA conjugated to a trivalent GalNAc ligand demonstrated targeted delivery to the liver and RNAi-mediated RNA silencing activity both in vivo and in vitro, with the ED₅₀ of a single dose determined to be 1 mg/kg, and the single injection dose less than 1 mL. In longer term experiments, subcutaneous injection once a week gave up to 9 months of stable interference activity.

More than 10 kinds of oligonucleotide therapeutics have been approved by the FDA and other regulatory agencies, for a variety of indications. For example, Inclisiran (sold under the brand name Leqvio®, owned by Novartis), was approved by the European Commission in December 2020 for the treatment of adult primary hypercholesterolemia (including heterozygous familial and non-familial hypercholesterolemia) or mixed dyslipidemia as an adjunctive diet therapy. Inclisiran is used for the treatment of people with atherosclerotic cardiovascular disease (ASCVD), ASCVD risk equivalents, hypercholesterolemia, and dyslipidemia (Khvorova, A., 2017, N Engl J Med 376: 4-7; Fitzgerald, K. et al., 2017, N Engl J Med 376: 41-51; U.S. Pat. No. 8,828,956; WO2014/089313). Inclisiran is a chemically modified siRNA conjugated to triantennary GalNAc that targets the 3′ UTR of proprotein convertase subtilisin-kexin type 9 (PCSK9) and inhibits synthesis of the PCSK9 enzyme. Down-regulation of PCSK9, a protein involved in low-density lipoprotein (LDL) receptor degradation, results in the up-regulation of LDL receptor levels on the surface of the hepatocytes, supporting more efficient clearance of LDL cholesterol from the bloodstream. International PCT Application Publication No. WO2014/089313 describes PCSK9 iRNA compositions and methods of use thereof. Double-stranded RNAi agents targeting the PCSK9 and methods of using such RNAi agents to inhibit expression of PCSK9 and methods of treating subjects having a lipid disorder, such as a hyperlipidemia, are described.

However, one of the main obstacles to the wide application of oligonucleotide therapies is the difficulty of their effective delivery to target organs or tissues. In order to improve the transmembrane delivery of nucleic acids and oligonucleotides, many delivery strategies have been used, including protein carriers, antibody carriers, liposome delivery systems, electroporation, direct injection, cell fusion, viral vectors and calcium phosphate-mediated transformation. At present, many of these technologies are limited by the types of cells that allow transmembrane transport and the conditions required to achieve this transport. Improving the efficiency of in vivo delivery of oligonucleotide therapeutic agents remains an urgent problem.

### SUMMARY

It is an object of the present invention to provide compounds which improve the targeted delivery of therapeutic agents, as well as therapeutic and prophylactic agents comprising the new compounds described herein, pharmaceutical compositions thereof, and therapeutic and prophylactic methods of use thereof.

The present invention is based, at least in part, on technical research into coupling and combining targeting moieties (such as biological ligands such as carbohydrates, polypeptides or lipophiles) with bioactive agents (such as therapeutic oligonucleotides such as siRNAs) by using novel bivalent compounds which can be conjugated bidirectionally, thereby generating new conjugated compounds. The conjugates serve to optimize the targeted delivery of the bioactive agents in vivo. Thus, the main technical problem to be solved by the disclosure is provision of a bivalent compound which effectively connects or couples the targeting moiety with the bioactive agent, so as to generate a conjugated compound which delivers the bioactive agent effectively to target cells, organs and tissues. In this way the bivalent compounds and conjugates can achieve enhanced delivery efficiency for the relevant bioactive agent and/or increases its therapeutic effect.

In a first broad aspect, there are provided bivalent compounds, and pharmaceutically acceptable salts or esters thereof, capable of being conjugated directly or indirectly to both a targeting moiety and a bioactive agent, to form a corresponding conjugate. In some embodiments, the bivalent compound is a compound of Formula (I):

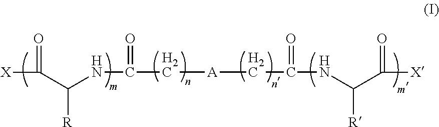

wherein: X and X′ are independently selected from a hydroxyl group (OH), an amino group (NH₂), and a halide; R and R′ are independently a side chain of a natural or unnatural amino acid, and R and R′ may be the same or different; m and m′ are independently an integer from 0 to 3, provided that when m or m′ is 1, one or both of the structural fragment

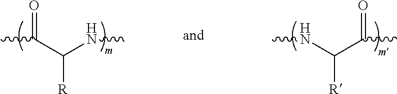

is a natural or unnatural amino acid residue and the structural fragments may be the same or different; provided that when m or m′ is 2 or 3, one or both of the structural fragment

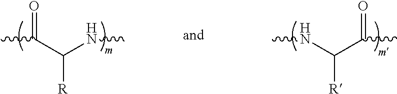

is a residue of a dipeptide or tripeptide and the structural fragments may comprise the same or different amino acid residues; n and n′ are independently an integer from 0 to 10, provided that n and n′ are not both 0; and A is selected from

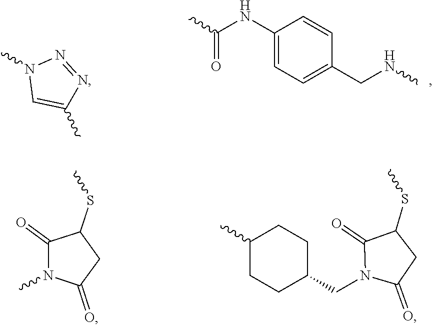

and a methylene (CH₂), provided when A is a methylene, m and m′ are not both 0.

In accordance with the disclosure, bivalent compounds have active functional groups at both ends. The bivalent compounds are directly or indirectly coupled with the targeting moiety and the bioactive agent through the active functional groups. In this way, the bivalent compounds can effectively combine the targeting moiety and the bioactive agent into a corresponding conjugated compound (a “conjugate”) and thereby improve the delivery efficiency of the bioactive agent.

In some embodiments of Formula (I), A is a methylene, and the bivalent compound is a compound of Formula (II), or a pharmaceutically acceptable salt or ester thereof:

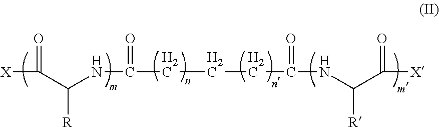

where X, X′, R, R′, m, m′, n, and n′ are as defined above.

In some embodiments of Formula (I) and Formula (II), the amino acid is independently selected from citrulline, homocitrulline, lysine, homolysine, asparagine, glutamine, arginine, glycine, methionine, phenylalanine, albizziin, valine, and combinations thereof.

In some embodiments, the bivalent compound is a compound of Formula(III) or Formula (IV):

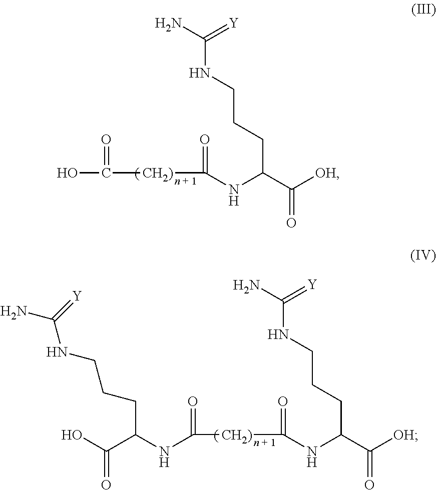

where Y is selected from oxygen (O) and nitrogen (NH) and n is as defined above. In one embodiment, when Y is oxygen, the amino acid residues in the molecule are derived from citrulline. In another embodiment, when Y is nitrogen, the amino acid residues in the molecule are derived from arginine.

In some embodiments of bivalent compounds of the disclosure, the amino acid residues are derived from natural amino acids, unnatural amino acids, and any combination thereof. In some embodiments, the amino acid residues are derived from natural amino acids. In some embodiments, the amino acid residues are derived from unnatural amino acids. In some embodiments, the amino acid residues are derived from a combination of natural and unnatural amino acids.

In some embodiments of bivalent compounds of the disclosure, the amino acid residues are derived from L-amino acids, D-amino acids, dl-amino acids, and any combination thereof. In some embodiments, the amino acid residues are derived from L-amino acids. In some embodiments, the amino acid residues are derived from D-amino acids. In some embodiments, the amino acid residues are derived from dl-amino acids. In some embodiments, the amino acid residues are derived from a combination of L-, D-, and/or dl- amino acids.

In one embodiment, the amino acid residues in bivalent compounds of the disclosure are L-amino acids. In one embodiment, the amino acid residues in bivalent compounds of the disclosure are citrulline. In an embodiment, the citrulline has an L-configuration.

In some embodiments of bivalent compounds of the disclosure, the active functional groups at the ends (X and X′) are independently selected from carboxyl group, ester group, amide group, and acyl halide group, such as, for example and without limitation, acyl chloride-COCl, or bromo acyl-COBr.

In bivalent compounds of the disclosure, X and X′ may be the same or different. In embodiments where m and/or m′ is 2 or 3, it should be understood that two or more active functional groups at the ends (X, X′) may be the same or different.

In one embodiment of bivalent compounds of the disclosure, A is a methylene, n is 6, n′ is 0, and at least one of m and m′ is not 0.

In some embodiments of bivalent compounds of the disclosure, the bivalent compound has the following structure, or is a pharmaceutically acceptable salt or ester thereof:

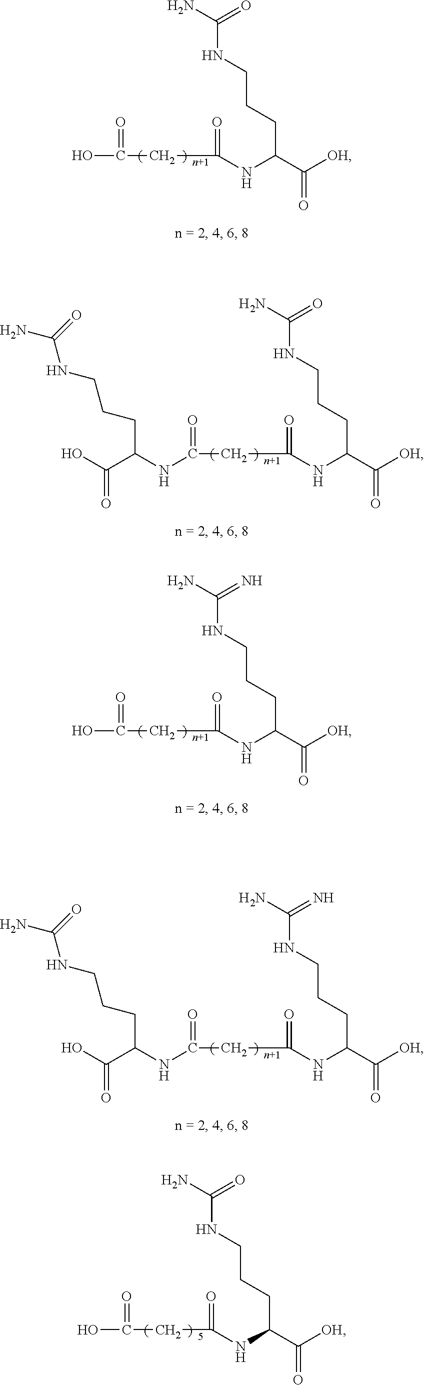

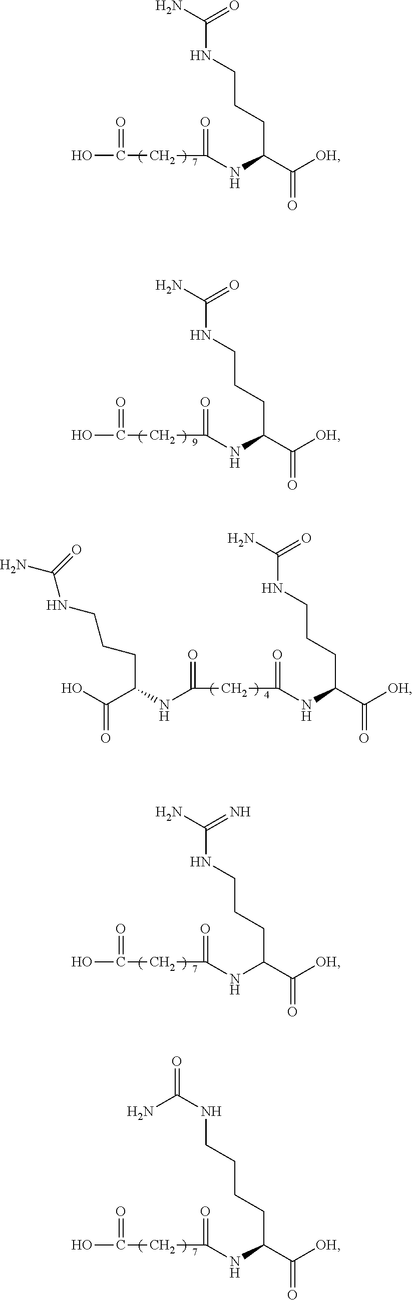

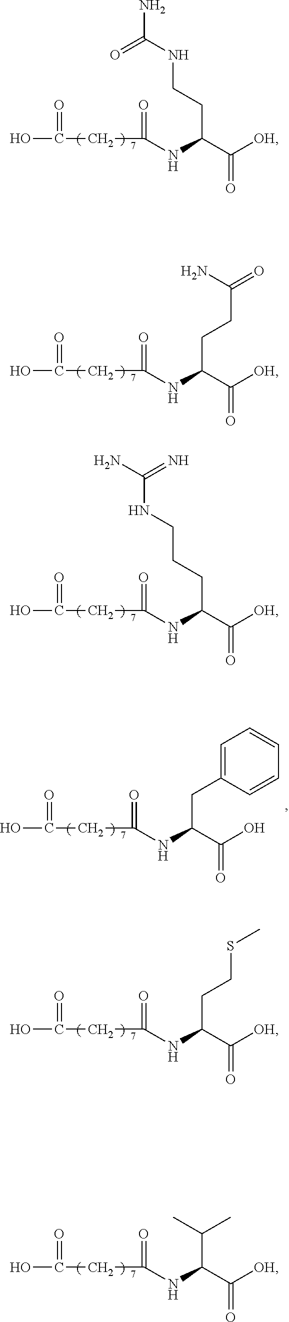

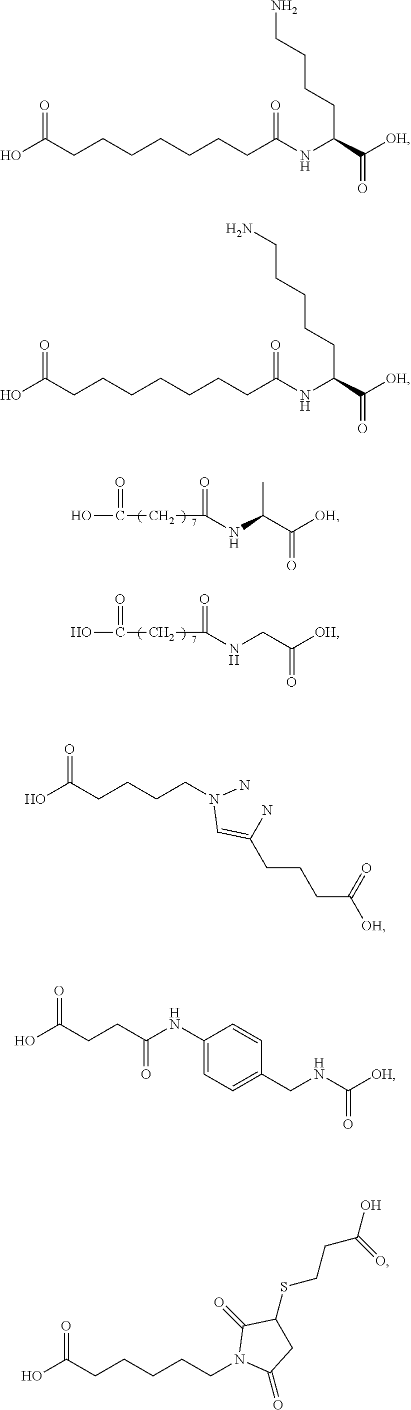

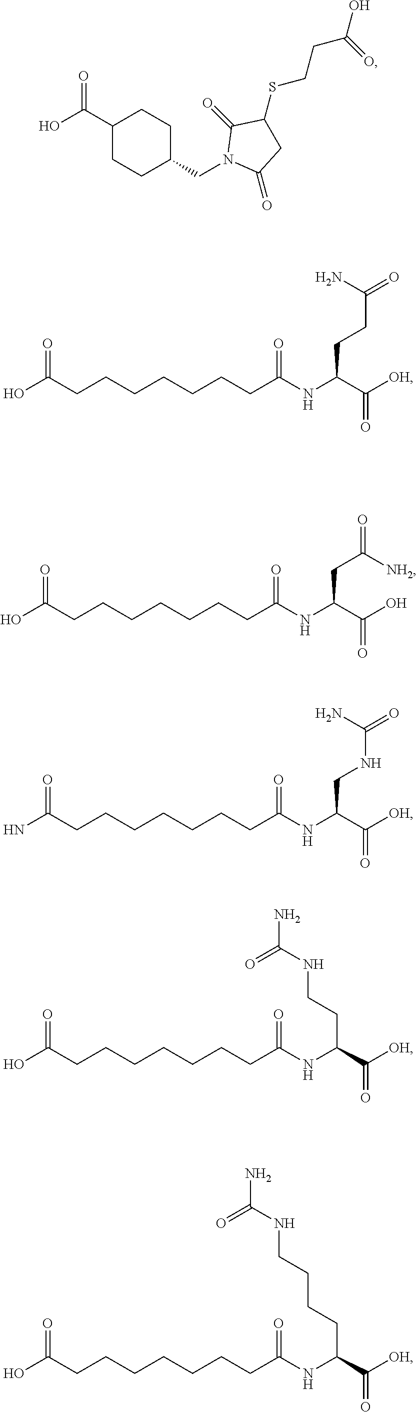

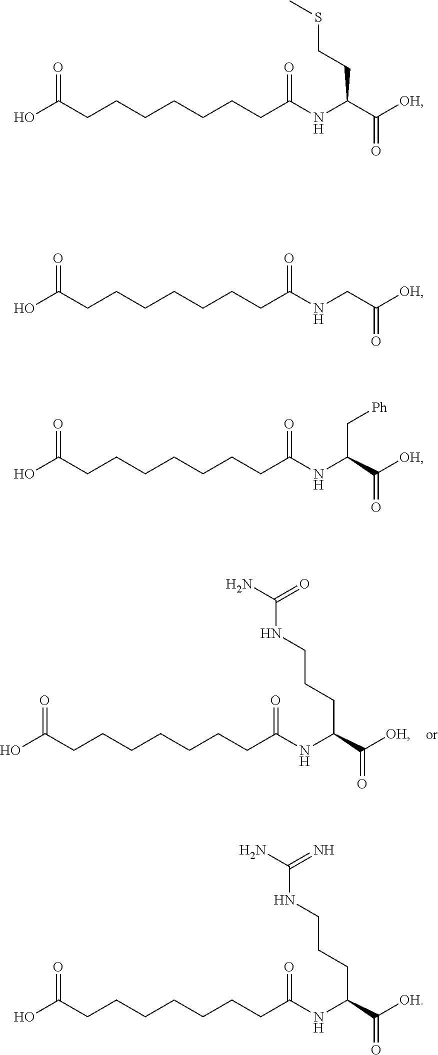 [SMILES: CSCC[C@H](NC(=O)CCCCCCCC(=O)O)C(=O)O ; O=C(O)CCCCCCCC(=O)NCC(=O)O ; O=C(O)CCCCCCCC(=O)N[C@@H](Cc1ccccc1)C(=O)O ; NC(=O)NCCC[C@H](NC(=O)CCCCCCCC(=O)O)C(=O)O ; N=C(N)NCCC[C@H](NC(=O)CCCCCCCC(=O)O)C(=O)O]

In some embodiments of bivalent compounds of the disclosure, the bivalent compound has the following structure, or is a pharmaceutically acceptable salt or ester thereof:

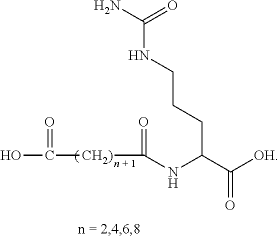

In one embodiment of bivalent compounds of the disclosure, the bivalent compound has the following structure, or is a pharmaceutically acceptable salt or ester thereof:

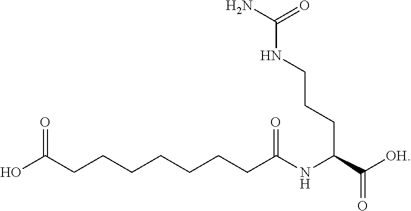 [SMILES: NC(=O)NCCC[C@H](NC(=O)CCCCCCCC(=O)O)C(=O)O]

In some embodiments of bivalent compounds of the disclosure, the bivalent compound is conjugated at one end to one or more targeting moiety and at the other end to one or more bioactive agent, thereby forming a conjugate capable of efficient targeted delivery of the one or more bioactive agent to the cells, tissues or organs targeted by the targeting moiety.

As used herein, the term “targeting moiety” refers to an organic moiety comprising one or more carbohydrate, one or more polypeptide, and/or one or more lipophile component, which is capable of targeting the compound to a desired cell, organ or tissue. For example and without limitation, in some embodiments the targeting moiety binds to a cell-surface receptor on a target cell. The compound may then be internalized into the target cell, e.g. via receptor-mediated endocytosis, and released inside the cell. In this way the targeting moiety can facilitate delivery of the bioactive agent to a target cell, organ, or tissue, where it can act e.g. to treat and/or prevent a disease or disorder in a subject.

In some embodiments, the targeting moiety binds to a cell-surface receptor, such as for example and without limitation, an asialoglycoprotein cell receptor (ASGPR). In one embodiment, the targeting moiety is capable of targeting the compound to hepatic cells and tissues. In an embodiment, the targeting moiety targets the liver. Targeting moieties are described further hereinbelow.

As used herein, the term “bioactive agent” refers to a therapeutic compound or molecule useful for treating or preventing a disease or disorder in a subject. The bioactive agent is not meant to be particularly limited and may be any therapeutic compound or molecule for which targeting to a certain cell or tissue is beneficial. In some embodiments, the bioactive agent is a therapeutic compound for which delivery to the liver is beneficial for treatment or prevention of a disease or disorder in a subject. Non-limiting examples of bioactive agents include antibodies, oligonucleotides, hormones, antibiotics, low molecular weight compounds, drugs, prodrugs, and combinations thereof. In one embodiment, the bioactive agent is an oligonucleotide such as a ribonucleic acid (RNA) or a derivative thereof. For example and without limitation, the bioactive agent may be a functional oligonucleotide, e.g., an RNA therapeutic such as an RNAi agent, e.g., a small interfering RNA (siRNA), an siRNA-drug and/or -prodrug conjugate, an RNA aptamer, or an antisense RNA. Bioactive agents are described further hereinbelow.

In a second broad aspect, there is provided a conjugate or a pharmaceutically acceptable salt or ester thereof, comprising the bivalent compound conjugated to one or more targeting moiety and/or one or more bioactive agent. In some embodiments, the conjugate is a compound of Formula (V):

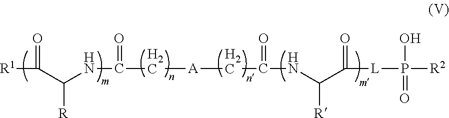

in which the bivalent compound is conjugated to a targeting moiety (R¹) directly and to a bioactive agent (R²) indirectly, through a carrier group —L—P(O)(OH)—, to form the conjugate, wherein A, R, R′, m, m′, n, and n′ are as defined above, and L is a linker.

In some embodiments of Formula (V), R¹ is a targeting moiety, e.g., comprising one or more carbohydrate, polypeptide and/or lipophile; R² is a bioactive agent, e.g., a therapeutic compound or molecule, e.g., comprising an oligonucleotide; and L is a linker moiety, e.g., comprising a nitrogen-containing heterocyclic ring. In some such embodiments, the nitrogen-containing heterocyclic ring is a 4-membered, a 5-membered, or a six-membered ring.

In some embodiments of Formula (V), L is selected from one of the following heterocyclic ring systems:

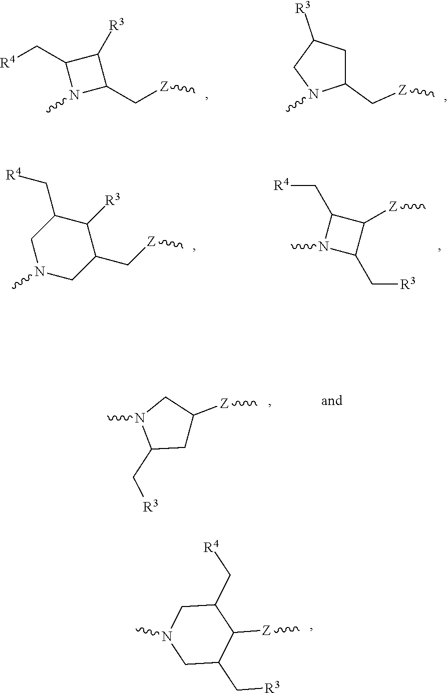

where Z is selected from oxygen (O), sulfur (S), and nitrogen (NH); and R³ and R⁴ are independently H, OH, or OR⁵, wherein R⁵ is a protecting group or a substituent for hydroxyl selected from alky, aryl, alkylaryl, acyl, and phosphonyl group.

In some embodiments of Formula (V), L is connected, via its ring-nitrogen, to one end of the bivalent compound through an amide bond. In some embodiments, alternatively or additionally, L is connected, using Z as a connecting point and through a phosphonyl group, to the bioactive agent.

In one embodiment of Formula (V), L is a five-membered nitrogen-containing heterocyclic ring. In one such embodiment, the 5-membered ring is

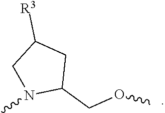

In one embodiment, L comprises a 5-membered ring system having the following steric structure:

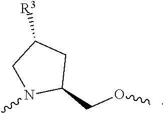

Conjugates of the disclosure are capable of delivering the bioactive agent to a desired target (which is targeted by the targeting moiety). Thus, in some embodiments a conjugate is capable of cell-, tissue-, or organ-specific delivery of the bioactive agent. In some embodiments, a conjugate provides cell-, tissue-, or organ- specific targeting in vivo. In some embodiments, a conjugate is capable of delivering the bioactive agent to the targeted cell, tissue or organ. It should be understood that the targeting moiety is not meant to be particularly limited. Any suitable targeting moiety capable of delivering the bioactive agent to the desired cell, target, or organ may be used. In some embodiments, any targeting moiety capable of binding specifically to a target, e.g., a target cell surface receptor or a target biomarker, is considered to be suitable for use in the conjugates of the disclosure.

In some embodiments, the targeting moiety (R¹) comprises a lipophile. For example and without limitation, the targeting moiety may comprise cholesterol, cholic acid, adamantane acetic acid, 1-pyrenebutyric acid, dihydrotestosterone, 1,3-bis-o (hexadecyl) glycerol, geranyloxyhexyl, hexadecyl glycerol, borneol, menthol, 1,3-propanediol, heptadecyl, palmitic acid, myristic acid, O-3-(oleoyl) lithocholic acid O-3-(oleoyl) cholelenic acid, dimethoxy tribenzyl, phenoxazine, or a combination thereof.

In some embodiments, the targeting moiety (R¹) comprises a carbohydrate. For example and without limitation, the targeting moiety may comprise allose, atrose, arabinose, cladingose, erythritose, erythritulose, fructose, D-fucitol, L-fucitol, fucosamine, fucose, fucoidose, galactosamine, D-galactosamine, N-acetyl-galactosamine (GalNAc), galactose, glucosamine, N-acetyl-glucosamine, glucosaminol, glucose, glucose-6-phosphate, gulose glyceraldehyde, L-glycerol-D-mannose-heptose, glycerol, glycerone, gulose, idulose, lysose, mannosamine, mannose, mannose-6-phosphate, aloulose, quinoose, quinofuramine, rhamnol, rhamnosamine, rhamnose, ribose, ribose Sedum heptanulose, sorbose, tagatose, talose, tartaric acid, threose, xylose, xylulose, or a combination thereof. In one embodiment, the targeting moiety comprises N-acetyl-galactosamine (GalNAc).

In some embodiments, the targeting moiety (R¹) specifically binds to specific receptor(s) of specific tissue(s) to achieve tissue-specific targeting. In some embodiments, the targeting moiety (R¹) specifically target receptors on the surface of hepatocytes, thereby specifically targeting liver tissue. In some embodiments, the targeting moiety (R¹) specifically targets asialoglycoprotein receptors (ASGPRs) on the surface of hepatocytes. In some such embodiments, the targeting moiety (R¹) comprises N-acetyl-galactosamine (GalNAc).

Accordingly, in some embodiments the conjugates deliver the bioactive agent specifically to a tissue expressing the target receptors, e.g., liver tissue. In one such embodiment, the conjugate specifically binds to ASGPRs on the surface of hepatocytes and delivers the bioactive agent to the hepatocytes. In some such embodiments, the conjugate comprises N-acetyl-galactosamine (GalNAc). In some embodiments therefore, the conjugate of the disclosure targets the liver.

In some embodiments, the targeting moiety (R¹) comprises or consists of the following structure:

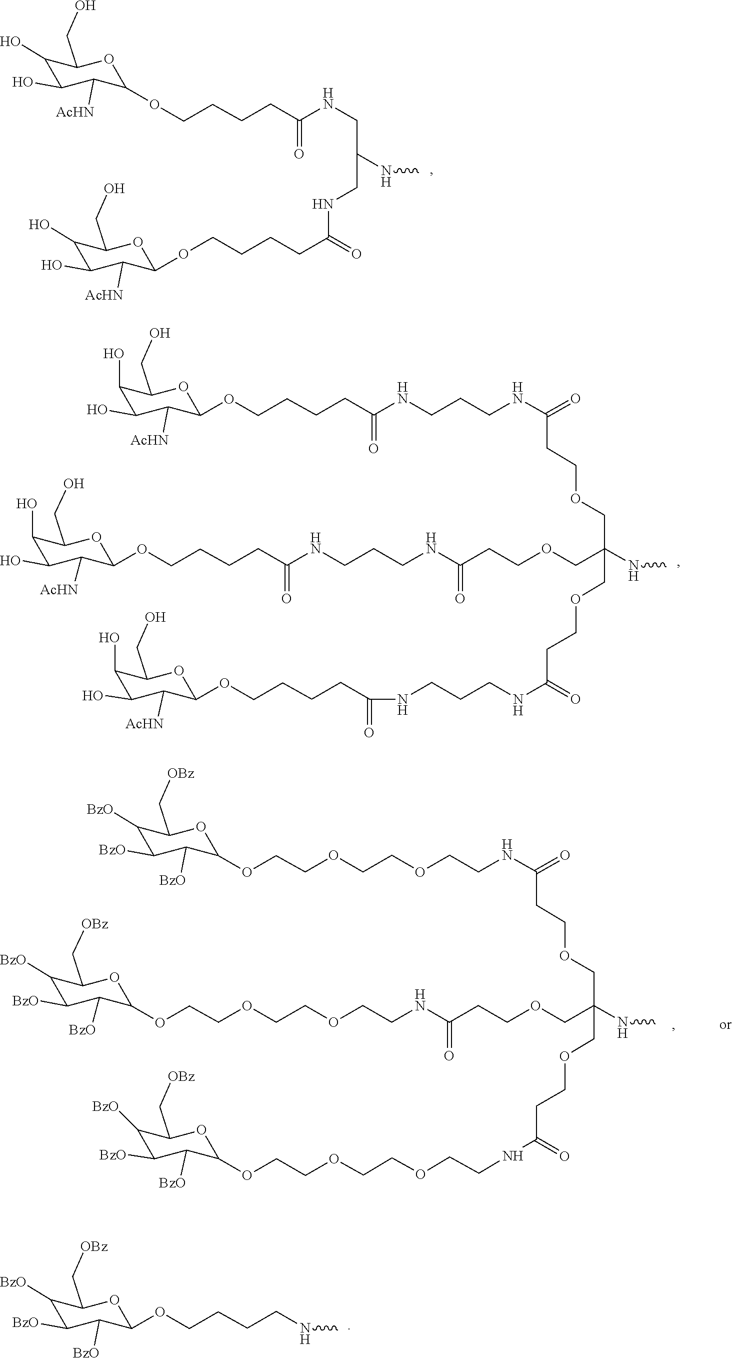

In some embodiments, the conjugates for targeted delivery of bioactive agents in accordance with the disclosure are generated by coupling, in order: a targeting moiety; a bivalent compound as described herein (e.g., a compound of Formula (I)); a linker; a phosphonyl group; and a bioactive agent. In one embodiment, the conjugates for targeted delivery of bioactive agents in accordance with the disclosure are generated by coupling, in order: a targeting moiety; a bivalent compound as described herein (e.g., a compound of Formula (I)); a carrier group comprising a linker and a phosphonyl group, e.g., the carrier group —L—P(O)(OH)—; and a bioactive agent. In some embodiments, the conjugates for targeted delivery of bioactive agents in accordance with the disclosure are generated by coupling, in order: a targeting moiety; a bivalent compound as described herein (e.g., a compound of Formula (I)); and a bioactive agent. In an embodiment, the targeting moiety binds specifically to a cell surface receptor on a target cell. In an embodiment, the targeting moiety binds specifically to liver cells, e.g., hepatocytes, and acts to deliver the conjugate and the bioactive agent specifically and effectively to the liver.

In one embodiment, the conjugate of the disclosure includes, for example and without limitation, a compound set forth in Table 1, or a pharmaceutically acceptable salt or ester thereof, wherein R² is a bioactive agent as defined herein.

In some embodiments, the conjugate of the disclosure comprises or consists of conjugate 1, or a pharmaceutically acceptable salt or ester thereof, wherein R² is a bioactive agent as defined herein.

In some embodiments, the conjugate of the disclosure comprises or consists of conjugate 2, or a pharmaceutically acceptable salt or ester thereof, wherein R² is a bioactive agent as defined herein.

In some embodiments, the conjugate of the disclosure comprises or consists of conjugate 3, or a pharmaceutically acceptable salt or ester thereof, wherein R² is a bioactive agent as defined herein.

In some embodiments, the conjugate of the disclosure comprises or consists of conjugate 4, or a pharmaceutically acceptable salt or ester thereof, wherein R² is a bioactive agent as defined herein.

In some embodiments, the conjugate of the disclosure comprises or consists of conjugate 5, or a pharmaceutically acceptable salt or ester thereof, wherein R² is a bioactive agent as defined herein.

TABLE 1
Examples of Conjugates in accordance with the disclosure.

| | |
|---|---|
| 1 | 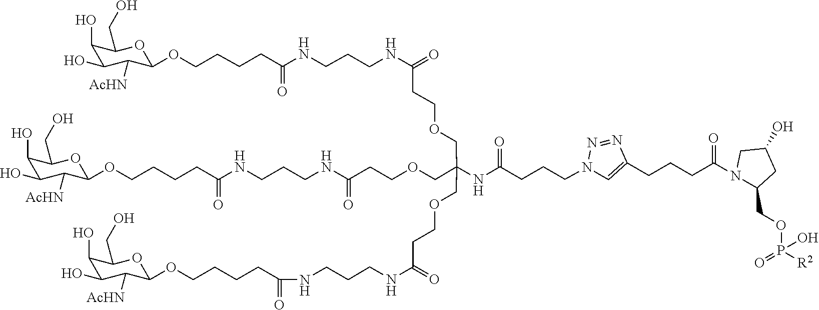 |
| 2 | 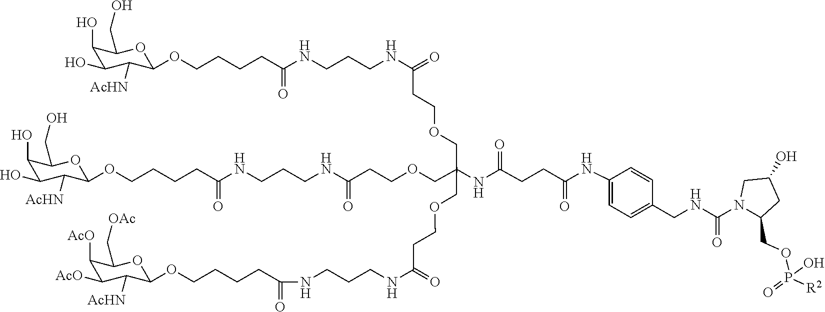 |
| 3 | 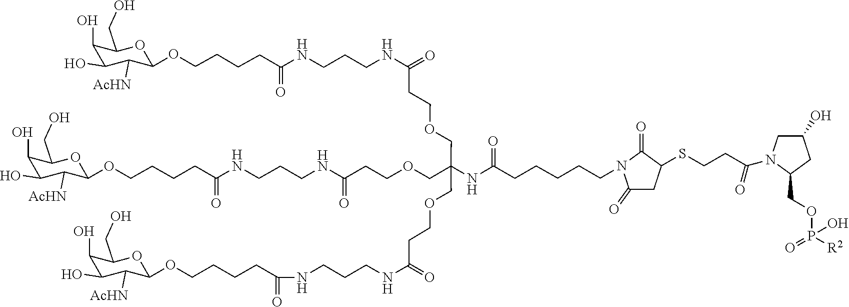 |
| 4 | 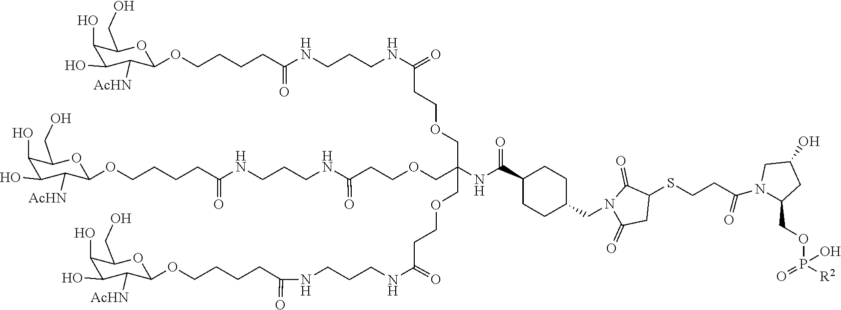 |
| 5 | 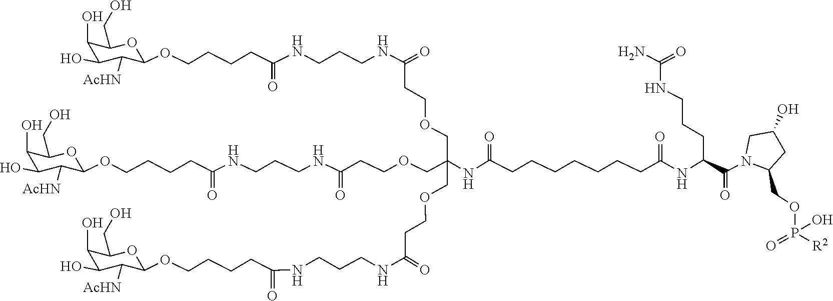 |
| 6 | 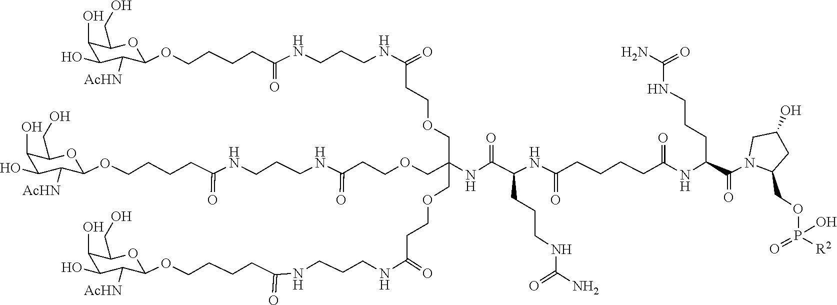 |
| 7 | 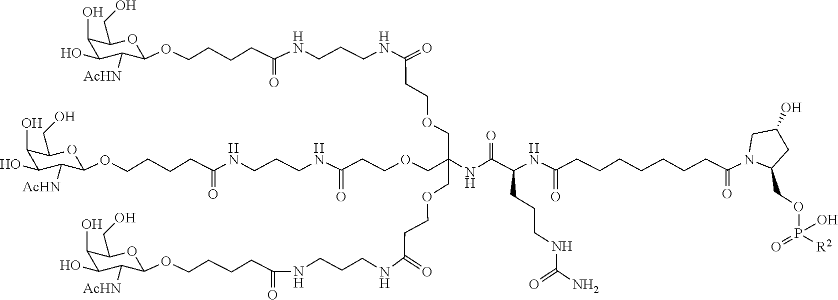 |
| 8 | 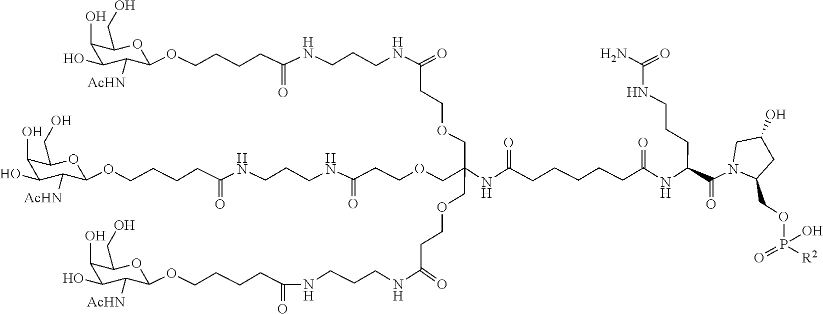 |
| 9 | 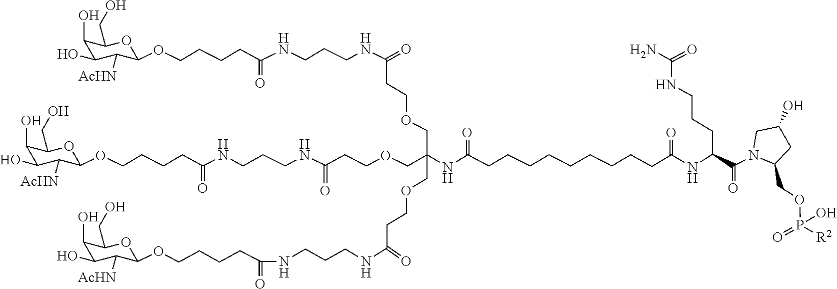 |
| 10 | 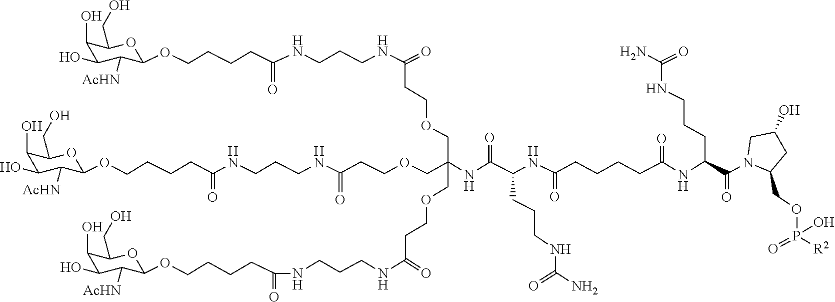 |
| 11 | 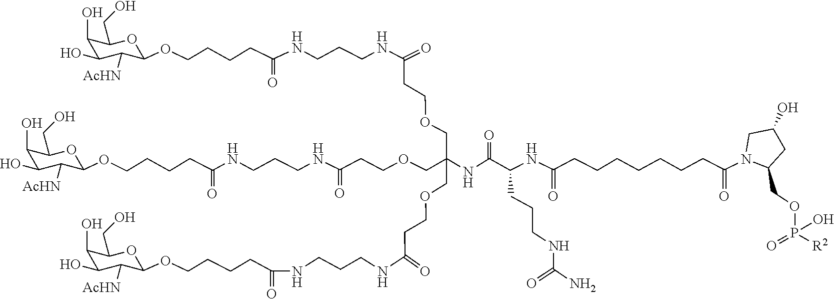 |
| 12 | 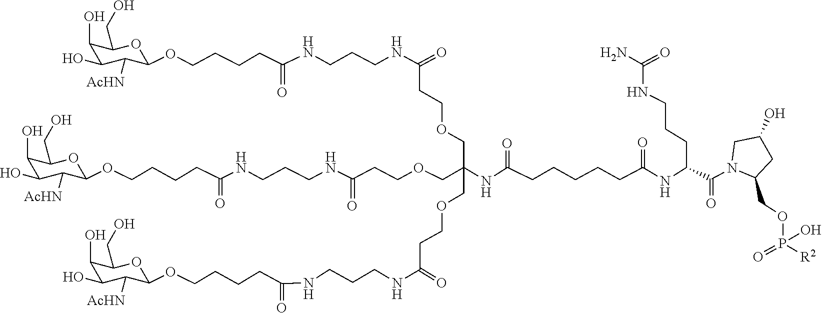 |
| 13 | 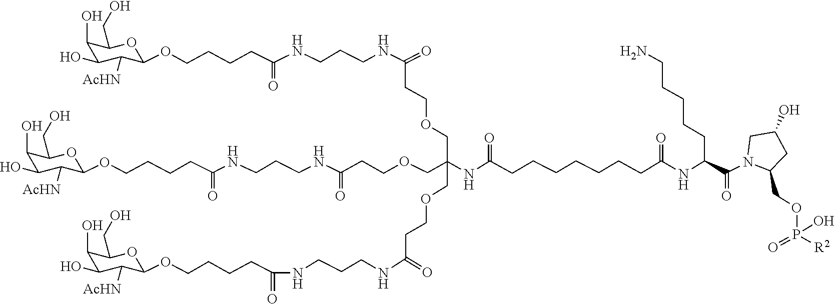 |
| 14 | 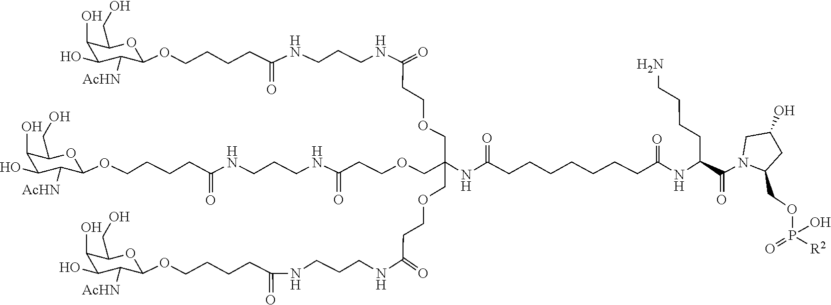 |
| 15 | 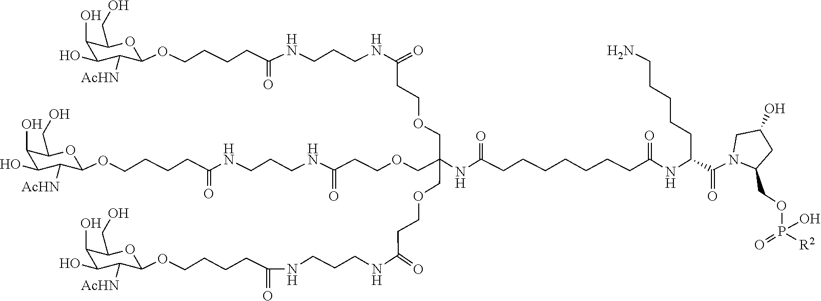 |
| 16 | 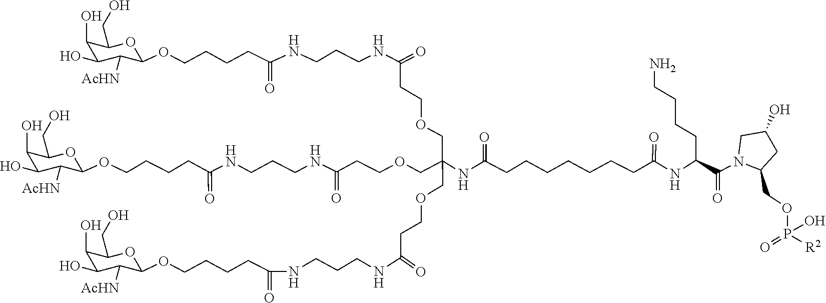 |
| 17 | 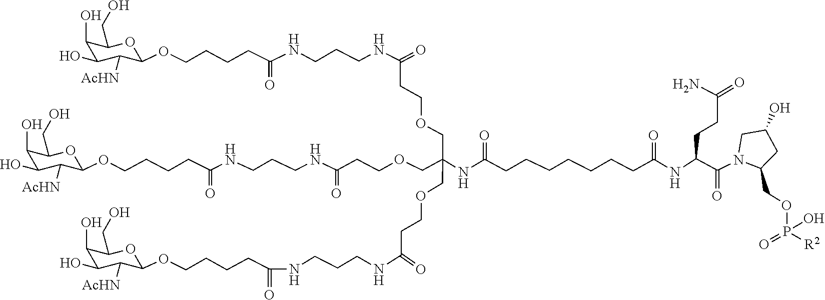 |
| 18 | 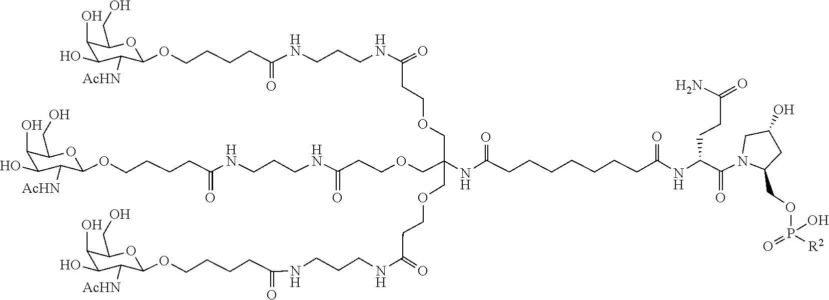 |
| 19 | 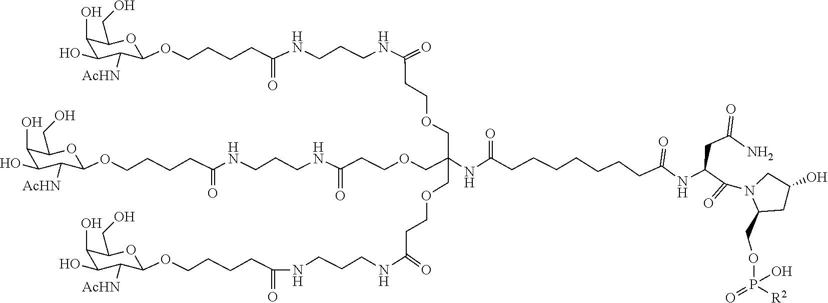 |
| 20 | 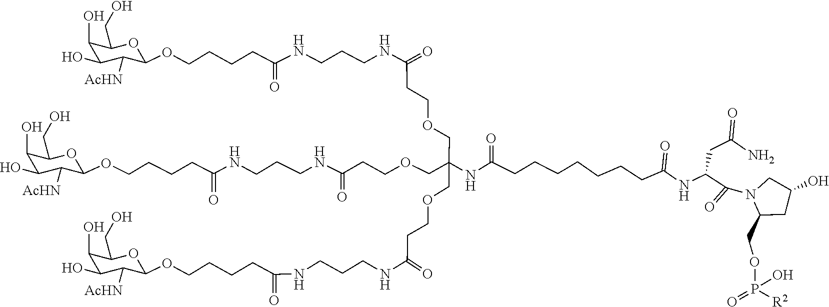 |
| 21 | 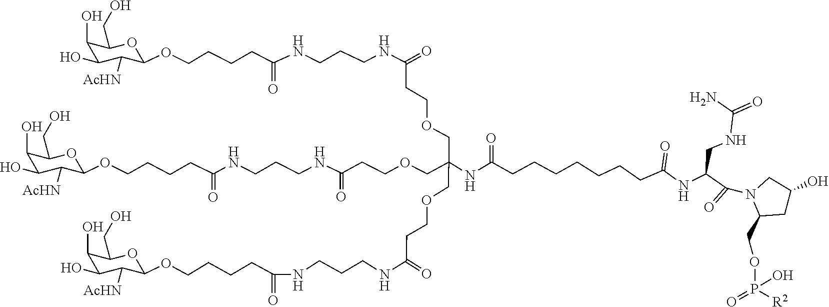 |
| 22 | 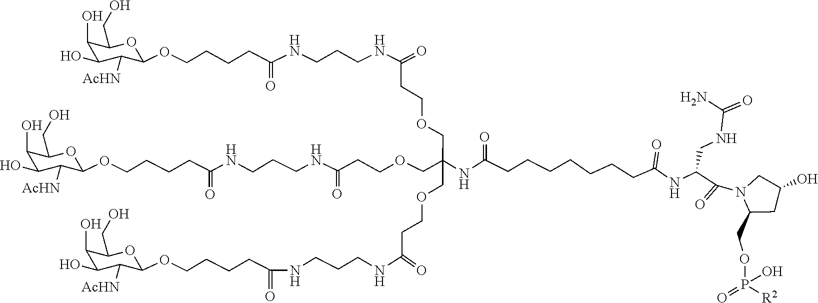 |
| 23 | 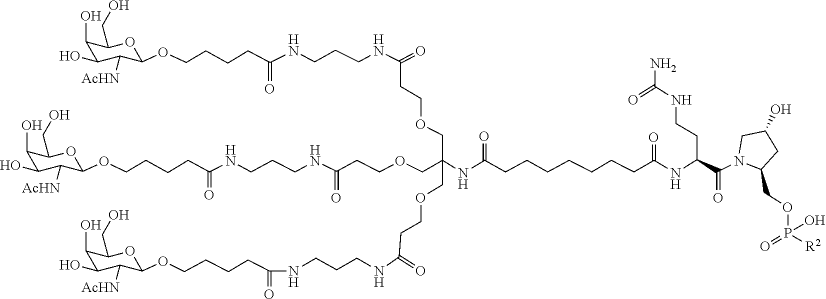 |
| 24 | 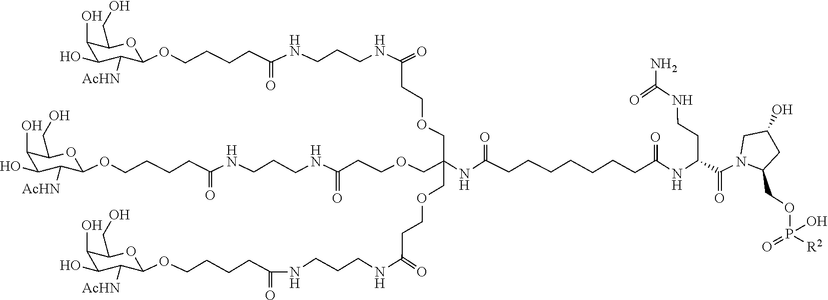 |
| 25 | 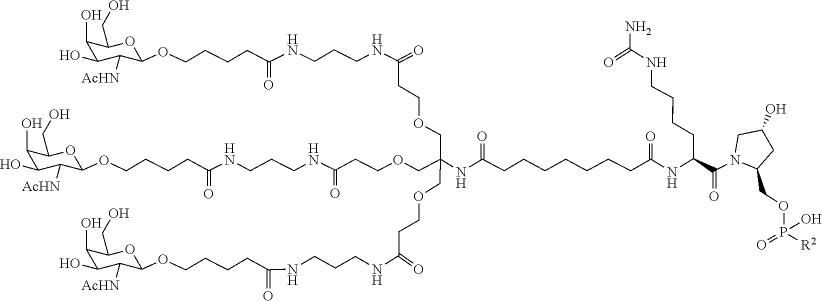 |
| 26 | 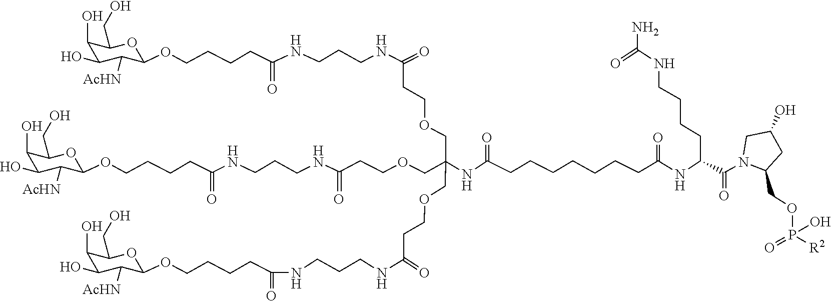 |
| 27 | 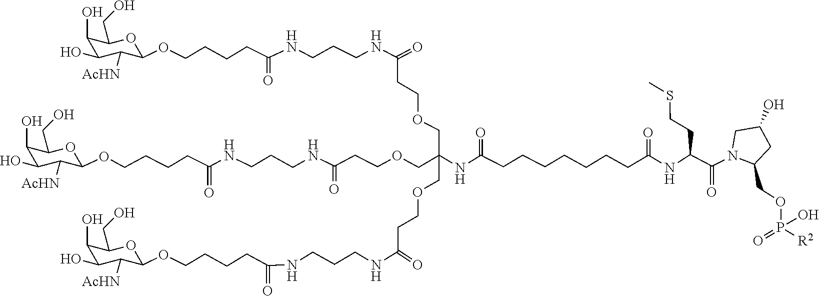 |
| 28 | 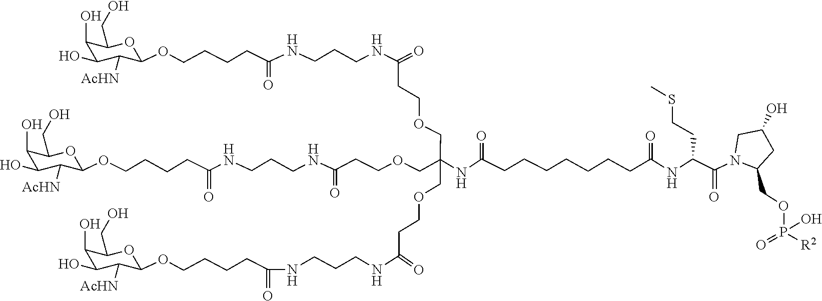 |
| 29 | 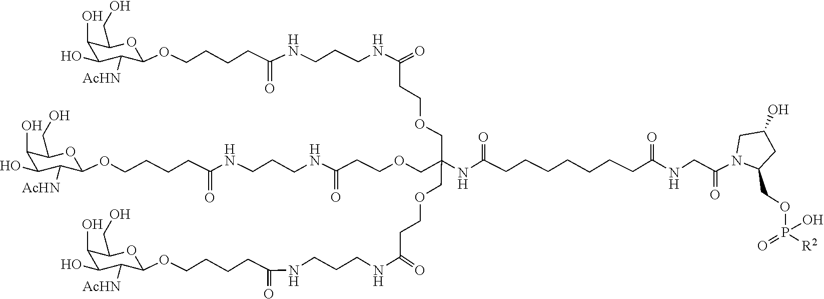 |
| 30 | 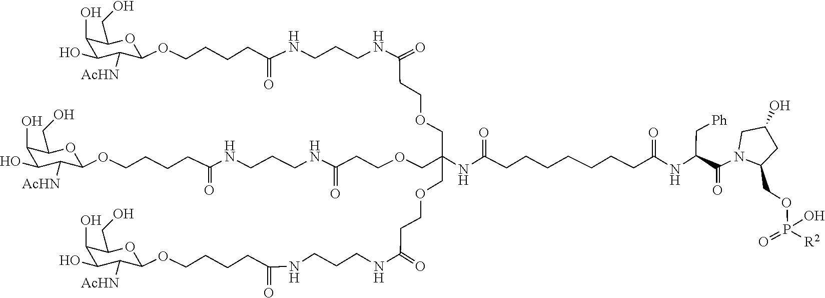 |
| 31 | 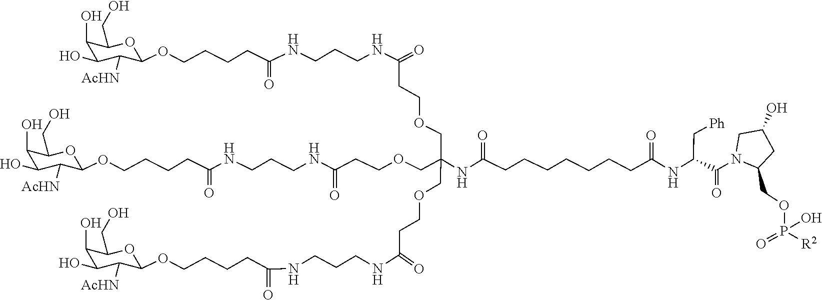 |
| 32 | 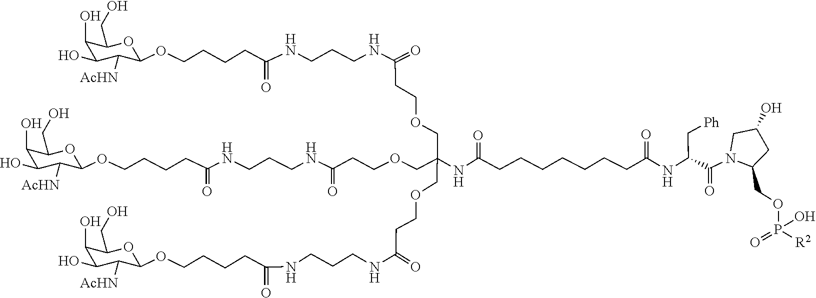 |
| 33 | 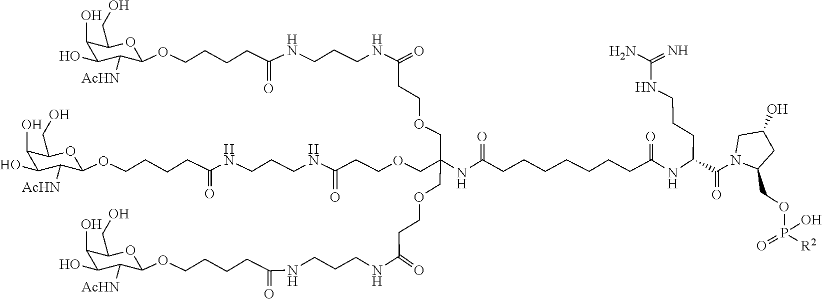 |

In some embodiments, for example and without limitation, R² is a double stranded siRNA comprising or consisting of a sequence listed in Table 2 or Table 3. In some embodiments, the conjugate in Table 1 targets the liver and/or delivers the bioactive agent R² to the liver. In some such embodiments, R² is an siRNA comprising or consisting of the sequence set forth in SEQ ID NO: 1/2. In some such embodiments, R² is an siRNA comprising or consisting of the sequence set forth in SEQ ID NO: 5/6. In some such embodiments, R² is an siRNA comprising or consisting of the sequence set forth in SEQ ID NO: 147/148. In some such embodiments, R² is an siRNA comprising or consisting of the sequence set forth in SEQ ID NO: 179/180.

In one embodiment, the conjugate of the disclosure includes, for example and without limitation, a compound set forth in Table 1, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

In some embodiments, the conjugate of the disclosure comprises or consists of conjugate 1, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

In some embodiments, the conjugate of the disclosure comprises or consists of conjugate 2, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

In some embodiments, the conjugate of the disclosure comprises or consists of conjugate 3, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

In some embodiments, the conjugate of the disclosure comprises or consists of conjugate 4, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

In some embodiments, the conjugate of the disclosure comprises or consists of conjugate 5, or a pharmaceutically acceptable salt or ester thereof, wherein R² is an siRNA comprising or consisting of a sequence listed in Table 2 or Table 3, e.g., the sequence set forth in SEQ ID NO: 1/2, SEQ ID NO: 5/6, SEQ ID NO: 147/148, or SEQ ID NO: 179/180.

It should be understood that compounds of the disclosure (bivalent compounds and/or conjugates) are intended to encompass, but are not limited to, their optical isomers, racemic compounds and other mixtures. In some cases, a single enantiomer or diastereomer, i.e., a single optically active configuration, can be obtained by asymmetric synthesis or by chiral resolution. Resolution of racemates can be achieved, for example, by conventional methods, such as recrystallization in the presence of a resolving agent, or by chiral high-pressure liquid chromatography (HPLC) column chromatography. In addition, some compounds with carbon-carbon double bonds have Z- and e-configurations (or cis and trans configurations). When there is tautomerism in a compound of the disclosure, the term “compound” (including a bivalent compound and/or a conjugate) includes all tautomeric forms of the compound. Such compounds also include crystals and chelates. Similarly, the term “salt” includes all tautomeric forms of a compound and the crystalline form of the compound.

In some embodiments of conjugates of the disclosure, the bioactive agent is a therapeutic compound such as, for example and without limitation, an antibody, an antigen-binding fragment of an antibody, an oligonucleotide (e.g., a functional oligonucleotide such as without limitation an siRNA), a hormone or an antibiotic.

In one embodiment, the bioactive agent is an oligonucleotide. For example and without limitation, the bioactive agent may be an RNAi agent, e.g., an siRNA, an miRNA, an anti-microRNA, a microRNA antagonist, a microRNA mimic, a decoy oligonucleotide, an immunostimulator, a guanine (G) quadruplex DNA or RNA, a guanine (G) tetraplex DNA or RNA, alternative splice, an alternative splice, a single stranded RNA, a double stranded RNA, an antisense nucleic acid (RNA or DNA), an aptamer, a stem loop RNA, an mRNA fragment, or an activating RNA or DNA.

In some embodiments, the bioactive agent is a single stranded oligonucleotide. For example, one end of the bivalent compound is connected to one end of the single stranded oligonucleotide. In some embodiments, the bivalent compound is connected to the first four nucleotides from one end of the single stranded oligonucleotide. In alternate embodiments, the bivalent compound is connected to the end of the single stranded oligonucleotide. In conjugates of the disclosure, the bivalent compound may be connected to the 5′ terminal or the 3′ terminal of an oligonucleotide, e.g., a single stranded oligonucleotide. In one embodiment, the bivalent compound is connected to the 5′ terminal of an oligonucleotide, e.g., a single stranded oligonucleotide, for example in the form of a phosphate ester through the carrier group. In another embodiment, the bivalent compound is connected to the 3′ terminal of an oligonucleotide, e.g., a single stranded oligonucleotide.

In some embodiments, the bioactive agent is a double stranded oligonucleotide. Generally a double stranded oligonucleotide contains both sense and antisense chains. For example, one end of the bivalent compound is connected to one end of the double stranded oligonucleotide. The bivalent compound may be connected to the 5′ terminal or the 3′ terminal of the oligonucleotide, e.g., a double stranded oligonucleotide. In one embodiment, the bivalent compound is connected to the 5′ terminal of the oligonucleotide, e.g., a double stranded oligonucleotide. In another embodiment, the bivalent compound is connected to the 3′ terminal of the oligonucleotide, e.g., a double stranded oligonucleotide.

In some embodiments, an oligonucleotide is an siRNA. In some such embodiments, each nucleotide in the siRNA may be independently modified or unmodified. An siRNA may target any gene for which inhibiting or silencing gene expression has therapeutic benefit. For example and without limitation, an siRNA conjugated to a bivalent compound in accordance with the disclosure may target the ApoB, ApoC, ANGPTL3, PCSK9, SCD1, FVII, p53, HBV, or HCV gene. In some embodiments, an siRNA conjugated to a bivalent compound in accordance with the disclosure is selected from the sequences listed in Table 2 and Table 3. The bivalent compound may be connected, directly or indirectly, to the 3′- or 5′-terminal position of an siRNA.

In one embodiment, an oligonucleotide is an siRNA which targets PCSK9. In one such embodiment, the PCSK9-siRNA has the following sequence:

Sense strand:
(SEQ ID NO: 147)
5′-mC*mUmAmGmAmCCfmUGfmUdTmUmUmGmCmUmUmUmUmGmU-3′,
and
Antisense strand:
(SEQ ID NO: 148)
5′-mA*CPmAAfAfAfmGCfmAAfmAAfmCAfmGGfmUCfmUmAmG*
mA*mA-3′;

where: C, G, U, and A represent cytidine-3′-phosphate, guanosine-3′-phosphate, uridine-3′-phosphate, and adenosine-3′-phosphate, respectively; m means that the adjacent nucleotide on the right side of the letter m is a 2′-O-methyl-modified nucleotide; f means that the adjacent nucleotide on the left side of the letter f is a 2′-fluoro-modified nucleotide; * indicates that the adjacent nucleotide on the left side of * is a thiophosphate-modified nucleotide; indicates that the adjacent nucleotide on the left side of is a nucleotide modified by thiophosphate and 2′-fluorine at the same time; and d means that the adjacent nucleotide to the right of the letter d is a 2′-deoxyribonucleotide.

In another embodiment, an oligonucleotide is an siRNA which targets ANGPTL3. In one such embodiment, the ANGPTL3-siRNA has the following sequence:

Sense strand:
(SEQ ID NO: 179)
5′-mAmAmAmUmCmAmCGfmAAfdAmCmCAfmAmCmUmAmU-3′,
and
Antisense strand:
(SEQ ID NO: 180)
5′-mAUfmAmGmUmUmGGfmUUfmUCfmGUfmGmAmUmUmUCfmC-3′;

where: C, G, U, and A represent cytidine-3′-phosphate, guanosine-3′-phosphate, uridine-3′-phosphate, and adenosine-3′-phosphate respectively; m means that the adjacent nucleotide on the right side of the letter m is a 2′-O-methyl-modified nucleotide; f means that the adjacent nucleotide on the left side of the letter f is a 2′-fluoro-modified nucleotide; * indicates that the adjacent nucleotide on the left side of * is a thiophosphate-modified nucleotide; indicates that the adjacent nucleotide on the left side off * is a nucleotide modified by thiophosphate and 2′-fluorine at the same time; and d means that the adjacent nucleotide to the right of the letter d is a 2′-deoxyribonucleotide.

In another embodiment, a bioactive agent is an oligonucleotide which has the following sequence:

Sense strand:
(SEQ ID NO: 147)
5′-mC*mUmAmGmAmCCfmUGfmUdTmUmUmGmCmUmUmUmUmGmU-3′,
and
Antisense strand:
(SEQ ID NO: 148)
5′-mA*CPmAAfAfAfmGCfmAAfmAAfmCAfmGGfmUCfmUmAmG*mA*
mA-3′;

where: C, G, U, and A represent cytidine-3′-phosphate, guanosine-3′-phosphate, uridine-3′-phosphate, and adenosine-3′-phosphate respectively; m means that the adjacent nucleotide on the right side of the letter m is a 2′-O-methyl-modified nucleotide; f means that the adjacent nucleotide on the left side of the letter f is a 2′-fluoro-modified nucleotide; * indicates that the adjacent nucleotide on the left side of * is a thiophosphate-modified nucleotide; indicates that the adjacent nucleotide on the left side of is a nucleotide modified by thiophosphate and 2′-fluorine at the same time; and d means that the adjacent nucleotide to the right of the letter d is a 2′-deoxyribonucleotide.

In yet another embodiment, a bioactive agent is an oligonucleotide which has the following sequence:

Sense strand:
(SEQ ID NO: 179)
5′-mAmAmAmUmCmAmCGfmAAfdAmCmCAfmAmCmUmAmU-3′,
and
Antisense strand:
(SEQ ID NO: 180)
5′-mAUfmAmGmUmUmGGfmUUfmUCfmGUfmGmAmUmUmUCfmC-3′,

where: C, G, U, and A represent cytidine-3′-phosphate, guanosine-3′-phosphate, uridine-3′-phosphate, and adenosine-3′-phosphate respectively; m means that the adjacent nucleotide on the right side of the letter m is a 2′-O-methyl-modified nucleotide; f means that the adjacent nucleotide on the left side of the letter f is a 2′-fluoro-modified nucleotide; * indicates that the adjacent nucleotide on the left side of * is a thiophosphate-modified nucleotide; indicates that the adjacent nucleotide on the left side of is a nucleotide modified by thiophosphate and 2′-fluorine at the same time; and d means that the adjacent nucleotide to the right of the letter d is a 2′-deoxyribonucleotide.

In yet another embodiment, a bioactive agent is an siRNA having a sequence set forth in Table 2. Non-limiting exemplary sequences of siRNAs for use as bioactive agents and in conjugates of the disclosure are given in Table 2.

Nucleic acid sequences of the disclosure, including sequences in Tables 2 and 3, are represented as follows:
“C”, “G”, “U”, and “A” represent cytidine-3′-phosphate, guanosine-3′-phosphate, uridine-3′-phosphate, and adenosine-3′-phosphate, respectively.
“T” represents 5′-methyluridine-3′-phosphate.
“m” means that the adjacent nucleotide on the right side of the letter m is a 2′-O-methyl-modified (2′-OMe) nucleotide. For example, “mA” represents 2′-O-methyladenosine-3′-phosphate.
“f” means that the adjacent nucleotide on the left side of the letter f is a 2′-fluoro-modified nucleotide. For example, “Af” represents 2′-fluoroadenosine-3′-phosphate.
“*” indicates that the adjacent nucleotide on the left side of * is a thiophosphate-modified nucleotide. For example, “A*” represents adenosine-3′-phosphorothioate.
“P” indicates that the adjacent nucleotide on the left side of is a nucleotide modified by thiophosphate and 2′-fluorine at the same time. For example, “Af*” represents 2′-fluoroadenosine-3′-phosphorothioate.
“d” means that the adjacent nucleotide to the right of the letter d is a 2′-deoxyribonucleotide. For example, “dT” represents 2′-deoxythymidine.
“mX*” means that the nucleotide between “m” and “*” is a nucleotide which is both 2′-O-methyl-modified and thiophosphate-modified. For example, “mA*” represents 2′-O-methyladenosine-3′-phosphorothioate.
“o” means that the adjacent nucleotide to the right of the letter o is a 2 ‘-methoxyethyl modified nucleotide. For example, “oG” represents 2’-O-methoxyethylguanosine-3′-phosphate.
“P” represents 5′-phosphate.
“S” means sense.
“AS” means antisense.

TABLE 2
Examples of siRNAs in accordance with the disclosure.

| | | | SEQ ID |
|---|---|---|---|
| TARGET | SEQUENCE (5′→3′) | | NO. |
| PCSK9 | S | mC*mU*mAmGmAmCCfmUGfmUdTmUmUmGmCmUm | 1 |
| | | UmUmUmGmU | |
| | AS | mA*Cf*mAAfAfAfmGCfmAAfmAAfmCAfmGGfmUCfm | 2 |
| | | UmAmG*mA*mA | |
| PCSK9 | S | GmCmCmUGGAGmUmUmUAmUmUmCGGAAdTdT | 3 |
| | AS | PUUCCGAAUAAACUCCAGGCdT*dT | 4 |
| PCSK9 | S | GmCmCmUGGAGmUmUmUAmUmUmCGGAAdTdT | 5 |
| | AS | PmUUfmCCfmGAfmAUfmAAfmACfmUCfmCAfmGGfm | 6 |
| | | CdTdT | |
| PCSK9 | S | GmCmCmUGGAGmUmUmUAmUmUmCGGAdTdT | 7 |
| | AS | PUUCCGAAUAAACUCCAGGCdT*dT | 8 |
| PCSK9 | S | GmCmCmUGGAGmUmUmUAmUmUmCGGAAdTdT | 9 |
| | AS | PUUCCGAAUAAACUCCAGGCdT*dT | 10 |
| PCSK9 | S | GmCmCmUGGAGmUmUmUAmUmUmCGGAAdTdT | 11 |
| | AS | PUUCCGAAUAAACUCCAGGCdT*dT | 12 |
| PCSK9 | S | GmCmCmUGGAGmUmUmUAmUmUmCGGAAdTdT | 13 |
| | AS | PmUUfmCCfmGAfmAUfmAAfmACfmUCfmCAfmGGfm | 14 |
| | | CdTdT | |
| PCSK9 | S | GmCmCmUGGAGmUmUmUAmUmUmCGGAAdT*dT | 15 |
| | AS | PUUCCGAAUAAACUCCAGGCdT*dT | 16 |
| PCSK9 | S | GmCmCmUGGAGmUmUmUAmUmUmCGGAAdT*dT | 17 |
| | AS | GmCmCmUGGAGmUmUmUAmUmUmCGGAAdTdT | 18 |
| ApoB | S | GGAAUCmUmUAmUAmUmUmUGAUCmCA*A | 19 |
| | AS | mUmUGGAUmCAAAmUAmUAAGAmUUCmC*mC*U | 20 |
| ApoB | S | GGAAUCmUmUAmUAmUmUmUGAUCmCAA | 21 |
| | AS | UmUGGAUmCAAAmUAmUAAGAmUUCmC*mC*U | 22 |
| ApoB | S | GGAAUCmUmUAmUAmUmUmUGAUCmCAA | 23 |
| | AS | mUmUGGAUmCAAAmUAmUAAGAmUUCmC*mC*U | 24 |
| ApoB | S | GGAAUCmUmUAmUAmUmUmUGAUCmCAA | 25 |
| | AS | mUmUGGAUmCAAAmUAmUAAGAmUUCmC*mC*U | 26 |
| ApoB | S | GGAAUCmUmUAmUAmUmUmUGAUCmCAA | 27 |
| | AS | mUmUGGAUmCAAAmUAmUAAGAmUUCmCmC*U | 28 |
| ApoB | S | GGAAUCmUmUAmUAmUmUmUGAUCmCAA | 29 |
| | AS | mUmUGGAUmCAAAmUAmUAAGAmUUCmC*mC*U | 30 |
| ApoB | S | GGAAUCmUmUAmUAmUmUmUGAUCmCAA | 31 |
| | AS | mUmUGGAUmCAAAmUAmUAAGAmUUCmC*mC*U | 32 |
| ApoB | S | GGAAUCmUmUAmUAmUmUmUGAUCmCAA | 33 |
| | AS | mUmUGGAUmCAAAmUAmUAAGAmUUCmC*mC*U | 34 |
| ApoB | S | GGAAUCmUmUAmUAmUmUmUGAUCmCAA | 35 |
| | AS | mUmUGGAUmCAAAmUAmUAAGAmUUCmC*mC*U | 36 |
| ApoB | S | GGAAUCmUmUAmUAmUmUmUGAUCmCAA | 37 |
| | AS | mUmUGGAUmCAAAmUAmUAAGAmUUCmC*mC*U | 38 |
| ApoB | S | GGAAUCmUmUAmUAmUmUmUGAUCmCAA | 39 |
| | AS | mUmUGGAUmCAAAmUAmUAAGAmUUCmC*mC*U | 40 |
| ApoB | S | GGAAUCmUmUAmUAmUmUmUGAUCmCAA | 41 |
| | AS | mUmUGGAUmCAAAmUAmUAAGAmUUCmC*mC*U | 42 |
| ApoB | S | GGAAUCmUmUAmUAmUmUmUGAUCmCAA | 43 |
| | AS | mUmUGGAUmCAAAmUAmUAAGAmUUCmC*mC*U | 44 |
| ApoB | S | GGAAUCmUmUAmUAmUmUmUGAUCmCAA | 45 |
| | AS | mUmUGGAUmCAAAmUAmUAAGAmUUCmC*mC*U | 46 |
| ApoB | S | GGAAUCmUmUAmUAmUmUmUGAUCmCAA | 47 |
| | AS | mUmUGGAUmCAAAmUAmUAAGAmUUCmC*mC*U | 48 |
| ApoB | S | GGAAUCmUmUAmUAmUmUmUGAUCmCAA | 49 |
| | AS | mUmUGGAUmCAAAmUAmUAAGAmUUCmC*mC*U | 50 |
| ApoB | S | GGAAUCmUmUAmUAmUmUmUGAUCmCAA | 51 |
| | AS | mUmUGGAUmCAAAmUAmUAAGAmUUCmC*mC*U | 52 |
| ApoB | S | GGAAUCmUmUAmUAmUmUmUGAUCmCAA | 53 |
| | AS | mUmUGGAUmCAAAmUAmUAAGAmUUCmC*mC*U | 54 |
| ApoB | S | GGAAUCmUmUAmUAmUmUmUGAUCmCAA | 55 |
| | AS | mUmUGGAUmCAAAmUAmUAAGAmUUCmC*mC*U | 56 |
| ApoB | S | GGAAUCmUmUAmUAmUmUmUGAUCmCAA | 57 |
| | AS | mUmUGGAUmCAAAmUAmUAAGAmUUCmC*mC*U | 58 |
| ApoB | S | GGAAUCmUmUAmUAmUmUmUGAUCmCAA | 59 |
| | AS | mUmUGGAUmCAAAmUAmUAAGAmUUCmC*mC*U | 60 |
| ApoB | S | GGAAUCmUmUAmUAmUmUmUGAUCmCAA | 61 |
| | AS | mUmUGGAUmCAAAmUAmUAAGAmUUCmC*mC*U | 62 |
| ApoB | S | GGAAUCmUmUAmUAmUmUmUGAUCmCAA | 63 |
| | AS | mUmUGGAUmCAAAmUAmUAAGAmUUCmC*mC*U | 64 |
| FVII | S | GGAUfCfAUfCfUfCfAAGUfCfUfUfACfdTdT | 65 |
| | AS | GUfAAGACfUfUfGAGAUfGAUfCfCfdT*dT | 66 |
| FVII | S | GGAUfCfAUfCfUfCfAAGUfCfUfUfACfdTdT | 67 |
| | AS | GUfAAGACfUfUfGAGAUfGAUfCfCfdT*dT | 68 |
| FVII | S | GGAUfCfAUfCfUfCfAAGUfCfUfUfACfdTdT | 69 |
| | AS | GUfAAGACfUfUfGAGAUfGAUfCfCfdT*dT | 70 |
| FVII | S | GGAUfCfAUfCfUfCfAAGUfCfUfUfACfdTdT | 71 |
| | AS | GUfAAGACfUfUfGAGAUfGAUfCfCfdT*dT | 72 |
| FVII | S | GGAUfCfAUfCfUfCfAAGUfCfUfUfACfdTdT | 73 |
| | AS | GUfAAGACfUfUfGAGAUfGAUfCfCfdT*dT | 74 |
| FVII | S | GGAUfCfAUfCfUfCfAAGUfCfUfUfACfdTdT | 75 |
| | AS | GUfAAGACfUfUfGAGAUfGAUfCfCfdT*dT | 76 |
| FVII | S | GGAUCAUCUCAAGUCUUACdT*dT | 77 |
| | AS | GUfAAGACfUfUfGAGAUfGAUfCfCfdT*dT | 78 |
| FVII | S | GGAUfCfAUfCfUfCfAAGUfCfUfUfACfdTdT | 79 |
| | AS | GUfAAGACfUfUfGAGAUfGAUfCfCfdT*dT | 80 |
| FVII | S | GGAUfCfAUfCfUfCfAAGUfCfUfUfACfdTdT | 81 |
| | AS | GUfAAGACfUfUfGAGAUfGAUfCfCfdT*dT | 82 |
| FVII | S | GGAUCAUCUCAAGUCUUACdTdT | 83 |
| | AS | GUAAGACUUGAGAUGAUCCdTdT | 84 |
| FVII | S | GGAUfCfAUfCfUfCfAAGUfCfUfUfACfdTdT | 85 |
| | AS | GUfAAGACfUfUfGAGAUfGAUfCfCfdT*dT | 86 |
| FVII | S | GGAUfCfAUfCfUfCfAAGUfCfUfUfACfdTdT | 87 |
| | AS | GUfAAGACfUfUfGAGAUfGAUfCfCfdT*dT | 88 |
| FVII | S | GGAUfCfAUfCfUfCfAAGUfCfUfUfACfdTdT | 89 |
| | AS | GUfAAGACfUfUfGAGAUfGAUfCfCfdT*dT | 90 |
| FVII | S | GGAUfCfAUfCfUfCfAAGUfCfUfUfACfdTdT | 91 |
| | AS | GUfAAGACfUfUfGAGAUfGAUfCfCfdT*dT | 92 |
| FVII | S | GGAUfCfAUfCfUfCfAAGUfCfUfUfACfdTdT | 93 |
| | AS | GUfAAGACfUfUfGAGAUfGAUfCfCfdT*dT | 94 |
| FVII | S | GGAUfCfAUfCfUfCfAAGUfCfUfUfACfdTdT | 95 |
| | AS | GUfAAGACfUfUfGAGAUfGAUfCfCfdT*dT | 96 |
| FVII | S | GGAUfCfAUfCfUfCfAAGUfCfUfUfACfdTdT | 97 |
| | AS | GUfAAGACfUfUfGAGAUfGAUfCfCfdT*dT | 98 |

In one embodiment of conjugates of the disclosure, the bioactive agent is an siRNA having the sequence set forth in SEQ ID NO: 1/2. This sequence is substantially the same as the sequence of Inclisiran, which is used as positive control herein. The specific sequence is the following:

Sense:
(SEQ ID NO: 1)
5′-mC*mU*mAmGmAmCCfmUGfmUdTmUmUmGmCmUmUmUmUmGmU;
and
Antisense:
(SEQ ID NO: 2)
5′-mA*CPmAAfAfAfmGCfmAAfmAAfmCAfmGGfmUCfmUmAmG*
mA*mA.

In one embodiment of conjugates of the disclosure, the bioactive agent is an siRNA comprising or consisting of the sequence set forth in SEQ ID NO: 1/2, i.e., comprising or consisting of the following:

Sense:
(SEQ ID NO: 1)
5′-mC*mU*mAmGmAmCCfmUGfmUdTmUmUmGmCmUmUmUmUmGmU;
and
Antisense:
(SEQ ID NO: 2)
5′-mA*CPmAAfAfAfmGCfmAAfmAAfmCAfmGGfmUCfmUmAmG*mA*mA.

In one embodiment of conjugates of the disclosure, the bioactive agent is an siRNA having the sequence set forth in SEQ ID NO: 5/6. This sequence is substantially the same as the sequence of Inclisiran, which is used as positive control herein. The specific sequence is the following:

Sense:
(SEQ ID NO: 5)
5′-GmCmCmUGGAGmUmUmUAmUmUmCGGAAdTdT-3′;
and
Antisense:
(SEQ ID NO: 6)
5′-PmUUfmCCfmGAfmAUfmAAfmACfmUCfmCAfmGGfmCdTdT-3′.

In one embodiment of conjugates of the disclosure, the bioactive agent is an siRNA comprising or consisting of the sequence set forth in SEQ ID NO: 5/6, i.e., comprising or consisting of the following:

Sense:
(SEQ ID NO: 5)
5′-GmCmCmUGGAGmUmUmUAmUmUmCGGAAdTdT-3′;
and
Antisense:
(SEQ ID NO: 6)
5′-PmUUfmCCfmGAfmAUfmAAfmACfmUCfmCAfmGGfmCdTdT-3′.

In yet another embodiment, a bioactive agent is an siRNA having a sequence set forth in Table 3. Non-limiting exemplary sequences of siRNAs for use as bioactive agents and in conjugates of the disclosure are given in Table 3.

TABLE 3
Examples of siRNAs in
accordance with the disclosure.

| | | | SEQ |
|---|---|---|---|
| | | | ID |
| TARGET | SEQUENCE (5′→3′) | | NO. |
| PCSK9 | S | mCmUGfmUGfmCmUAfGfmCdAAfmCAfmCmCmCmAmA | 99 |
| | AS | mU*Uf*mGGfmGUfmGmUmUmGCfUfmAGfmCAfmCAf | 100 |
| | | mG*Cf*mC | |
| PCSK9 | S | mCmUGfmUGfmCmUAfGfmCdAAfmCmAmCmCmCmAmA | 101 |
| | AS | mU*Uf*mGGfmGUfmGmUmUmGCfUfmAGfmCAfmCAf | 102 |
| | | mG*Cf*mC | |
| PCSK9 | S | mC*mU*GfmUGfmCmUAfGfmCdAAfmCmAmCmCmCmA | 103 |
| | | mA | |
| | AS | mU*UPmGGfmGUfmGmUmUmGCfUfmAGfmCAfmCAfm | 104 |
| | | G*Cf*mC | |
| PCSK9 | S | mC*mUGfmUGfmCmUAfGfmCdAAfmCmAmCmCmCmAm | 105 |
| | | A | |
| | AS | mU*UPmGGfmGUfmGmUmUmGCfUfmAGfmCAfmCAfm | 106 |
| | | G*Cf*mC | |
| PCSK9 | S | mCmU*GfmUGfmCmUAfGfmCdAAfmCmAmCmCmCmAm | 107 |
| | | A | |
| | AS | mU*UPmGGfmGUfmGmUmUmGCfUfmAGfmCAfmCAfm | 108 |
| | | G*Cf*mC | |
| PCSK9 | S | mCmUGfmUGfmCmUAfGfmCdAAfmCmAmCmCmCmAmA | 109 |
| | AS | mUUfmGGfmGUfmGmUmUmGCfUfmAGfmCAfmCAfmG | 110 |
| | | CfmC | |
| PCSK9 | S | mCmUGfmUGfmCmUAfGfmCdAAfmCmAmCmCmCmAmA | 111 |
| | AS | mUdTmGGfmGUfmGmUmUmGCfUfmAGfmCAfmCAfmG | 112 |
| | | CfmC | |
| PCSK9 | S | mCmUGfmUGfmCmUAfGfmCdAAfmCmAmCmCmCmAmA | 113 |
| | AS | mUUfmGGfmGUfmGmUmUmGCfUfmAoGmCAfmCAfmG | 114 |
| | | CfmC | |
| PCSK9 | S | mCmUGfmUGfmCmUAfGfmCdAAfmCmAmCmCmCmAmA | 115 |
| | AS | mU*Uf*mGGfmGUfmGmUmUmGCfUfmAGfmCAfmCAf | 116 |
| | | mG*CUmC | |
| PCSK9 | S | mCmUGfmUGfmCmUAfGfmCdAAfmCmAmCmCmCmAmA | 117 |
| | AS | mU*UfmGGfmGUfmGmUmUmGCfUfmAGfmCAfmCAfm | 118 |
| | | G*Cf*mC | |
| PCSK9 | S | mCmUGfmUGfmCmUAfGfmCdAAfmCmAmCmCmCmAmA | 119 |
| | AS | mU*UfmGGfmGUfmGmUmUmGCfUfmAGfmCAfmCAfm | 120 |
| | | GCf*mC | |
| PCSK9 | S | CUGUGCUAGCdAACACCCAA | 121 |
| | AS | UfUfGfmGGfmUGfmUmUmGCfmUAfGfCfmACfmAGf | 122 |
| | | mCCf | |
| PCSK9 | S | CUGUGCUAGCdAACACCCAA | 123 |
| | AS | mUUfGfmGGfmUGfmUmUmGCfmUAfGfCfmACfmAGf | 124 |
| | | mCCf | |
| PCSK9 | S | CUGUGCUAGCdAACACCCAA | 125 |
| | AS | UfmUGfmGGfmUGfmUmUmGCfmUAfGfCfmACfmAGf | 126 |
| | | mCCf | |
| PCSK9 | S | CUGUGCUAGCdAACACCCAA | 127 |
| | AS | UfUfmGmGGfmUGfmUmUmGCfmUAfGfCfmACfmAGf | 128 |
| | | mCCf | |
| PCSK9 | S | CUGUGCUAGCdAACACCCAA | 129 |
| | AS | UfUfGfmGmGmUGfmUmUmGCfmUAfGfCfmACfmAGf | 130 |
| | | mCCf | |
| PCSK9 | S | CUGUGCUAGCdAACACCCAA | 131 |
| | AS | UfUfGfmGGfmUmGmUmUmGCfmUAfGfCfmACfmAGf | 132 |
| | | mCCf | |
| PCSK9 | S | CUGUGCUAGCdAACACCCAA | 133 |
| | AS | UfUfGfmGGfmUGfmUmUmGmCmUAfGfCfmACfmAGf | 134 |
| | | mCCf | |
| PCSK9 | S | CUGUGCUAGCdAACACCCAA | 135 |
| | AS | UfUfGfmGGfmUGfmUmUmGCfmUmAGfCfmACfmAGf | 136 |
| | | mCCf | |
| PCSK9 | S | CUGUGCUAGCdAACACCCAA | 137 |
| | AS | UfUfGfmGGfmUGfmUmUmGCfmUAfmGCfmACfmAGf | 138 |
| | | mCCf | |
| PCSK9 | S | CUGUGCUAGCdAACACCCAA | 139 |
| | AS | UfUfGfmGGfmUGfmUmUmGCfmUAfGfmCmACfmAGf | 140 |
| | | mCCf | |
| PCSK9 | S | CUGUGCUAGCdAACACCCAA | 141 |
| | AS | UfUfGfmGGfmUGfmUmUmGCfmUAfGfCfmAmCmAGf | 142 |
| | | mCCf | |
| PCSK9 | S | CUGUGCUAGCdAACACCCAA | 143 |
| | AS | UfUfGfmGGfmUGfmUmUmGCfmUAfGfCfmACfmAmG | 144 |
| | | mCCf | |
| PCSK9 | S | CUGUGCUAGCdAACACCCAA | 145 |
| | AS | UfUfGfmGGfmUGfmUmUmGCfmUAfGfCfmACfmAGf | 146 |
| | | mCmC | |
| PCSK9 | S | mC*mUmAmGmAmCCfmUGfmUdTmUmUmGmCmUmUmUm | 147 |
| | | UmGmU | |
| | AS | mA*Cf*mAAfAfAfmGCfmAAfmAAfmCAfmGGfmUCf | 148 |
| | | mUmAmG*mA*mA | |
| PCSK9 | S | mC*mU*mAmGmAmCCfmUGfmUdTmUmUmGmCmUmUmU | 149 |
| | | mUmGmU | |
| | AS | mA*mC*mAAfmAAfmGCfmAAfmAAfmCmAmGmGmUmC | 150 |
| | | mUmAmG*mA*mA | |
| PCSK9 | S | mC*mU*mAmGmAmCCfmUGfmUdTmUmUmGmCmUmUmU | 151 |
| | | mUmGmU | |
| | AS | mA*Cf*mAAfmAAfmGCfmAAfmAAfmCmAmGmGmUmCm | 152 |
| | | UmAmG*mA*mA | |
| PCSK9 | S | mC*mU*mAmGmAmCCfmUGfmUdTmUmUmGmCmUmUmU | 153 |
| | | mUmGmU | |
| | AS | mA*mC*mAAfmAAfmGCfmAAfmAAfmCAfmGmGmUmC | 154 |
| | | mUmAmG*mA*mA | |
| PCSK9 | S | mC*mU*mAmGmAmCCfmUGfmUdTmUmUmGmCmUmUmU | 155 |
| | | mUmGmU | |
| | AS | mA*mC*mAAfmAAfmGCfmAAfmAAfmCmAmGGfmUmC | 156 |
| | | mUmAmG*mA*mA | |
| PCSK9 | S | mC*mU*mAmGmAmCCfmUGfmUdTmUmUmGmCmUmUmU | 157 |
| | | mUmGmU | |
| | AS | mA*mC*mAAfmAAfmGCfmAAfmAAfmCmAmGmGmUCf | 158 |
| | | mUmAmG*mA*mA | |
| ANGPTL3 | S | mGmUmCmUmCmAAfmAAfmUGfmGmAmAmGmGmUmUmA | 159 |
| | | mUmA | |
| | AS | mUmAmUmAmACfmCUfUfCfmCAfmUUfmUUfmGmAmG | 160 |
| | | mAmCmUmU | |
| ANGPTL3 | S | mGmUmCmUmCmAAfmAAfmUdGmGmAmAmGmGmUmUmA | 161 |
| | | mUmA | |
| | AS | mUmAmUmAmACfmCUfUfCfmCAfmUUfmUUfmGmAmG | 162 |
| | | mAmCmUmU | |
| ANGPTL3 | S | mGmUmCmUmCmAmAAfmAUfdGmGmAmAmGmGmUmUmA | 163 |
| | | mUmA | |
| | AS | mUmAmUmAmACfmCUfUfCfmCAfmUUfmUUfmGmAmG | 164 |
| | | mAmCmUmU | |
| ANGPTL3 | S | mG*mU*mCmUmCmAAfmAAfmUdGmGmAmAmGmGmUmU | 165 |
| | | mAmUmA | |
| | AS | mUmAmUmAmACfmCUfUfCfmCAfmUUfmUUfmGmAmG | 166 |
| | | mAmCmUmU | |
| ANGPTL3 | S | mG*mU*mCmUmCmAmAAfmAUfdGmGmAmAmGmGmUmU | 167 |
| | | mAmUmA | |
| | AS | mUmAmUmAmACfmCUfUfCfmCAfmUUfmUUfmGmAmG | 168 |
| | | mAmCmUmU | |
| ANGPTL3 | S | mGmUmCmUmCmAAfmAAfmUdGmGmAmAmGmGmUmUmA | 169 |
| | | mUmA | |
| | AS | mU*mA*mUmAmACfmCUfUfCfmCAfmUUfmUUfmGmA | 170 |
| | | mGmAmC*mU*mU | |
| ANGPTL3 | S | mGmUmCmUmCmAAfmAAfmUdGmGmAmAmGmGmUmUmA | 171 |
| | | mUmA | |
| | AS | mUmAmUmAmACfmCUfUfCfCfAfmUUfmUUfmGmAmG | 172 |
| | | mAmCmUmU | |
| ANGPTL3 | S | mGmUmCmUmCmAAfmAAfmUdGmGmAmAmGmGmUmUmA | 173 |
| | | mUmA | |
| | AS | mU*mA*mUmAmACfmCUfUfCfmCAfmUUfmUUfmGmA | 174 |
| | | mGmAmC*mU*mU | |
| ANGPTL3 | S | mAmAmAmUmCmAmCGfmAmAdAmCmCAfmAmCmUmAmU | 175 |
| | AS | mAUfmAmGmUmUmGGfmUUfmUCfmGUfmGmAmUmUmU | 176 |
| | | CfmC | |
| ANGPTL3 | S | mAmAmAmUmCmAmCGfmAmAAfmCmCAfmAmCmUmAmU | 177 |
| | AS | mAUfmAmGmUmUmGGfmUUfmUCfmGUfmGmAmUmUmU | 178 |
| | | CfmC | |
| ANGPTL3 | S | mAmAmAmUmCmAmCGfmAAfdAmCmCAfmAmCmUmAmU | 179 |
| | AS | mAUfmAmGmUmUmGGfmUUfmUCfmGUfmGmAmUmUmU | 180 |
| | | CfmC | |
| ANGPTL3 | S | mAmAmAmUmCmACfmGAfmAdAmCmCAfmAmCmUmAmU | 181 |
| | AS | mAUfmAmGmUmUmGGfmUUfmUCfmGUfmGmAmUmUmU | 182 |
| | | CfmC | |
| ANGPTL3 | S | mAmAmAmUmCmAmCGfmAmAdAmCmCmAAfmCmUmAmU | 183 |
| | AS | mAUfmAmGmUmUmGGfmUUfmUCfmGUfmGmAmUmUmU | 184 |
| | | CfmC | |
| ANGPTL3 | S | mAmAmAmUmCmAmCGfmAmAdAmCmCmAmACfmUmAmU | 185 |
| | AS | mAUfmAmGmUmUmGGfmUUfmUCfmGUfmGmAmUmUmU | 186 |
| | | CfmC | |
| ANGPTL3 | S | mA*mA*mAmUmCmAmCGfmAmAdAmCmCAfmAmCmUmA | 187 |
| | | mU | |
| | AS | mAUfmAmGmUmUmGGfmUUfmUCfmGUfmGmAmUmUmU | 188 |
| | | CfmC | |
| ANGPTL3 | S | mAmAmAmUmCmAmCGfmAmAdAmCmCAfmAmCmUmAmU | 189 |
| | AS | mAUfmAmGmUmUmGmGUfmUUfCfmGUfmGmAmUmUmU | 190 |
| | | CfmC | |
| ANGPTL3 | S | mAmAmAmUmCmAmCGfmAmAdAmCmCAfmAmCmUmAmU | 191 |
| | AS | mAUfmAmGmUmUmGmGmUmUmUCfmGUfmGmAmUmUmU | 192 |
| | | CfmC | |
| ANGPTL3 | S | mAmAmAmUmCmAmCGfmAmAdAmCmCAfmAmCmUmAmU | 193 |
| | AS | mAUfmAmGmUmUmGGfmUUfmUCfmGUfmGmAmUmUmU | 194 |
| | | CfmC | |
| ANGPTL3 | S | mAmAmAmUmCmAmCGfmAmAdAmCmCAfmAmCmUmAmU | 195 |
| | AS | mA*Uf*mAmGmUmUmGGfmUUfmUCfmGUfmGmAmUmU | 196 |
| | | mU*Cf*mC | |
| ANGPTL3 | S | mAmAmAmUmCmAmCGfmAmAdAmCmCAfmAmCmUmAmU | 197 |
| | AS | mA*Uf*mAmGmUmUmGGfmUUfmUCfmGUfmGmAmUmU | 198 |
| | | mUCf*mC | |
| ANGPTL3 | S | mAmAmAmUmCmAmCGfmAmAdAmCmCAfmAmCmUmAmU | 199 |
| | AS | mA*UfmAmGmUmUmGGfmUUfmUCfmGUfmGmAmUmUm | 200 |
| | | U*Cf*mC | |
| ANGPTL3 | S | mAmAmAmUmCmAmCGfmAmAdAmCmCAfmAmCmUmAmU | 201 |
| | AS | mA*UfmAmGmUmUmGGfmUUfmUCfmGUfmGmAmUmUm | 202 |
| | | UCf*mC | |
| ANGPTL3 | S | mAmAmAmUmCmACfmGAfmAdAmCmCAfmAmCmUmAmU | 203 |
| | AS | mA*Uf*mAmGmUmUmGGfmUUfmUCfmGUfmGmAmUmU | 204 |
| | | mU*Cf*mC | |
| ANGPTL3 | S | mAmAmAmUmCmAmCGfmAmAdAmCmCmAmACfmUmAmU | 205 |
| | AS | mAUfmAmGmUmUmGGfmUUfmUCfmGUfmGmAmUmUmU | 206 |
| | | CfmC | |
| ANGPTL3 | S | mA*mA*mAmUmCmAmCGfmAmAdAmCmCAfmAmCmUmA | 207 |
| | | mU | |
| | AS | mAUfmAmGmUmUmGGfmUUfmUCfmGUfmGmAmUmUmU | 208 |
| | | CfmC | |
| ANGPTL3 | S | mAmAmAmUmCmAmCGfmAmAdAmCmCAfmAmCmUmAmU | 209 |
| | AS | mAUfmAmGmUmUmGmGUfmUUfCfmGUfmGmAmUmUmU | 210 |
| | | CfmC | |
| ANGPTL3 | S | mAmAmAmUmCmAmCGfmAmAdAmCmCAfmAmCmUmAmU | 211 |
| | AS | mAUfmAmGmUmUmGmGmUmUmUCfmGUfmGmAmUmUmU | 212 |
| | | CfmC | |
| ANGPTL | S | mAmAmAmUmCmAmCGfmAmAdAmCmCAfmAmCmUmAmU | 213 |
| | AS | mAUfmAmGmUmUmGGfmUUfmUCfmGUfmGmAmUmUmU | 214 |
| | | CfmC | |
| ANGPTL3 | S | mAmAmAmUmCmAmCGfmAmAdAmCmCAfmAmCmUmAmU | 215 |
| | AS | mA*Uf*mAmGmUmUmGGfmUUfmUCfmGUfmGmAmUmU | 216 |
| | | mU*Cf*mC | |
| ANGPTL3 | S | mA*mAmAmUmCmACfmGAfmAdAmCmCAfmAmCmUmAm | 217 |
| | | U | |
| | AS | mA*Uf*mAmGmUmUmGGfmUUfmUCfmGUfmGmAmUmU | 218 |
| | | mU*Cf*mC | |
| ANGPTL3 | S | mAmA*mAmUmCmACfmGAfmAdAmCmCAfmAmCmUmAm | 219 |
| | | U | |
| | AS | mA*Uf*mAmGmUmUmGGfmUUfmUCfmGUfmGmAmUmU | 220 |
| | | mU*Cf*mC | |
| ANGPTL3 | S | mA*mA*mAmUmCmACfmGAfmAdAmCmCAfmAmCmUmA | 221 |
| | | mU | |
| | AS | mA*Uf*mAmGmUmUmGGfmUUfmUCfmGUfmGmAmUmU | 222 |
| | | mU*Cf*mC | |
| ANGPTL3 | S | mAmAmAmUmCmACfmGAfmAdAmCmCAfmAmCmUmAmU | 223 |
| | AS | mA*Uf*mAmGmUmUmGGfmUUfmUmCmGUfmGmAmUmU | 224 |
| | | mU*Cf*mC | |
| ANGPTL3 | S | mGmUmCmUmCmAAfmAAfmUdGmGmAmAmGmGmUmUmA | 225 |
| | | mUmA | |
| | AS | mU*mA*mUmAmACfmCUfUfCfmCAfmUUfmUUfmGmA | 226 |
| | | mGmAmC*mU*mU | |
| ANGPTL3 | S | mG*mUmCmUmCmAAfmAAfmUdGmGmAmAmGmGmUmUm | 227 |
| | | AmUmA | |
| | AS | mU*mA*mUmAmACfmCUfUfCfmCAfmUUfmUUfmGmA | 228 |
| | | mmGmAmC*mU*mU | |
| ANGPTL3 | S | mGmU*mCmUmCmAAfmAAfmUdGmGmAmAmGmGmUmUm | 229 |
| | | AmUmA | |
| | AS | mU*mA*mUmAmACfmCUfUfCfmCAfmUUfmUUfmGmA | 230 |
| | | mGmAmC*mU*mU | |
| ANGPTL3 | S | mG*mU*mCmUmCmAAfmAAfmUdGmGmAmAmGmGmUmU | 231 |
| | | mAmUmA | |
| | AS | mU*mA*mUmAmACfmCUfUfCfmCAfmUUfmUUfmGmA | 232 |
| | | mGmAmC*mU*mU | |
| ANGPTL3 | S | mGmUmCmUmCmAAfmAAfmUdGfGmAmAmGmGmUmUmA | 233 |
| | | mUmA | |
| | AS | mU*mA*mUmAmACfmCUfUfCfmCAfmUUfmUUfmGmA | 234 |
| | | mGmAmC*mU*mU | |
| ANGPTL3 | S | mGmUmCmUmCmAAfmAAfmUdGmGfAmAmGmGmUmUmA | 235 |
| | | mUmA | |
| | AS | mU*mA*mUmAmACfmCUfUfCfmCAfmUUfmUUfmGmA | 236 |
| | | mGmAmC*mU*mU | |
| ANGPTL3 | S | mGmUmCmUmCmAAfmAAfmUdGmGmAmAmGmGmUmUmA | 237 |
| | | mUmA | |
| | AS | mU*mA*mUmAmACfmCUfUfCfmCAfmUUfmUUfmGmA | 238 |
| | | mGmAmC*mU*mU | |

In some embodiments of conjugates of the disclosure, the bioactive agent is a PCSK9-siRNA. In such embodiments, the conjugate is useful for treating or preventing a PCSK9-related disease or disorder. PSCK9-related diseases and disorders include, for example and without limitation, atherosclerosis, hypercholesterolemia, acute coronary syndrome, dyslipidemia, myocardial infarction, coronary artery disease, stroke, coronary artery disease, cardiovascular disease, diabetes, hyperlipidemia, type two diabetes mellitus, and kidney disease, as described further herein.

In one embodiment of conjugates of the disclosure, the bioactive agent is an siRNA having the sequence set forth in SEQ ID NO: 147/148. The specific sequence is the following:

Sense:
(SEQ ID NO: 147)
5′-mC*mUmAmGmAmCCfmUGfmUdTmUmUmGmCmUmUmUmUmGmU-3′;
and
Antisense:
(SEQ ID NO: 148)
5′-mA*Cf*mAAfAfAfmGCfmAAfmAAfmCAfmGGfmUCfmUmAmG*m
A*mA-3′.

In one embodiment of conjugates of the disclosure, the bioactive agent is an siRNA comprising or consisting of the sequence set forth in SEQ ID NO: 147/148, i.e., comprising or consisting of the following:

Sense:
(SEQ ID NO: 147)
5′-mC*mUmAmGmAmCCfmUGfmUdTmUmUmGmCmUmUmUmUmGmU-3′;
and
Antisense:
(SEQ ID NO: 148)
5′-mA*Cf*mAAfAfAfmGCfmAAfmAAfmCAfmGGfmUCfmUmAmG*m
A*mA-3′.

In some embodiments of conjugates of the disclosure, the bioactive agent is an ANGPTL3-siRNA. In such embodiments, the conjugate is useful for treating or preventing an ANGPTL3-related disease or disorder. ANGPTL3-related diseases and disorders include, for example and without limitation, atherosclerosis, hypercholesterolemia, acute coronary syndrome, dyslipidemia, myocardial infarction, coronary artery disease, stroke, coronary artery disease, cardiovascular disease, diabetes, hyperlipidemia, type 2 diabetes, and kidney disease, as described further herein.

In one embodiment of conjugates of the disclosure, the bioactive agent is an siRNA having the sequence set forth in SEQ ID NO: 179/180. The specific sequence is the following:

Sense:
(SEQ ID NO: 179)
5′-mAmAmAmUmCmAmCGfmAAfdAmCmCAfmAmCmUmAmU-3′;
and
Antisense:
(SEQ ID NO: 180)
5′-mAUfmAmGmUmUmGGfmUUfmUCfmGUfmGmAmUmUmUCfmC-3′.

In one embodiment of conjugates of the disclosure, the bioactive agent is an siRNA comprising or consisting of the sequence set forth in SEQ ID NO: 179/180, i.e., comprising or consisting of the following:

Sense:
(SEQ ID NO: 179)
5′-mAmAmAmUmCmAmCGfmAAfdAmCmCAfmAmCmUmAmU-3′;
and
Antisense:
(SEQ ID NO: 180)
5′-mAUfmAmGmUmUmGGfmUUfmUCfmGUfmGmAmUmUmUCfmC-3′.

In some embodiments of conjugates of the disclosure, the bioactive agent is an Apolipoprotein B (ApoB)-siRNA. In such embodiments, the conjugate is useful for treating or preventing an ApoB-related disease or disorder. ApoB-related diseases and disorders include, for example and without limitation, atherosclerosis, hypercholesterolemia, acute coronary syndrome, dyslipidemia, myocardial infarction, coronary artery disease, stroke, coronary artery disease, cardiovascular disease, diabetes, hyperlipidemia, type 2 diabetes, and kidney disease.

In some embodiments of conjugates of the disclosure, the bioactive agent is an Factor VII (FVII)-siRNA. In such embodiments, the conjugate is useful for treating or preventing an FVII-related disease or disorder. FVII-related diseases and disorders include, for example and without limitation, anemia, coagulation factor deficiency, hemophilia, liver disease, vitamin K deficiency, thrombosis and hemorrhage.

In a third broad aspect, there are provided pharmaceutical compositions comprising a compound of the disclosure (a conjugate and/or bivalent compound), or a pharmaceutically acceptable salt or ester thereof, and a pharmaceutically acceptable carrier.

In some embodiments, there are provided pharmaceutical compositions comprising a conjugate as described herein, or a pharmaceutically acceptable salt or ester thereof, and a pharmaceutically acceptable carrier. In some such embodiments, the conjugate comprises a bioactive agent which is an RNAi agent (e.g., an siRNA, e.g., a double stranded RNA) and is administered in an unbuffered solution, such as, for example and without limitation, saline or water. In other embodiments, the conjugate comprises a bioactive agent which is an RNAi agent (e.g., an siRNA, e.g., a double stranded RNA) and is administered with a buffer solution. The buffer solution may comprise, for example and without limitation, acetate, citrate, prolamine, carbonate, phosphate, or a combination thereof. In an embodiment, the buffer solution is phosphate buffered saline (PBS).

In some embodiments, the pharmaceutical composition is in a form suitable for parenteral administration, for example subcutaneously, intramuscularly, intravenously or intraperitoneally, for example in the form of solutions for injection or infusion. In some embodiments, the pharmaceutical composition is in a form suitable for subcutaneous administration. In some embodiments, the pharmaceutical composition is in a form suitable for intravenous administration. In other embodiments, the pharmaceutical composition is in a form suitable for oral, topical, transdermal, mucosal, or nasal administration. Pharmaceutically acceptable carriers may include, for example and without limitation, creams, emulsions, gels, liposomes, or nanoparticles, as discussed further hereinbelow.

In some embodiments, pharmaceutical compositions of the disclosure are useful for the treatment and/or prevention of diseases or disorders for which the bioactive agent(s) has a corresponding therapeutic and/or prophylactic effect. Pharmaceutical compositions of the disclosure can act to improve delivery of the bioactive agent to the target site of interest and thereby improve therapeutic efficacy of the bioactive agent(s). For example, in embodiments where the bioactive agent is an siRNA directed against PCSK9 or ANGPTL3, the pharmaceutical composition is useful for the treatment and/or prevention of PCSK9 or ANGPTL3 related diseases or disorders, respectively.

In a fourth broad aspect, there are provided methods of inhibiting expression of a target gene in a cell, the method comprising contacting the cell with a compound of the disclosure (a conjugate and/or a bivalent compound) or a pharmaceutical composition thereof, and maintaining the cell for a time sufficient to inhibit expression of the target gene in the cell. In some such embodiments, the cell is maintained for a time sufficient to obtain degradation of the mRNA transcript of the target gene, thereby inhibiting expression of the target gene in the cell. In some embodiments, the cell is within a subject, e.g., a human subject. In some embodiments, the expression of the target gene is inhibited by at least about 30%, at least about 40%, at least about 50%, at least about 60%, at least about 70%, or at least about 80%.

In some embodiments, the cell is a liver cell.

In some embodiments, the target gene is PCSK9.

In some embodiments, the subject has a disorder mediated by PCSK9 expression or a PCSK9-related disease or disorder.

In some embodiments, there are provided methods of treating a subject having a PCSK9-related disease or disorder or a disorder mediated by PCSK9 expression, comprising administering to the subject a therapeutically effective amount of a compound of the disclosure or a pharmaceutical composition thereof, such that the subject is treated. In some such embodiments, the subject has hypercholesterolemia, dyslipidemia, or hyperlipidemia. In some such embodiments, the subject has atherosclerotic cardiovascular disease (e.g., ASCVD). In some such embodiments, the subject has a metabolic disorder such as diabetes, e.g., type 2 diabetes. In some embodiments, serum cholesterol levels and/or low-density lipoprotein cholesterol (LDL-C) levels are reduced in the subject after administration of the compound or composition described herein.

In some embodiments, there are provided methods of treating or preventing a PCSK9-related disease or disorder in a subject, comprising administering to the subject a therapeutically effective amount of a compound of the disclosure (or a pharmaceutically acceptable salt or ester thereof) or a pharmaceutical composition thereof, such that the PCSK9-related disease or disorder is treated or prevented in the subject. Non-limiting examples of PCSK9-related diseases and disorders include atherosclerosis, hypercholesterolemia (e.g., heterozygous familial hypercholesterolemia (HeFH), primary hypercholesterolaemia), acute coronary syndrome, dyslipidemia (e.g., mixed dyslipidaemia), myocardial infarction, coronary artery disease, stroke, coronary artery disease, cardiovascular disease, diabetes, hyperlipidemia, type 2 diabetes, and kidney disease. In an embodiment, a PCSK9-related disease or disorder is a hepatic disease or disorder, or a disease or disorder for which targeting to the liver is beneficial. In an embodiment, a PCSK9-related disease or disorder is hypercholesterolemia, dyslipidemia, or hyperlipidemia. In an embodiment, a PCSK9-related disease or disorder is atherosclerotic cardiovascular disease (ASCVD). In an embodiment, a PCSK9-related disease or disorder is an ASCVD risk equivalent.

In some embodiments, there are provided methods of treating or preventing a ANGPTL3-related disease or disorder in a subject, comprising administering to the subject a therapeutically effective amount of a compound of the disclosure (or a pharmaceutically acceptable salt or ester thereof) or a pharmaceutical composition thereof, such that the ANGPTL3-related disease or disorder is treated or prevented in the subject. In some such embodiments, the ANGPTL3-related disease or disorder is atherosclerosis, hypercholesterolemia, acute coronary syndrome, dyslipidemia, myocardial infarction, coronary artery disease, stroke, cardiovascular disease, diabetes, hyperlipidemia, type 2 diabetes, or kidney disease.

In some embodiments, there are provided methods of treating or preventing hypercholesterolemia in a subject, the method comprising administering to the subject a therapeutically effective amount of a compound of the disclosure (or a pharmaceutically acceptable salt or ester thereof) or a pharmaceutical composition thereof, such that hypercholesterolemia is treated or prevented in the subject. Hypercholesterolemia may be, for example and without limitation, familial hypercholesterolemia, such as heterozygous familial hypercholesterolemia, or non-familial hypercholesterolemia, such as hypercholesterolemia associated with atherosclerotic or cardiovascular disease.

In some embodiments, there are provided methods of treating or preventing atherosclerotic cardiovascular disease in a subject, the methods comprising administering to the subject a therapeutically effective amount of a compound of the disclosure (or a pharmaceutically acceptable salt or ester thereof) or a pharmaceutical composition thereof, such that atherosclerotic cardiovascular disease is treated or prevented in the subject. In some embodiments, the atherosclerotic cardiovascular disease is ASCVD.

In some embodiments, there are provided methods of treating or preventing type 2 diabetes in a subject, the methods comprising administering to the subject a therapeutically effective amount of a compound of the disclosure (or a pharmaceutically acceptable salt or ester thereof) or a pharmaceutical composition thereof, such that the type 2 diabetes is treated or prevented in the subject.

In some embodiments, there are provided methods for reducing serum cholesterol level in a subject, the methods comprising administering to the subject a therapeutically effective amount of a compound of the disclosure (or a pharmaceutically acceptable salt or ester thereof) or a pharmaceutical composition thereof, such that serum cholesterol level is reduced in the subject. In some embodiments, the subject suffers from hypercholesterolemia, dyslipidemia, hyperlipidemia, atherosclerotic cardiovascular disease, or type 2 diabetes.

In some embodiments, there are provided methods for reducing low-density lipoprotein cholesterol (LDL-C) level in a subject, the methods comprising administering to the subject a therapeutically effective amount of a compound of the disclosure (or a pharmaceutically acceptable salt or ester thereof) or a pharmaceutical composition thereof, such that LDL-C level is reduced in the subject. In some embodiments, the subject suffers from hypercholesterolemia, dyslipidemia, hyperlipidemia, atherosclerotic cardiovascular disease, or type 2 diabetes.

In some embodiments of therapeutic and prophylactic methods provided herein, the methods further comprise a step of determining an LDL receptor (LDLR) genotype or phenotype of the subject.

In some embodiments of therapeutic and prophylactic methods provided herein, the methods further comprise a step of determining the serum cholesterol level in the subject. The serum cholesterol level may be determined before, during, and/or after said administering.

In some embodiments of therapeutic and prophylactic methods provided herein, the methods further comprise a step of determining the LDL-C level in the subject. The LDL-C level may be determined before, during, and/or after said administering.

In some embodiments of therapeutic and prophylactic methods provided herein, administration of the compound or composition of the disclosure acts to reduce serum cholesterol levels and/or LDL-C levels in the subject.

In some embodiments of therapeutic and prophylactic methods provided herein, the subject is a mammal, e.g., a primate, a rodent, or a human. In one embodiment, the subject is a human.

In some embodiments of therapeutic and prophylactic methods provided herein, the compound is administered at a dose of about 0.01 mg/kg to about 10 mg/kg, of about 0.5 mg/kg to about 50 mg/kg, or of about 10 mg/kg to about 30 mg/kg. In some embodiments of therapeutic and prophylactic methods provided herein, the compound is administered at a dose of from about 50 mg to about 500 mg, or from about 200 mg to about 300 mg, or of about 50 mg, about 100 mg, about 200 mg, about 300 mg, about 400 mg, or about 500 mg.

In some embodiments of therapeutic and prophylactic methods provided herein, the compound or the composition is administered in two or more doses. In some such embodiments, the compound or the composition is administered in a dosing regimen that includes a loading phase followed by a maintenance phase, wherein the loading phase comprises administering a dose of 2 mg/kg, 1 mg/kg or 0.5 mg/kg five times a week, and wherein the maintenance phase comprises administering a dose of 2 mg/kg, 1 mg/kg or 0.5 mg/kg once a week, twice a week, three times a week, once every two weeks, once every three weeks, once a month, once every two months, once every three months, once every four months, once every five months, or once every six months. In other embodiments, the compound or the composition described herein is administered at intervals of once every about 12 hours, once every about 24 hours, once every about 48 hours, once every about 72 hours, or once every about 96 hours.

In some embodiments of therapeutic and prophylactic methods provided herein, the administration of the compound or composition described herein results in a decrease in serum cholesterol, e.g., a reduced or lowered level of serum cholesterol, in the subject. In some embodiments of therapeutic and prophylactic methods provided herein, the administration of the compound or composition described herein results in a decrease in LDL-C, e.g., a reduced or lowered level of LDL-C, in the subject.

In some embodiments of therapeutic and prophylactic methods provided herein, the compound or the composition is administered parenterally, for example subcutaneously, intramuscularly, intravenously or intraperitoneally in the form of solutions for injection or infusion. In one embodiment, the compound or the composition is administered subcutaneously. In one embodiment, the compound or the composition is administered intravenously.

In some embodiments, there are provided therapeutic and/or prophylactic uses of the compounds or compositions of the disclosure, as described herein. Use of the compounds or compositions of the disclosure in the manufacture of a medicament for the treatment or prevention of a disease or disorder are also provided.

In another broad aspect, there are provided kits comprising one or more compound of the disclosure or pharmaceutical composition thereof, as described herein. A kit may further comprise one or more additional therapeutic agents and/or instructions, for example, instructions for using the kit to treat a subject having a disease or disorder targeted by the compound of the disclosure.

### BRIEF DESCRIPTION OF THE DRAWINGS

For a better understanding of the invention and to show more clearly how it may be carried into effect, reference will now be made by way of example to the accompanying drawings, which illustrate aspects and features according to embodiments of the present invention, and in which:

FIGS. 1A-1E are graphs showing humanized PCSK9 protein level vs. time in humanized PCSK9 mice after treatment with a conjugate of the disclosure or control, as indicated: I: Control (saline); II: Positive control (Inclisiran); A: conjugate 1; B: conjugate 2; C: conjugate 3; D: conjugate 4; and E: conjugate 5. Error bars represent standard error of the mean (SEM), n=6.

### DETAILED DESCRIPTION

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

The term “or” is used herein to mean, and is used interchangeably with, the term “and/or”, unless context clearly indicates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) and “containing” (and any form of containing, such as “contain” and “contains”), are inclusive or open-ended and do not exclude additional, unrecited elements or process steps.

The term “about” is used herein to indicate that a value includes an inherent variation of error for the device or the method being employed to determine the value.

The term “derivative” as used herein, is understood as being a substance similar in structure to another compound but differing in some slight structural detail.

As used herein, the term “administration” or “administered” refers to delivering a compound to a subject, including all the means of dosing and drug delivery known in the art.

### Compounds

As used herein, the terms “compounds of the disclosure”, “compounds of the present disclosure” and equivalent expressions refer to bivalent compounds and conjugates described herein as being useful for at least one purpose described herein, and including specific bivalent compounds and conjugates described herein, as well as their pharmaceutically acceptable salts and esters. It should be understood that compounds of the disclosure may be further substituted with one or more substituting group when such a substitution is available, as will be understood by the skilled artisan. In some embodiments, a substituted form of a compound is a prodrug; in such embodiments, the substituent can be cleaved, or the compound can be otherwise converted, to release the active component or the drug compound from the prodrug form after being administered to a subject.

As used herein, the term “substituted” or “with substitution” refers to a compound or a moiety having at least one (1) substituent group. The term “unsubstituted” or “without substitution” refers to a compound or a moiety having no other substituent group except that the unidentified valence is chemically saturated with hydrogen atoms.

As used herein, a “substituent” or a “substituent group” refers to a group selected from halogen (F, Cl, Br, or I), hydroxy, sulfhydryl, amino, nitro, carbonyl, carboxyl, alkyl, alkoxyl, alkylamino, aryl, aryloxyl, arylamino, acyl, thionyl, sulfonyl, phosphonyl, and other organic moiety as used and accepted in general organic chemistry.

As used herein, the term “alkyl” refers to saturated hydrocarbons having from one to twelve carbon atoms, including linear, branched, and cyclic alkyl groups. Examples of alkyl groups include, without limitation, methyl, ethyl, propyl, butyl, pentyl, hexyl, heptyl, octyl, nonyl, decyl, isopropyl, tert-butyl, sec-butyl, isobutyl, cyclopropyl, cyclopentyl, cyclohexyl, cycloheptyl, cyclooctyl, and the like. The term alkyl includes both unsubstituted alkyl groups and substituted alkyl groups. The term “C1-Cnalkyl”, wherein n is an integer from 2 to 12, refers to an alkyl group having from 1 to the indicated “n” number of carbon atoms. Alkyl residues may be substituted or unsubstituted. In some embodiments, for example, alkyl may be substituted by hydroxyl, amino, carboxyl, carboxylic ester, amide, carbamate, or aminoalkyl.

Unless the number of carbons is otherwise specified, “lower” as in “lower aliphatic,” “lower alkyl,” “lower alkenyl,” and “lower alkylnyl”, as used herein means that the moiety has at least one (two for alkenyl and alkynyl) and equal to or less than 6 carbon atoms.

The terms “cycloalkyl”, “alicyclic”, “carbocyclic” and equivalent expressions refer to a group comprising a saturated or partially unsaturated carbocyclic ring in a single, spiro (sharing one atom), or fused (sharing at least one bond) carbocyclic ring system having from three to fifteen ring members. Examples of cycloalkyl groups include, without limitation, cyclopropyl, cyclobutyl, cyclopentyl, cyclopenten-1-yl, cyclopenten-2-yl, cyclopenten-3-yl, cyclohexyl, cyclohexen-1-yl, cyclohexen-2-yl, cyclohexen-3-yl, cycloheptyl, bicyclo[4,3,0]nonanyl, norbornyl, and the like. The term cycloalkyl includes both unsubstituted cycloalkyl groups and substituted cycloalkyl groups. The term “C3-Cncycloalkyl”, wherein n is an integer from 4 to 15, refers to a cycloalkyl group having from 3 to the indicated “n” number of carbon atoms in the ring structure. Unless the number of carbons is otherwise specified, “lower cycloalkyl” groups as herein used, have at least 3 and equal to or less than 8 carbon atoms in their ring structure.

Cycloalkyl residues can be saturated or contain one or more double bonds within the ring system. In particular they can be saturated or contain one double bond within the ring system. In unsaturated cycloalkyl residues the double bonds can be present in any suitable positions. Monocycloalkyl residues are, for example, cyclopropyl, cyclobutyl, cyclopentyl, cyclopentenyl, cyclohexyl, cyclohexenyl, cycloheptyl, cycloheptenyl, cyclooctyl, cyclononyl, cyclodecyl, cycloundecyl, cyclododecyl or cyclotetradecyl, which can also be substituted, for example by C1-4 alkyl. Examples of substituted cycloalkyl residues are 4-methylcyclohexyl and 2,3-dimethylcyclopentyl. Examples of structures of bicyclic ring systems are norbomane, bicyclo[2.2.1]heptane, bicyclo[2.2.2]octane and bicyclo[3.2.1]octane.

The term “heterocycloalkyl” and equivalent expressions refer to a group comprising a saturated or partially unsaturated carbocyclic ring in a single, spiro (sharing one atom), or fused (sharing at least one bond) carbocyclic ring system having from three to fifteen ring members, including one to six heteroatoms (e.g., N, O, S, P) or groups containing such heteroatoms (e.g., NH, NRx (Rx is alkyl, acyl, aryl, heteroaryl or cycloalkyl), P02, SO, SO2, and the like). Heterocycloalkyl groups may be C-attached or heteroatom-attached (e.g., via a nitrogen atom) where such is possible. Examples of heterocycloalkyl groups include, without limitation, pyrrolidino, tetrahydrofuranyl, tetrahydrodithienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3,1,0]hexanyl, 3-azabicyclo[4,1,0]heptanyl, 3H-indolyl, quinolizinyl, and sugars, and the like. The term heterocycloalkyl includes both unsubstituted heterocycloalkyl groups and substituted heterocycloalkyl groups. The term “C3-Cnheterocycloalkyl”, wherein n is an integer from 4 to 15, refers to a heterocycloalkyl group having from 3 to the indicated “n” number of atoms in the ring structure, including at least one hetero group or atom as defined above. Unless the number of carbons is otherwise specified, “lower heterocycloalkyl” groups as herein used, have at least 3 and equal to or less than 8 carbon atoms in their ring structure.

The terms “aryl” and “aryl ring” refer to aromatic groups having “4n+2”π (pi) electrons, wherein n is an integer from 1 to 3, in a conjugated monocyclic or polycyclic system (fused or not) and having six to fourteen ring atoms. A polycyclic ring system includes at least one aromatic ring. Aryl may be directly attached, or connected via a C1-C₃alkyl group (also referred to as arylalkyl or aralkyl). Examples of aryl groups include, without limitation, phenyl, benzyl, phenetyl, 1-phenylethyl, tolyl, naphthyl, biphenyl, terphenyl, indenyl, benzocyclooctenyl, benzocycloheptenyl, azulenyl, acenaphthylenyl, fluorenyl, phenanthrenyl, anthracenyl, and the like. The term aryl includes both unsubstituted aryl groups and substituted aryl groups. The term “C6-Cnaryl”, wherein n is an integer from 6 to 15, refers to an aryl group having from 6 to the indicated “n” number of atoms in the ring structure, including at least one hetero group or atom as defined above.

The terms “heteroaryl” and “heteroaryl ring” refer to an aromatic groups having “4n+2”.pi.(pi) electrons, wherein n is an integer from 1 to 3, in a conjugated monocyclic or polycyclic system (fused or not) and having five to fourteen ring members, including one to six heteroatoms (e.g. N, O, S) or groups containing such heteroatoms (e.g. NH, NRx (Rx is alkyl, acyl, aryl, heteroaryl or cycloalkyl), SO, and the like). A polycyclic ring system includes at least one heteroaromatic ring. Heteroaryls may be directly attached, or connected via a C1-C3alkyl group (also referred to as heteroarylalkyl or heteroaralkyl). Heteroaryl groups may be C-attached or heteroatom-attached (e.g., via a nitrogen atom), where such is possible. Examples of heteroaryl groups include, without limitation, pyridyl, imidazolyl, pyrimidinyl, pyrazolyl, triazolyl, tetrazolyl, furyl, thienyl; isooxazolyl, thiazolyl, oxazolyl, isothiazolyl, pyrrollyl, quinolinyl, isoquinolinyl, indolyl, isoindolyl, chromenyl, isochromenyl, benzimidazolyl, benzofuranyl, cinnolinyl, indazolyl, indolizinyl, phthalazinyl, pyridazinyl, pyrazinyl, triazinyl, isoindolyl, pteridinyl, purinyl, oxadiazolyl, thiadiazolyl, furazanyl, benzofurazanyl, benzothiophenyl, benzothienyl, benzothiazolyl, benzoxazolyl, quinazolinyl, quinolizinyl, quinolonyl, isoquinolonyl, quinoxalinyl, naphthyridinyl, furopyridinyl, carbazolyl, phenanthridinyl, acridinyl, perimidinyl, phenanthrolinyl, phenazinyl, phenothiazinyl, phenoxazinyl, dibenzofurnayl, and the like. The term heteroaryl includes both unsubstituted heteroaryl groups and substituted heteroaryl groups. The term “C5-Cnheteroaryl”, wherein n is an integer from 6 to 15, refers to a heteroaryl group having from 5 to the indicated “n” number of atoms in the ring structure, including at least one hetero group or atom as defined above.

The terms “heterocycle” or “heterocyclic” include heterocycloalkyl and heteroaryl groups. Examples of heterocycles include, without limitation, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4αH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl, and the like. The term heterocycle includes both unsubstituted heterocyclic groups and substituted heterocyclic groups.

The term “amine” or “amino,” as used herein, refers to an unsubstituted or substituted moiety of the formula —NRaRb, in which Ra and Rb are each independently hydrogen, alkyl, aryl, or heterocyclyl, or Ra and Rb, taken together with the nitrogen atom to which they are attached, form a heterocyclic ring. The term amino includes compounds or moieties in which a nitrogen atom is covalently bonded to at least one carbon or heteroatom. Thus, the terms “alkylamino” and “dialkylamino” as used herein mean an amine group having respectively one and at least two C1-C₆alkyl groups attached thereto. The terms “acylamino” and “diarylamino” include groups wherein the nitrogen is bound to at least one or two aryl groups, respectively. The terms “amide” or “aminocarbonyl” include compounds or moieties which contain a nitrogen atom which is bound to the carbon of a carbonyl or a thiocarbonyl group. The term “acylamino” refers to an amino group directly attached to an acyl group as defined herein.

The term “alkylthio” refers to an alkyl group, having a sulfhydryl group attached thereto. Suitable alkylthio groups include groups having 1 to about 12 carbon atoms, preferably from 1 to about 6 carbon atoms. The term “alkylcarboxyl” as used herein means an alkyl group having a carboxyl group attached thereto.

The terms “alkoxy” or “lower alkoxy” as used herein mean an alkyl group having an oxygen atom attached thereto. Representative alkoxy groups include groups having 1 to about 6 carbon atoms, e.g., methoxy, ethoxy, propoxy, tert-butoxy and the like. Examples of alkoxy groups include methoxy, ethoxy, isopropyloxy, propoxy, butoxy, pentoxy, fluoromethoxy, difluoromethoxy, trifluoromethoxy, chloromethoxy, dichloromethoxy, trichloromethoxy groups, and the like. The term “alkoxy” includes both unsubstituted or substituted alkoxy groups, etc., as well as perhalogenated alkyloxy groups.

The terms “carbonyl” or “carboxy” include compounds and moieties which contain a carbon connected with a double bond to an oxygen atom. Examples of moieties which contain a carbonyl include aldehydes, ketones, carboxylic acids, amides, esters, anhydrides, etc.

The term “acyl” refers to a carbonyl group that is attached through its carbon atom to a hydrogen (i.e., formyl), an aliphatic group (C1-C₆alkyl, C1-C₆alkenyl, C1-C₆alkynyl, e.g., acetyl), a cycloalkyl group (C3-C8cycloalkyl), a heterocyclic group (C3-C8 heterocycloalkyl and C5-C6 heteroaryl), an aromatic group (C6 aryl, e.g., benzoyl), and the like. Acyl groups may be unsubstituted or substituted acyl groups (e.g., salicyloyl).

For compounds of the disclosure, it is intended that, in some embodiments, esters thereof are also encompassed. The term “ester” refers to compounds that can be represented by the formula RCOOR (carboxylic ester) or the formula RSO₃R′ (sulfonate ester), usually respectively formed by the reaction between a carboxylic or a sulfonic acid and an alcohol usually with the elimination of water.

For compounds of the disclosure, it is intended that, in some embodiments, salts thereof are also encompassed, including pharmaceutically acceptable salts. Those skilled in the art will appreciate that many salt forms (e.g., TFA salt, tetrazolium salt, sodium salt, potassium salt, etc,) are possible; appropriate salts are selected based on considerations known in the art. The term “pharmaceutically acceptable salt” refers to salts prepared from pharmaceutically acceptable non-toxic acids or bases including inorganic acids and bases and organic acids and bases. For example, for compounds that contain a basic nitrogen, salts may be prepared from pharmaceutically acceptable non-toxic acids including inorganic and organic acids. Suitable pharmaceutically acceptable acid addition salts for the compounds provided herein include without limitation acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds provided herein include without limitation metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine.

In some embodiments, a compound of the disclosure includes a bivalent compound of Formula (I), (II), (III), or (IV), or a pharmaceutically acceptable salt or ester thereof. In some embodiments, a compound of the disclosure includes a conjugate of Formula (V), or a pharmaceutically acceptable salt or ester thereof. In some embodiments, a compound of the disclosure includes a conjugate shown in Table 1, or a pharmaceutically acceptable salt or ester thereof. In some embodiments, a compound of the disclosure includes a bioactive agent shown in Table 2 or Table 3. In some embodiments, a compound of the disclosure comprises or consists of conjugate 5, or a pharmaceutically acceptable salt or ester thereof. In some embodiments, a compound of the disclosure comprises or consists of conjugate 1, or a pharmaceutically acceptable salt or ester thereof. In some embodiments, a compound of the disclosure comprises or consists of conjugate 2, or a pharmaceutically acceptable salt or ester thereof. In some embodiments, a compound of the disclosure comprises or consists of conjugate 3, or a pharmaceutically acceptable salt or ester thereof. In some embodiments, a compound of the disclosure comprises or consists of conjugate 4, or a pharmaceutically acceptable salt or ester thereof. In some such embodiments, the bioactive agent is an siRNA comprising or consisting of the sequence shown in SEQ ID NO: 1/2. In some such embodiments, the bioactive agent is an siRNA comprising or consisting of the sequence shown in SEQ ID NO: 5/6. In some such embodiments, the bioactive agent is an siRNA comprising or consisting of the sequence shown in SEQ ID NO: 147/148. In some such embodiments, the bioactive agent is an siRNA comprising or consisting of the sequence shown in SEQ ID NO: 179/180.

A “pharmaceutically acceptable salt” of a compound means a salt of a compound that is pharmaceutically acceptable. Desirable are salts of a compound that retain or improve the biological effectiveness and properties of the free acids and bases of the same compound as defined herein or that take advantage of an intrinsically basic, acidic or charged functionality in the compound and that are not biologically or otherwise undesirable. Examples of pharmaceutically acceptable salts are also described, for example, in Berge et al., “Pharmaceutical Salts”, J. Pharm. Sci. 66, 1-19 (1977). Non-limiting examples of such salts include:

(1) acid addition salts, formed on a basic or positively charged functionality, by the addition of inorganic acids such as hydrochloric acid, hydrobromic acid, hydroiodic acid, sulfuric acid, sulfamic acid, nitric acid, phosphoric acid, carbonate forming agents, and the like; or formed with organic acids such as acetic acid, propionic acid, lactic acid, oxalic, glycolic acid, pivalic acid, t-butylacetic acid, β-hydroxybutyric acid, valeric acid, hexanoic acid, cyclopentanepropionic acid, pyruvic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, cyclohexylaminosulfonic acid, benzenesulfonic acid, sulfanilic acid, 4-chlorobenzenesulfonic acid, 2-napthalenesulfonic acid, 4-toluenesulfonic acid, camphorsulfonic acid, 3-phenyl propionic acid, lauryl sulphonic acid, lauryl sulfuric acid, oleic acid, palmitic acid, stearic acid, lauric acid, embonic (pamoic) acid, palmoic acid, pantothenic acid, lactobionic acid, alginic acid, galactaric acid, galacturonic acid, gluconic acid, glucoheptonic acid, glutamic acid, naphthoic acid, hydroxynapthoic acid, salicylic acid, ascorbic acid, stearic acid, muconic acid, and the like;

(2) base addition salts, formed when an acidic proton present in the starting compound either is replaced by a metal ion, including, an alkali metal ion (e.g., lithium, sodium, potassium), an alkaline earth ion (e.g., magnesium, calcium, barium), or other metal ions such as aluminum, zinc, iron and the like; or coordinates with an organic base such as ammonia, ethylamine, diethylamine, ethylenediamine, N,N′-dibenzylethylenediamine, ethanolamine, diethanolamine, triethanolamine, tromethamine, N-methylglucamine, piperazine, chloroprocain, procain, choline, lysine and the like.

Pharmaceutically acceptable salts may be synthesized from a starting compound that contains a basic or acidic moiety, by conventional chemical methods. Generally, such salts are prepared by reacting the free acid or base forms of compounds with a stoichiometric amount of the appropriate base or acid in water or in an organic solvent, or in a mixture of the two. Salts may be prepared in situ, during the final isolation or purification of a compound or by separately reacting a compound in its free acid or base form with the desired corresponding base or acid, and isolating the salt thus formed. The term “pharmaceutically acceptable salts” also include zwitterionic compounds containing a cationic group covalently bonded to an anionic group, as they are “internal salts”. It should be understood that all acid, salt, base, and other ionic and non-ionic forms of compounds described herein are intended to be encompassed. For example, if a compound is shown as an acid herein, the salt forms of the compound are also encompassed. Likewise, if a compound is shown as a salt, the acid and/or basic forms are also encompassed.

As used herein, the term “bivalent compound” refers to an organic compound or molecule with two active functional groups in the molecule for connecting (also known as conjugating, coupling, or covalently linking) two other compounds or molecules, to form anew compound or conjugate. For example, 1,12-dodecanedioic is a typical bivalent compound. It can use two carboxyl groups at the 1-position and the 12-position to connect or couple two other molecules with an amide or ester respectively, so as to form a new compound, namely a conjugate of the disclosure. The two active functional groups in bivalent compounds provided herein can be different or the same. Examples of bivalent compounds are given, for example and without limitation, in Formula (I).

As used herein, the term “conjugate” refers to new compounds formed by covalently linking two or more compounds indirectly via intermediary bivalent or multivalent compounds. In accordance with the present disclosure, in some embodiments, conjugates are produced by connecting a bioactive agent (e.g., an siRNA molecule) with a targeting moiety (e.g., a ligand for an extracellular receptor, e.g., a carbohydrate) via an intermediary bivalent compound. Examples of conjugates are given, for example and without limitation, in Formula (V). Such conjugates can, for example, deliver bioactive agents to cell, organ, or tissue targeted by the targeting moiety. In conjugates of the disclosure, the bioactive agent may be connected to the bivalent compound directly or indirectly, e.g., via a carrier group, a linker, etc.

The terms “coupling”, “connecting”, and “conjugating” are used interchangeably herein to refer to the chemical process in which two or more compounds or molecules form new chemical bonds and are covalently linked together via a certain chemical reaction.

The term “amino acid” generally refers to an organic compound comprising both a carboxylic acid group and an amine group. The term “amino acid” includes both “natural” and “unnatural” or “non-natural” amino acids. Additionally, the term amino acid includes O-alkylated or N-alkylated amino acids, as well as amino acids having nitrogen or oxygen-containing side chains (such as Lys, Cys, or Ser) in which the nitrogen or oxygen atom has been acylated or alkylated. Amino acids may be pure L or D isomers or mixtures of L and D isomers, including (but not limited to) racemic mixtures.

The term “natural amino acid” and equivalent expressions refer to L-amino acids commonly found in naturally-occurring proteins. Examples of natural amino acids include, without limitation, alanine (Ala), cysteine (Cys), aspartic acid (Asp), glutamic acid (Glu), phenylalanine (Phe), glycine (Gly), histidine (His), isoleucine (Ile), lysine (Lys), leucine (Leu), methionine (Met), asparagine (Asn), proline (Pro), glutamine (Gln), arginine (Arg), serine (Ser), threonine (Thr), valine (Val), tryptophan (Trp), tyrosine (Tyr), β-alanine (β-Ala), and γ-aminobutyric acid (GABA), etc.

The term “unnatural amino acid” refers to any derivative of a natural amino acid including both D forms, and α- and β-amino acid derivatives. The terms “unnatural amino acid” and “non-natural amino acid” are used interchangeably herein. It is noted that certain amino acids, e.g., hydroxyproline, that are classified as anon-natural amino acid herein, may be found in nature within a certain organism or a particular protein. Amino acids with many different protecting groups appropriate for immediate use in the solid phase synthesis of peptides are commercially available. In addition to the twenty most common naturally occurring amino acids, the following examples of non-natural amino acids and amino acid derivatives may be used according to the invention (common abbreviations in parentheses): 2-aminoadipic acid (Aad), 3-aminoadipic acid (β-Aad), 2-aminobutyric acid (2-Abu), α,β-dehydro-2-aminobutyric acid (8-AU), 1-aminocyclopropane-1-carboxylic acid (ACPC), aminoisobutyric acid (Aib), 3-aminoisobutyric acid ((3-Aib), 2-amino-thiazoline-4-carboxylic acid, 5-aminovaleric acid (5-Ava), 6-aminohexanoic acid (6-Ahx), 2-aminoheptanoic acid (Ahe), 8-aminooctanoic acid (8-Aoc), 11-aminoundecanoic acid (11-Aun), 12-aminododecanoic acid (12-Ado), 2-aminobenzoic acid (2-Abz), 3-aminobenzoic acid (3-Abz), 4-aminobenzoic acid (4-Abz), 4-amino-3-hydroxy-6-methylheptanoic acid (Statine, Sta), aminooxyacetic acid (Aoa), 2-aminotetraline-2-carboxylic acid (ATC), 4-amino-5-cyclohexyl-3-hydroxypentanoic acid (ACHPA), para-aminophenylalanine (4—NH2-Phe), 2-aminopimelic acid (Apm), biphenylalanine (Bip), para-bromophenylalanine (4-Br-Phe), ortho-chlorophenylalanine (2-C1-Phe), meta-chlorophenylalanine (3-C1-Phe), para-chlorophenylalanine (4-C1-Phe), meta-chlorotyrosine (3-C1-Tyr), para-benzoylphenylalanine (Bpa), tert-butylglycine (TLG), cyclohexylalanine (Cha), cyclohexylglycine (Chg), desmosine (Des), 2,2-diaminopimelic acid (Dpm), 2,3-diaminopropionic acid (Dpr), 2,4-diaminobutyric acid (Dbu), 3,4-dichlorophenylalanine (3,4-C1-2-Phe), 3,4-difluororphenylalanine (3,4-F2-Phe), 3,5-diiodotyrosine (3,5-I2-Tyr), N-ethylglycine (EtGly), N-ethylasparagine (EtAsn), ortho-fluorophenylalanine (2-F-Phe), meta-fluorophenylalanine (3-F-Phe), para-fluorophenylalanine (4-F-Phe), meta-fluorotyrosine (3-F-Tyr), homoserine (Hse), homophenylalanine (Hfe), homotyrosine (Htyr), hydroxylysine (Hyl), allo-hydroxylysine (aHyl), 5-hydroxytryptophan (5-OH-Trp), 3- or 4-hydroxyproline (3- or 4-Hyp), para-iodophenylalanine (4-I-Phe), 3-iodotyrosine (3-I-Tyr), indoline-2-carboxylic acid (Idc), isodesmosine (Ide), allo-isoleucine (a-Ile), isonipecotic acid (Inp), N-methylisoleucine (MeIle), N-methyllysine (MeLys), meta-methyltyrosine (3-Me-Tyr), N-methylvaline (MeVal), 1-naphthylalanine (1-Nal), 2-naphthylalanine (2-Nal), para-nitrophenylalanine (4-NO2-Phe), 3-nitrotyrosine (3-NO2-Tyr), norleucine (Nle), norvaline (Nva), ornithine (Orn), ortho-phosphotyrosine (H2P03-Tyr), octahydroindole-2-carboxylic acid (Oic), penicillamine (Pen), pentafluorophenylalanine (F5-Phe), phenylglycine (Phg), pipecolic acid (Pip), propargylglycine (Pra), pyroglutamic acid (PGLU), sarcosine (Sar), tetrahydroisoquinoline-3-carboxylic acid (Tic), thienylalanine, and thiazolidine-4-carboxylic acid (thioproline, Th).

The term “side chain of amino acid” as used herein refers to the side chain of a natural amino acid and/or a non-natural amino acid.

The term “amino acid residue” used herein refers to an incomplete amino acid, that is, the structural fragment remaining after losing at least part of the amino acid molecule. For example, a polypeptide is formed by connecting multiple amino acids with each other through peptide bonds; amino acids in the polypeptide chain are called amino acid residues because some of their groups participate in the formation of peptide bonds. Amino acid residues are not limited to peptide molecules. When amino acids join with other molecules, the incomplete amino acids are collectively referred to as amino acid residues. Similarly, the term “oligopeptide residue” refers to an incomplete oligopeptide.

As used herein, the term “carbohydrate” refers to monosaccharides, disaccharides, trisaccharides and/or polysaccharides. The term “monosaccharide” includes, for example and without limitation, the group of allose, maltose, arabinose, cladose, brown sugar, erythritose, fructose, D-fucitol, L-fucitol, fucosamine, fucose, galactosamine, D-galactosamine, N-acetyl-galactosamine, galactose, glucosamine, N-acetyl-glucosamine, glucosamine, glucose, glucose-6-phosphate, glucose glyceraldehyde, L-glycerin-D-mannose-heptose, glycerol, glycerol, glucose, iodose, lysose, mannosamine, mannose, mannose-6-phosphate, psicose, quinovose, quinovosamine, rhamnol, rhamnose, ribose, ketulose, heptose, sorbose, tagatose, talose, tartaric acid, threose, xylose or xylose. A monosaccharide can be of D- or L-configuration. A monosaccharide can also be a deoxysaccharide (a hydroxyl alcohol substituted by hydrogen), an amino saccharide (a hydroxyl alcohol substituted by amino), a thiosaccharide (a hydroxyl alcohol substituted by mercaptan, or a carbon monoxide substituted by CS, or cyclic epoxide substituted by sulfur), a selenoglycan, a tellurite, an azasaccharide (a cyclic carbon substituted by nitrogen), an imino saccharide (an epoxy substituted by nitrogen), a phosphosaccharide (an epoxide substituted by phosphorus), a phosphosaccharide (a cyclic carbon substituted by phosphorus), a c-substituted monosaccharide (a hydrogen on a non-terminal carbon atom substituted by carbon), an unsaturated monosaccharide, a sugar alcohol (a carbonyl group substituted by a CHOH group), an aldose acid (an aldehyde group substituted by carboxyl group), a ketoalduronic acid, a glucuronic acid, an aldose acid, and the like. Amino sugars include, for example and without limitation, aminomonosaccharide, such as galactosamine, glucosamine, mannosamine, fucosamine, quinvolosamine, neuraminic acid, cell wall amide acid, lactosamine, arcosamine, Bacillus glucosamine, daunorosamine, desosamine, fluxamine, carbamate, mannosamine, trehalose, myramine, peroxidase amine, pneumonamine, puronamine, or Rhodamine. Monosaccharides and the like can be further replaced and substituted.

The terms “disaccharide”, “trisaccharide” and “polysaccharide” include, for example and without limitation, groups such as abalone sugar, aclaboose, glucosamine, amylopectin, apigenin, glucosamine, ascorbic acid, flavonoid sugar, cellobiose, cellotriose, cellulose, chacotriose, thioether, chitin, collagen, cyclodextrin, melamine, dextrin, 2-deoxyribose, 2-deoxyglucose, diglucose, maltose, digital ketose, evalose, evodia rutaecarpa, fructooligosaccharide, galactooligosaccharide, gentian, gentian disaccharide, dextran, glycogen, glycogen, hamameliose, heparin, inulin, isoevodia saponin, isomaltose, isomaltotriose, isopentose, koji polysaccharide, lactose, lactosamine, lactodiamine, layered arabinose, L-glucan, L-glucanone, maltose, mannooligosaccharide, mannotriose, maltose, melibiose, celluar amidic acid, trehalose, neuraminic acid, glucosamine, norgindamycin, Sophorae, stachyose, Streptococcus, sucrose, or trehalose. Disaccharides, trisaccharides and polysaccharides can be further replaced and substituted. Disaccharides can also include amino sugars and their derivatives, for example and without limitation, mycosaminoglycans derived at the C-4′ position or 4-deoxy-3-amino-glucose derived at the C-6′ position.

It should also be understood that various hydroxyl protecting groups may be used in compounds of the disclosure or during synthesis thereof. In general, a protective group makes a chemical functional group insensitive to specific reaction conditions and can be added or removed on the functional group in a molecule without substantially damaging the rest of the molecule. Representative hydroxyl protecting groups are disclosed in Beausage et al., Tetrahedron 1992,48,2223-2311, and Green and Wuts, Protective Groups in Organic Synthesis, Chapter 2,2d ed, John Wiley & Sons, New York, 1991, which is incorporated by reference herein in its entirety. In some embodiments, the protective group is stable under alkaline conditions, but can be removed under acidic conditions. In some embodiments, non-limiting examples of hydroxyl protecting groups that can be used herein include dimethoxytriphenylmethyl (DMT), monomethoxytriphenylmethyl, 9-phenyloxaanthracene-9-yl(Pixyl) and 9-(p-methoxyphenyl) oxaanthracene-9-yl(Mox). In some embodiments, non-limiting examples of hydroxyl protecting groups that can be used herein include triphenylmethyl(Tr), 4-methoxytrimethylphenyl(MMTr), 4,4′-dimethoxytriphenylmethyl(DMTr) and 4,4′,4′-trimethoxytriphenylmethyl(TMTr).

### Bioactive Agents

As used herein, the term “bioactive agent” refers to a therapeutic compound useful for treating or preventing a disease or disorder in a subject. The bioactive agent is not meant to be particularly limited and may be any therapeutic compound for which targeting to a certain cell or tissue is beneficial. Any beneficial therapeutic or prophylactic compound suitable for conjugation, as described herein, may be used in conjugates of the disclosure. Non-limiting examples of bioactive agents include antibodies, oligonucleotides, hormones, antibiotics, low molecular weight compounds, drugs, prodrugs, and combinations thereof. In one embodiment, the bioactive agent is an oligonucleotide such as a ribonucleic acid (RNA) or an RNAi agent or a derivative thereof. In some embodiments, the bioactive agent is an oligonucleotide such as a single stranded or double stranded RNA or a derivative thereof. For example and without limitation, the bioactive agent may be an RNA therapeutic such as an RNAi agent, a small interfering RNA (siRNA), an siRNA-drug and/or -prodrug conjugate, an RNA aptamer, or an antisense RNA.

In some embodiments, the bioactive agent is a therapeutic compound for which delivery to the liver is beneficial for treatment or prevention of a disease or disorder in a subject.

In some embodiments, “bioactive agent” refers to a complete active molecule; in other embodiments, “bioactive agent” refers to the bioactive part, group or moiety in a conjugate of the disclosure. It should be understood that a bioactive agent can be a bioactive molecule or the bioactive part/group/moiety of a conjugate, depending on the context.

In some embodiments, a bioactive agent comprises an active functional group suitable for covalent coupling to one end of the bivalent compound of the disclosure. A bioactive agent may be connected directly to the bivalent compound or indirectly, e.g., through a linker or through a carrier group, e.g., —L—P(O)(OH)—, wherein L is a linker, In some embodiments, bioactive agents are directly coupled with bivalent compounds of the disclosure. In other embodiments, a bioactive agent is first connected with a carrier group such as —L— P(O)(OH)—, and then coupled with the bivalent compound through the carrier group to form a conjugate, as shown in Formula (V).

The terms “iRNA”, “RNAi agent,” “iRNA agent,”, and “RNA interference agent” are used interchangeably herein to refer to a bioactive agent that contains RNA and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a gene in a cell, e.g., a cell within a subject, such as a mammalian subject, such as a human.

In one embodiment, an RNAi agent of the disclosure includes a single stranded RNA that interacts with a target RNA sequence, e.g., a PCSK9 target mRNA sequence, to direct the cleavage of the target RNA. Without wishing to be bound by theory, it is believed that long double stranded RNA introduced into cells is broken down into siRNA by a Type III endonuclease known as Dicer (Sharp et al. (2001) Genes Dev. 15:485). Dicer, a ribonuclease III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in some embodiments the bioactive agent comprises an oligonucleotide which generates a single stranded RNA (siRNA) within a cell and which promotes the formation of a RISC complex to effect silencing of the target gene, such as a PCSK9 gene. Accordingly, the term “siRNA” is also used herein to refer to an RNAi as described above.

In another embodiment, an RNAi agent may be a single-stranded siRNA that is introduced into a cell or organism to inhibit a target mRNA. Single-stranded RNAi agents can bind to the RISC endonuclease Argonaute 2, which then cleaves the target mRNA. The single stranded siRNAs are generally 15-30 nucleotides in length and may be chemically modified. The design and testing of single-stranded siRNAs are described in U.S. Pat. No. 8,101,348 and in Lima et al., (2012) Cell 150: 883-894, the entire contents of each of which are hereby incorporated herein by reference. Any of the antisense nucleotide sequences described herein may be used as a single-stranded siRNA as described herein or as chemically modified by the methods described e.g. in Lima et al., (2012) Cell 150:883-894.

In some embodiments, an “siRNA” for use in the compounds, compositions, and methods of the disclosure is a double-stranded RNA and is referred to herein as a “double stranded RNAi agent,” “double-stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, such as a PCSK9 gene. In some embodiments, a double-stranded RNA (dsRNA) triggers the degradation of a target RNA, e.g., an mRNA, through a post-transcriptional gene-silencing mechanism referred to herein as RNA interference or RNAi.

In some embodiments, RNAi agents of the disclosure are defined by reference to SEQ ID NO. Double stranded RNAi agents comprising a sense strand and an antisense strand may be referenced by two SEQ ID NOs. divided by a “/”, where the first number defines the sense strand and the second number defines the antisense strand (unless clearly indicated otherwise). For example, as used herein, the “sequence set forth in SEQ ID NO: 1/2” refers to a double stranded agent comprising both SEQ ID NOs: 1 and 2, e.g., wherein the sense strand has or comprises the sequence set forth in SEQ ID NO: 1 and the antisense strand has or comprises the sequence set forth in SEQ ID NO: 2.

In general, the majority of nucleotides of each strand of a dsRNA molecule are ribonucleotides, but as described in detail herein, each or both strands can also include one or more non-ribonucleotides, e.g., a deoxyribonucleotide and/or a modified nucleotide. In addition, an oligonucleotide of the disclosure, e.g, an “RNAi agent” or an “siRNA”, may include one or more ribonucleotides with chemical modifications. Such modifications may include all types of modifications described herein or known in the art. Any such modifications are encompassed by “RNAi agent” and by “siRNA” herein.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” Where the two strands are connected covalently by means other than an uninterrupted chain of nucleotides between the 3’-end of one strand and the 5 ′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi agent may comprise one or more nucleotide overhangs.

In one embodiment, an RNAi agent of the invention is a dsRNA of 24-30 nucleotides that interacts with a target RNA sequence, e.g., a gene target mRNA sequence, e.g., a PCSK9 target mRNA sequence, to direct the cleavage of the target RNA. The term “siRNA” is used to refer to an RNAi agent that targets a particular gene target mRNA sequence; for example, “PCSK9-siRNA” and “ANGPTL3-siRNA” refer respectively to RNAi agents that target PCSK9 and ANGPTL3 mRNA sequences. Without wishing to be bound by theory, long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15: 188).

As used herein, a “nucleotide overhang” refers to the unpaired nucleotide or nucleotides that protrude from the duplex structure of an RNAi agent when a 3′-end of one strand of the RNAi agent extends beyond the 5′-end of the other strand, or vice versa. “Blunt” or “blunt end” means that there are no unpaired nucleotides at that end of the double stranded RNAi agent, i.e., no nucleotide overhang. A “blunt ended” RNAi agent is a dsRNA that is double-stranded over its entire length, i.e., no nucleotide overhang at either end of the molecule. In some embodiments, the RNAi agents of the invention include RNAi agents with nucleotide overhangs at one end (i.e., agents with one overhang and one blunt end) or with nucleotide overhangs at both ends.

The term “antisense strand” refers to the strand of a double stranded RNAi agent which includes a region that is substantially complementary to a target sequence (e.g., a human PCSK9 mRNA). As used herein, the term “region complementary to part of an mRNA encoding PCSK9” refers to a region on the antisense strand that is substantially complementary to part of a PCSK9 mRNA sequence. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand. As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. For example, a complementary sequence is sufficient to allow the relevant function of the nucleic acid to proceed, e.g., RNAi. The skilled person will be able to determine the set of conditions most appropriate for a test of complementarity of two sequences in accordance with the ultimate application of the hybridized nucleotides.

In some embodiments, a bioactive agent is a functional oligonucleotide, such as for example and without limitation, an RNAi agent, e.g., a small interfering RNA (siRNA), microRNA, anti-microRNA, microRNA antagonist, microRNA mimic, decoy oligonucleotide, immunostimulator, guanine (G) quadruplex DNA or RNA, guanine (G) tetraplex DNA or RNA, alternative splice, single stranded RNA, double stranded RNA, antisense nucleic acid, aptamer, stem loop RNA, mRNA fragment, and active RNA or DNA.

In some embodiments, the functional oligonucleotide is a single stranded oligonucleotide; and the carrier group is connected to the end of the single stranded oligonucleotide, where the end of the single stranded oligonucleotide refers to the first four nucleotides from one end of the single stranded oligonucleotide. In other embodiments, the functional oligonucleotide is a single stranded oligonucleotide; and the carrier group is connected at the end of the single stranded oligonucleotide. In some embodiments, the carrier group is connected to the 3′ or the 5′ end of the single stranded oligonucleotide. In some embodiments, the bivalent compound of the disclosure is attached directly to the 3′ or the 5′ end of the single stranded oligonucleotide.

In some embodiments, the functional oligonucleotide is a double stranded oligonucleotide comprising a sense chain and an antisense chain, and the carrier group is attached to one end of the double stranded oligonucleotide. In some embodiments, double stranded oligonucleotides are siRNAs, each strand of which may comprise independently modified or unmodified nucleotides.

It should be understood that any nucleotide or nucleoside in a functional oligonucleotide may be independently modified or unmodified. For example, it is well known to those skilled in the art that modified nucleotide group(s) can be introduced into an oligonucleotide such as an siRNA by using a nucleoside monomer with the desired modification. Methods for preparing such nucleoside monomers with desired modification(s) and the method for introducing modified nucleotide group(s) into an oligonucleotide such as an siRNA are also well known to those skilled in the art. Many modified nucleoside monomers are commercially available or can be prepared by known methods.

In some embodiments, a functional oligonucleotide comprises at least one phosphorothioate or methylphosphonate internucleotide linkage. In some such embodiments, the phosphorothioate or methylphosphonate internucleotide linkage is at the 3′-terminus of one strand. In one such embodiment, the strand is the antisense strand; in another embodiment, the strand is the sense strand. In alternative embodiments, the phosphorothioate or methylphosphonate internucleotide linkage is at the 5′-terminus of one strand. In an embodiment, the strand is the antisense strand; in another embodiment, the strand is the sense strand. In yet other embodiments, the phosphorothioate or methylphosphonate internucleotide linkage is at both the 5′- and 3′-terminus of one strand. In one embodiment, the strand is the antisense strand; in another embodiment, the strand is the sense strand.

In an embodiment, modifications on nucleotides are selected from the group consisting of LNA, HNA, CeNA, 2′-methoxyethyl, 2′-O-alkyl (e.g., 2′-O-methyl), 2′-O-allyl, 2′-C- allyl, 2′-fluoro, 2′-deoxy, 2′-hydroxyl, and combinations thereof. In another embodiment, modifications on the nucleotides are 2′-O-methyl or 2′-fluoro modifications.

In an embodiment, the base pair at the 1 position of the 5′-end of the antisense strand of the duplex in an siRNA is an AU base pair.

In some embodiments of conjugates of the disclosure, the bioactive agent is a functional oligonucleotide, e.g., an siRNA, e.g., a double-stranded siRNA, wherein the bioactive agent is capable of inhibiting the expression of a gene selected from ApoB, ApoC, ANGPTL3, PCSK9, SCD1, FVII, p53, HBV, and HCV. In some such embodiments, the siRNA comprises a sense strand complementary to an antisense strand, wherein the antisense strand comprises a region complementary to part of an mRNA encoding the selected gene. In some such embodiments, each strand is about 14 to about 30 nucleotides in length. In some such embodiments, each strand independently comprises one or more modified nucleotide, e.g., a modification selected from LNA, HNA, CeNA, 2′-methoxyethyl, 2′-O-alkyl (e.g., 2′-O-methyl), 2′-O-allyl, 2′-C- allyl, 2′-fluoro, 2′-deoxy, 2′-hydroxyl, and combinations thereof.

In some embodiments, each strand of an RNAi agent, e.g., an siRNA, is about 12-30 nucleotides in length. For example, each strand may be between 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

For a double stranded RNA, the duplex region may be 12-30 nucleotides in length. For example, the duplex region can be between 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In some embodiments, an RNAi agent, e.g., an siRNA, may contain one or more overhang regions and/or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-Omethyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

In one embodiment, the 5′- or 3 ′-overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3’-end of the sense strand, antisense strand, or both strands. In one embodiment, this 3′-overhang is present in the antisense strand. In one embodiment, this 3′-overhang is present in the sense strand.

The RNAi agent, e.g., siRNA, may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RN Ai has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent results in an siRNA comprising the 3′ end of the second strand, thereby reducing expression of the target gene in the mammal.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

In some embodiments, every nucleotide in the sense strand and antisense strand of the RNAi agent, including the nucleotides that are part of the motifs, may be independently modified. Each nucleotide may be modified with the same or different modification which can include one or more alteration of one or both of the non-linking phosphate oxygens and/or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone. In an embodiment, each strand comprises at least 1-5 modified nucleotides, 5-10 modified nucleotides, or more than 5 modified nucleotides.

In some embodiments of conjugates of the disclosure, the bioactive agent is a therapeutic siRNA selected from the siRNAs listed in Table 2 and Table 3.

In some embodiments, a bioactive agent comprises a functional oligonucleotide which is an siRNA specific for a gene selected from ApoB, ApoC, ANGPTL3, PCSK9, SCD1, FV II, p53, HBV and HCV. In some such embodiments, the conjugate comprising the bioactive agent is used to treat a subject having a condition mediated by or associated with ApoB, ApoC, ANGPTL3, PCSK9, SCD1, FV II, p53, HBV or HCV expression respectively.

In some embodiments, a bioactive agent comprises a functional oligonucleotide which is a double stranded siRNA for which the target gene is PCSK9. In some such embodiments, the conjugate comprising the bioactive agent is used to treat a subject having a condition mediated by or associated with PCSK9 expression.

In some embodiments, a bioactive agent comprises a functional oligonucleotide which is a double stranded siRNA for which the target gene is ANGPTL3. In some such embodiments, the conjugate comprising the bioactive agent is used to treat a subject having a condition mediated by or associated with ANGPTL3 expression.

In some embodiments, a bioactive agent comprises a functional oligonucleotide which is a double stranded siRNA for which the target gene is ApoB. In some such embodiments, the conjugate comprising the bioactive agent is used to treat a subject having a condition mediated by or associated with ApoB expression.

In some embodiments, a bioactive agent comprises a functional oligonucleotide which is a double stranded siRNA for which the target gene is FVII. In some such embodiments, the conjugate comprising the bioactive agent is used to treat a subject having a condition mediated by or associated with FVII expression.

### Carrier Groups

In conjugates of the disclosure, bioactive agents and bivalent compounds can be connected in many ways, including but not limited to direct or indirect coupling. Indirect coupling can be accomplished using a wide variety of carrier groups. In some embodiments of conjugates of the disclosure, a phosphonyl group in the carrier group is used for connecting the bioactive agent. However, it should be understood that the carrier group is not particularly limited. Any suitable carrier group comprising an active functional group may be used to connect a bioactive agent to a bivalent compound in accordance with the disclosure. Further, the active functional group is not particularly limited, and there are many different active functional groups that may be included on a bioactive agent and/or a carrier group.

Generally, a carrier group is coupled with a bioactive agent and/or a bivalent compound of the disclosure through the active functional groups. In some embodiments, a carrier group is a ring structure. In some such embodiments, the ring structure is a carbon ring system, that is, wherein all the ring atoms are carbon atoms, or a heterocyclic ring system, that is, wherein one or more of the ring atoms are heteroatoms, such as nitrogen, oxygen, or sulfur. The ring structure may be a single ring system or may contain two or more rings, such as fused rings. The ring structure may be a fully saturated ring system, or it may contain one or more double bonds.

In some embodiments, the carrier group includes a nitrogen-containing heterocyclic ring, for example a 4-membered ring, a 5-membered ring, or a six-membered ring. In some embodiments, the nitrogen-containing heterocyclic ring has at least one substituent containing an active functional group, in which case the nitrogen-containing heterocyclic ring is coupled with the bioactive agent through the active functional group on the substituent, and is coupled with one end of the bivalent compound of the disclosure through the imino group on the ring, such that the bioactive agent and the bivalent compound are indirectly coupled via the carrier group.

### Targeting Moieties

As used herein, the term “targeting moiety” refers to an organic moiety comprising one or more carbohydrate, one or more polypeptide, and/or one or more lipophile component, which is capable of targeting the conjugate of the disclosure to a desired cell, organ or tissue. For example and without limitation, in some embodiments the targeting moiety binds to a cell-surface receptor on a target cell. The conjugate may then be internalized into the target cell, e.g., via receptor-mediated endocytosis, and released inside the cell. In this way the targeting moiety can facilitate delivery of a bioactive agent to a target cell, organ, or tissue, where it can act to treat and/or prevent a disease or disorder. In some embodiments, the targeting moiety is capable of targeting the conjugate to hepatic cells and tissues.

In an embodiment, a targeting moiety is a ligand for a cell-surface receptor on a target cell or tissue. Thus in some embodiments, the targeting moiety binds to a cell-surface receptor. In some embodiments, a targeting moiety is a biological ligand, e.g., a liver-targeting biological ligand. It should be understood that the targeting moiety is not particularly limited and that any suitable targeting moiety for delivering a bioactive agent to a desired cell, organ or tissue may be used. Any cell-surface receptor or biomarker or part thereof which constitutes a suitable target may be targeted by the targeting moiety.

In some embodiments, the targeting moiety specifically binds to a specific receptor on cells in a specific tissue to achieve tissue-specific targeting. The conjugates of the disclosure thus have potential application for modulating or silencing expression of a wide range of genes in a wide range of cells, organs and tissues.

In some embodiments, the targeting moiety specifically binds to a receptor expressed on the surface of hepatocytes, thereby specifically targeting the liver/liver tissue. In some embodiments, the targeting moiety specifically targets a hepatocyte-specific cell-surface receptor. In some embodiments, the targeting moiety comprises or consists of N-acetyl galactosamine (GalNAc) and specifically binds to asialoglycoprotein receptors (ASGPRs) on the surface of hepatocytes. In one such embodiment, the targeting moiety comprises or consists of triantennary GalNAc or a derivative thereof.

In some such embodiments, the conjugate of the disclosure has excellent liver targeting specificity, so that it is able to effectively deliver a connected functional oligonucleotide (e.g., siRNA) directly to the liver, thereby modulating (e.g., inhibiting) specific gene expression in liver cells. In some embodiments, endogenous genes expressed in the liver are specifically modulated, e.g., inhibited and/or silenced.

### Compositions

In an embodiment, there is provided a pharmaceutical composition comprising a compound of the disclosure, e.g., a compound of Formula (I), (II), (III), (IV), or (V), a compound of Table 1, or a pharmaceutically acceptable salt or ester thereof, and a pharmaceutically acceptable carrier.

The term “Pharmaceutical composition” as used herein refers to a composition comprising a compound of the disclosure and at least one component comprising a pharmaceutically acceptable carrier, diluent, adjuvant, excipient, or vehicle, such as a preserving agent, a filler, a disintegrating agent, a wetting agent, an emulsifying agent, a suspending agent, a sweetening agent, a flavoring agent, a perfuming agent, an antibacterial agent, an antifungal agent, a lubricating agent, a dispensing agent, and the like, depending on the nature of the mode of administration and dosage forms.

“Pharmaceutically acceptable carrier” refers to a diluent, adjuvant, excipient, vehicle or carrier with which a compound is administered. The terms “Pharmaceutically acceptable vehicle” and “Pharmaceutically acceptable carrier” are used interchangeably herein.

The preparation of pharmaceutical compositions can be carried out as known in the art (see, for example, Remington: The Science and Practice of Pharmacy, 20th Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

Any suitable pharmaceutically acceptable carriers, diluents, adjuvants, excipients, or vehicles may be used in compositions provided herein, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

A pharmaceutically acceptable carrier may include any and all solvents, dispersion media, coatings, antibacterial and antifungal agents, isotonic and absorption delaying agents, and the like that are physiologically compatible. In one embodiment, the carrier is suitable for oral administration. Alternatively, the carrier may be suitable for intravenous, intraperitoneal, intramuscular, sublingual or parenteral administration. In other embodiments, the carrier is suitable for topical administration or for administration via inhalation. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated.

A pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Many methods for the preparation of such formulations are generally known to those skilled in the art. Sterile injectable solutions can be prepared by incorporating an active compound, such as a compound of Formula I, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the disclosure may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a disease of interest, e.g., a PCSK9-related disease or disorder, such as hypercholesterolemia, dyslipidemia, ASCVD, type 2 diabetes, etc.. Dosages are discussed further below.

In some embodiments, there are provided pharmaceutical compositions that comprise a therapeutically effective amount of a compound of the disclosure, and a pharmaceutically acceptable carrier. In an embodiment, there are provided pharmaceutical compositions for the treatment or prevention of a PCSK9-related disease or disorder, comprising a compound of the disclosure, or a pharmaceutically acceptable salt or ester thereof, and a pharmaceutically acceptable carrier. In some such embodiments, the compound is a compound set forth in Table 1, or a pharmaceutically acceptable salt or ester thereof. In some such embodiments, the compound is compound 5.

Supplementary active compounds can also be incorporated into the compositions provided herein. For example, a pharmaceutical composition provided herein may further comprise at least one additional therapeutic agent, as discussed below. In an embodiment, there are provided pharmaceutical compositions comprising at least one compound of the disclosure, together with a pharmaceutically acceptable carrier suitable for administration to a human or animal subject, either alone or together with one or more other therapeutic agent.

Consequently, there is provided a combined pharmaceutical composition, e.g. for use in any of the methods described herein, comprising a compound of the disclosure in free form or pharmaceutically acceptable salt form in association with a pharmaceutically acceptable carrier. A combined pharmaceutical composition may comprise a compound of the disclosure in free form or in pharmaceutically acceptable salt form as active ingredient; one or more pharmaceutically acceptable carrier material(s); and optionally one or more further drug substances. Such combined pharmaceutical composition may be in the form of one dosage unit form or as a kit of parts. a combined pharmaceutical composition comprising a therapeutically effective amount of a compound of the disclosure in free form or in pharmaceutically acceptable salt form and a second drug substance, for simultaneous or sequential administration.

“Combination” refers to either a fixed combination in one dosage unit form, or a kit of parts for the combined administration where a compound of the disclosure and a combination partner (i.e., another therapeutic agent or drug as described further below), may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the disclosure and a combination partner, are both administered to a patient simultaneously in the form of a single entity or dosage. The term “non-fixed combination” means that the active ingredients, e.g. a compound of the disclosure and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The amount of the compound of the disclosure in a formulation can vary within the full range employed by those skilled in the art. Typically, a formulation will contain, on a weight percent (wt %) basis, from about 0.01-99.99 wt % of a compound of the disclosure based on the total formulation, with the balance being one or more suitable pharmaceutical excipients. In some embodiments, the compound is present at a level of about 1-80 wt %. Dosages are discussed further below.

In one embodiment, there is provided a pharmaceutical composition comprising a conjugate of the disclosure, or a pharmaceutically acceptable salt or ester thereof, and a pharmaceutically acceptable carrier, wherein the bioactive agent is a PCSK9-siRNA. Use of such pharmaceutical composition for the treatment or prevention of a PCSK9-related disease or disorder is also provided.

The present disclosure also provides a kit including one or more compound of the disclosure and optionally a combination partner as disclosed herein. In one embodiment, a kit comprises a compound or composition of the disclosure and a package insert or other labeling including directions for use thereof. Kits may optionally include one or more additional component such as acids, bases, buffering agents, inorganic salts, solvents, antioxidants, preservatives, or metal chelators; devices for delivery and administration such as syringes, vials, and the like; and instructions for use thereof. In one embodiment, a kit further comprises means for contacting a cell with a compound or composition of the disclosure (e.g., comprising a bioactive agent which is an RNAi agent, e.g., an siRNA), e.g., an injection device, or means for measuring the inhibition of PCSK9 in a subject, e.g., means for measuring the inhibition of PCSK9 mRNA. Such means for measuring the inhibition of PCSK9 may comprise a means for obtaining a sample from a subject, such as, e.g., a plasma sample. Kits may optionally further comprise means for administering the compound or composition to a subject or means for determining the therapeutically effective or prophylactically effective amount.

### Methods of Use

There are provided methods of treating or preventing disease comprising administration of the compounds and compositions of the disclosure to a subject in need thereof. Without wishing to be limited by theory, it is believed that compounds of the disclosure can efficiently deliver bioactive agents to target cells, organs, and tissues, such as in the liver, and thereby potentiate their therapeutic and/or prophylactic effect.

In some embodiments, there are provided methods for inhibiting the expression of a target gene in a cell, comprising contacting the cell with a compound or composition of the disclosure, and maintaining the cell for enough time to inhibit the expression of the target gene in the cell. In some such embodiments, the degradation of the mRNA transcript of the target gene is obtained, and expression of the target gene is thereby inhibited. In some embodiments, the expression of the target gene is inhibited by at least 30%, at least about 40%, at least about 50%, at least about 60%, at least 70%, or at least 80%.

Methods for inhibiting expression of a target gene are discussed further hereinbelow, using inhibition of PCSK9 as an example. It should be understood that discussion of PCSK9 is exemplary only and is not meant to be limiting. The discussion herein and below is intended to apply mutatis mutandis to inhibiting expression of any desired target gene as well as to treating or preventing diseases or disorders associated with or mediated by expression of the desired target gene. In some embodiments, the discussion herein and below applies mutatis mutandis to inhibiting expression of a gene selected from ApoB, ApoC, ANGPTL3, PCSK9, SCD1, FVII, p53, HBV, and HCV as well as to treating or preventing diseases or disorders associated with or mediated by expression of a gene selected from ApoB, ApoC, ANGPTL3, PCSK9, SCD1, FVII, p53, HBV, and HCV.

In some embodiments, there are provided methods of inhibiting expression of Proprotein Convertase Subtilisin Kexin 9 (PCSK9) in a cell. The methods include contacting a cell with a compound of the disclosure (or a composition thereof) wherein the bioactive agent is an RNAi agent, e.g., a double stranded RNAi agent, e.g., an siRNA, in an amount effective to inhibit expression of PCSK9 in the cell, thereby inhibiting expression of PCSK9 in the cell.

Contacting of a cell with a compound or composition of the disclosure may be done in vitro or in vivo. Contacting a cell in vivo with the compound or composition includes contacting a cell or group of cells within a subject, e.g., a human subject, with the compound or composition. Combinations of in vitro and in vivo methods of contacting are also possible. Contacting may be direct or indirect. Furthermore, contacting a cell may be accomplished via a targeting ligand, including any ligand described herein or known in the art. In some embodiments, the targeting ligand is a carbohydrate moiety, e.g., a triantennary-GalNAc ligand, or any other ligand that directs the bioactive agent (e.g., siRNA) to a site of interest, e.g., the liver of a subject.

The term “inhibiting” as used herein, is used interchangeably with “reducing,” “silencing,” “downregulating,” “suppressing” and other similar terms, and includes any level of inhibition.

The phrase “inhibiting expression of PCSK9” is intended to refer to inhibition of expression of any PCSK9 gene (such as, e.g., a mouse PCSK9 gene, a rat PCSK9 gene, a monkey PCSK9 gene, or a human PCSK9 gene) as well as variants or mutants of a PCSK9 gene. Thus, the PCSK9 gene may be a wild-type PCSK9 gene, a mutant PCSK9 gene, or a transgenic PCSK9 gene in the context of a genetically manipulated cell, group of cells, or organism.

“Inhibiting expression of a PCSK9 gene” includes any level of inhibition of a PCSK9 gene, e.g., at least partial suppression of the expression of a PCSK9 gene. The expression of the PCSK9 gene may be assessed based on the level, or the change in the level, of any variable associated with PCSK9 gene expression, e.g., PCSK9 mRNA level, PCSK9 protein level, or serum lipid levels. This level may be assessed in an individual cell or in a group of cells, including, for example, a sample derived from a subject. Inhibition may be assessed by a decrease in an absolute or relative level of one or more variables that are associated with PCSK9 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of a PCSK9 gene is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%. at least about 95%, at least about 96%, at least about 97%, at least about 98%, or at least about 99%.

Inhibition of the expression of a PCSK9 gene may be manifested by a reduction of the amount of mRNA expressed by a first cell or group of cells (such cells may be present, for example, in a sample derived from a subject) in which a PCSK9 gene is transcribed and which has or have been treated (e.g., by contacting the cell or cells with a compound or composition of the disclosure, or by administering a compound or composition of the disclosure to a subject in which the cells are or were present) such that the expression of a PCSK9 gene is inhibited, as compared to a second cell or group of cells substantially identical to the first cell or group of cells but which has not or have not been so treated (control cell(s)). In some embodiments, the inhibition is assessed by expressing the level of mRNA in treated cells as a percentage of the level of mRNA in control cells.

Alternatively, inhibition of the expression of a PCSK9 gene may be assessed in terms of a reduction of a parameter that is functionally linked to PCSK9 gene expression, e.g., PCSK9 protein expression, such as lipid levels, cholesterol levels, e.g., LDL-C levels. PCSK9 gene silencing may be determined in any cell expressing PCSK9, either constitutively or by genomic engineering, and by any assay known in the art. The liver is the major site of PCSK9 expression. Other significant sites of expression include the pancreas, kidney, and intestines.

Inhibition of the expression of a PCSK9 protein may be manifested by a reduction in the level of the PCSK9 protein that is expressed by a cell or group of cells (e.g., the level of protein expressed in a sample derived from a subject). As explained above for the assessment of mRNA suppression, the inhibition of protein expression levels in a treated cell or group of cells may similarly be expressed as a percentage of the level of protein in a control cell or group of cells. A control cell or group of cells that may be used to assess the inhibition of the expression of a PCSK9 gene includes a cell or group of cells that has not yet been contacted with a compound or composition of the disclosure. For example, the control cell or group of cells may be derived from an individual subject (e.g., a human or animal subject) prior to treatment of the subject with the compound or composition of the disclosure.

The level of PCSK9 mRNA that is expressed by a cell or group of cells may be determined using any method known in the art for assessing mRNA expression. In one embodiment, the level of expression of PCSK9 in a sample is determined by detecting a transcribed polynucleotide, or portion thereof, e.g., mRNA of the PCSK9 gene. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilizing ribonucleic acid hybridization include nuclear run-on assays, RT-PCR, RNase protection assays (Melton et al., Nuc. Acids Res. 12:7035), Northern blotting, in situ hybridization, and microarray analysis.

In one embodiment, the level of expression of PCSK9 is determined using a nucleic acid probe. The term “probe”, as used herein, refers to any molecule that is capable of selectively binding to a specific PCSK9. Probes can be synthesized by one of skill in the art or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or Northern analyses, polymerase chain reaction (PCR), analyses of isolated mRNA with a nucleic acid molecule (probe) that can hybridize to PCSK9 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of PCSK9 mRNA.

An alternative method for determining the level of expression of PCSK9 in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular embodiments, the level of expression of PCSK9 is determined by quantitative fluorogenic RT-PCR (i.e., the TaqMan™ System). The expression levels of PCSK9 mRNA may be monitored using a membrane blot (such as used in hybridization analysis such as Northern, Southern, dot, and the like), or microwells, sample tubes, gels, beads or fibers (or any solid support comprising bound nucleic acids). See, e.g., U.S. Pat. Nos. 5,770,722, 5,874,219, 5,744,305, 5,677,195 and 5,445,934. The determination of PCSK9 expression level may also comprise using nucleic acid probes in solution. In some embodiments, the level of mRNA expression is assessed using branched DNA (bDNA) assays or real time PCR (qPCR).

The level of PCSK9 protein expression may be determined using any method known in the art for the measurement of protein levels. Such methods include, for example and without limitation, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), Immunoelectrophoresis, Western blotting, radioimmunoassay (RIA), enzyme-linked immunosorbent assays (ELISAs), immunofluorescent assays, electrochemiluminescence assays, and the like.

The term “sample” as used herein refers to a collection of similar fluids, cells, or tissues isolated from a subject, as well as fluids, cells, or tissues present within a subject. Examples of biological fluids include blood, serum and serosal fluids, plasma, lymph, urine, cerebrospinal fluid, saliva, ocular fluids, and the like. Tissue samples may include samples from tissues, organs or localized regions. For example, samples may be derived from particular organs, parts of organs, or fluids or cells within those organs. In some embodiments, samples may be derived from the liver (e.g., whole liver or certain segments of liver or certain types of cells in the liver, such as, e.g., hepatocytes). In some embodiments, a “sample derived from a subject” refers to blood or plasma drawn from the subject. In further embodiments, a “sample derived from a subject” refers to liver tissue derived from the subject.

In some embodiments of the methods of the disclosure, the compound or composition is administered to a subject such that the bioactive agent is delivered to a specific site within the subject. The inhibition of expression of a gene may be assessed using measurements of the level or change in the level of the corresponding mRNA or protein in a sample derived from fluid or tissue from the specific site within the subject. For example, the inhibition of expression of PCSK9 may be assessed using measurements of the level or change in the level of PCSK9 mRNA or PCSK9 protein in a sample derived from fluid or tissue from the specific site within the subject. In some embodiments, the site is the liver. The site may also be a subsection or subgroup of cells from any one of the aforementioned sites. The site may also include cells that express a particular type of receptor.

In some embodiments, there are provided methods for treating or preventing a PCSK9-related disease or disorder. The term “PCSK9-related disease or disorder” is intended to include any disease or disorder associated with the PCSK9 gene or protein. Such a disease or disorder may be caused, for example, by excess production of the PCSK9 protein, by PCSK9 gene mutations, by abnormal cleavage of the PCSK9 protein, by abnormal interactions between PCSK9 and other protein or other endogenous or exogenous substances.

In some embodiments, there are provided methods for treating or preventing diseases and disorders that can be modulated by down regulating PCSK9 gene expression. In an embodiment, the invention relates to the treatment of a PCSK9-related disease or disorder, i.e., a disease or disorder ameliorated by inhibition of PCSK9, especially such disorders that respond in a beneficial way to the inhibition of a PCSK9. For example and without limitation, the compositions described herein can be used to treat lipidemias, e.g., hyperlipidemias and other forms of lipid imbalance such as hypercholesterolemia, hypertriglyceridemia and the pathological conditions associated with these disorders such as heart and circulatory diseases.

Other diseases and conditions that can be modulated by down regulating PCSK9 gene expression include lysosomal storage diseases including, but not limited to, Niemann-Pick disease, Tay-Sachs disease, Lysosomal acid lipase deficiency, and Gaucher Disease. The methods include administering to the subject a therapeutically effective amount or prophylactically effective amount of a compound or composition of the disclosure. In some embodiments, the method includes administering an effective amount of a compound of Formula (V) or a pharmaceutically acceptable salt or ester thereof, or a composition thereof comprising a PCSK9-targeting siRNA to a subject in need thereof, e.g., to a patient having a heterozygous LDLR genotype.

In some embodiments, the effect of the decreased PCSK9 gene results in a decrease in LDL-C(low density lipoprotein cholesterol) levels in the blood, and more particularly in the serum, of a subject, e.g., a mammal, e.g., a human. In some embodiments, LDL-C levels are decreased by at least 10%, 15%, 20%, 25%, 30%, 40%, 50%, 60%, 70%, 80%, 90% or more, as compared to pre-treatment levels.

In some embodiments, there are provided methods for treating or preventing hypercholesterolemia in a subject in need thereof, comprising administering a therapeutically effective amount of a compound or composition of the disclosure to the subject, such that hypercholesterolemia is treated or prevented. The type of hypercholesterolemia is not limited and may be, for example, familial hypercholesterolemia, such as heterozygous familial hypercholesterolemia, or non-familial hypercholesterolemia, such as hypercholesterolemia associated with atherosclerotic or cardiovascular disease.

In some embodiments, there are provided methods for treating or preventing atherosclerotic cardiovascular disease in a subject in need thereof, comprising administering a therapeutically effective amount of a compound or composition of the disclosure to the subject, such that atherosclerotic cardiovascular disease is treated or prevented.

In some embodiments, there are provided methods for treating or preventing type 2 diabetes in a subject in need thereof, comprising administering a therapeutically effective amount of a compound or composition of the disclosure to the subject, such that type 2 diabetes is treated or prevented.

In some embodiments, there are provided methods for reducing serum cholesterol levels in a subject in need thereof, comprising administering a therapeutically effective amount of a compound or composition of the disclosure to the subject, such that serum cholesterol levels are reduced in the subject. In some embodiments, there are provided methods for reducing low-density lipoprotein cholesterol (LDL-C) levels in a subject in need thereof, comprising administering a therapeutically effective amount of a compound or composition of the disclosure to the subject, such that low-density lipoprotein cholesterol (LDL-C) levels are reduced in the subject. In some such embodiments, the subject suffers from hypercholesterolemia, dyslipidemia, hyperlipidemia, atherosclerotic cardiovascular disease, or type 2 diabetes.

In some embodiments, there are provided methods for treating or preventing high cholesterol in a subject, comprising administering a therapeutically effective amount of a compound or composition of the disclosure to the subject, such that high cholesterol is treated or prevented. In some such embodiments, the methods also comprise the step of determining the subject's serum cholesterol level. Serum cholesterol levels may be determined before, during and/or after administration of the compound or composition of the disclosure.

Compounds and compositions of the disclosure may be administered to a subject using any mode of administration known in the art, including, but not limited to subcutaneous, intravenous, intramuscular, intraocular, intrabronchial, intrapleural, intraperitoneal, intraarterial, lymphatic, cerebrospinal, and any combinations thereof. In some embodiments, the agents are administered subcutaneously. In some embodiments, the administration is via a depot injection. A depot injection may release the compound in a consistent way over a prolonged time period. Thus, a depot injection may reduce the frequency of dosing needed to obtain a desired effect, e.g., a desired inhibition of PCSK9, or a therapeutic or prophylactic effect. A depot injection may also provide more consistent serum concentrations. Depot injections may include subcutaneous injections or intramuscular injections. In some embodiments, the depot injection is a subcutaneous injection.

In some embodiments, administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions.

In some embodiments, the infusion pump is a subcutaneous infusion pump. In other embodiments, the pump is a surgically implanted pump that delivers the compound or composition to the liver.

Other modes of administration include epidural, intracerebral, intracerebroventricular, nasal administration, intraarterial, intracardiac, intraosseous infusion, intrathecal, and intravitreal, and pulmonary. The mode of administration may be chosen based upon whether local or systemic treatment is desired and based upon the area to be treated. The route and site of administration may be chosen to enhance targeting.

In some embodiments, the methods of the present disclosure include administering a compound of the disclosure comprising a PCSK9 siRNA (i.e., a PCSK9-targeting siRNA) in a dose sufficient to depress levels of PCSK9 mRNA for at least 5, or 7, 10, 14, 21, 25, 30 or 40 days; and optionally, administering a second single dose of the compound, wherein the second single dose is administered at least 5, or 7, 10, 14, 21, 25, 30 or 40 days after the first single dose is administered, thereby inhibiting expression of the PCSK9 gene in a subject.

In one embodiment, doses of a compound of Formula I comprising a PCSK9 siRNA are administered not more than once every four weeks, not more than once every three weeks, not more than once every two weeks, or not more than once every week. In another embodiment, the administrations can be maintained for one, two, three, or six months, or one year or longer.

In another embodiment, administration can be provided when Low Density Lipoprotein cholesterol (LDL-C) levels reach or surpass a predetermined minimal level, such as greater than 70 mg/dL, 130 mg/dL, 150 mg/dL, 200 mg/dL, 300 mg/dL, or 400 mg/dL.

In general, the PCSK9-targeting siRNA of the compounds of the disclosure does not activate the immune system, e.g., it does not increase cytokine levels, such as TNF-alpha or IFN-alpha levels. For example, when measured by an assay, such as an in vitro PBMC assay, the increase in levels of TNF-alpha or IFN-alpha, is less than 30%, 20%, or 10% of control cells treated with a control dsRNA, such as a dsRNA that does not target PCSK9. For example, a subject can be administered a therapeutic amount of a compound, such as 0.5 mg/kg, 1.0 mg/kg, 1.5 mg/kg, 2.0 mg/kg, or 2.5 mg/kg. The compound can be administered by intravenous infusion over a period of time, such as over a 5 minute, 10 minute, 15 minute, 20 minute, or 25 minute period. The administration is repeated, for example, on a regular basis, such as biweekly (i.e., every two weeks) for one month, two months, three months, four months or longer. After an initial treatment regimen, the treatments can be administered on a less frequent basis. For example, after administration biweekly for three months, administration can be repeated once per month, for six months or a year or longer.

In some embodiments, administration of the compound can reduce PCSK9 levels, e.g., in a cell, tissue, blood, urine or other compartment of the patient by at least 10%, at least 15%, at least 20%, at least 25%, at least 30%, at least 40%, at least 50%, at least 60%, at least 70%, at least 80% or at least 90% or more. In some embodiments, before administration of a full dose of the compound or composition, patients can be administered a smaller dose, such as a 5%>infusion reaction, and monitored for adverse effects, such as an allergic reaction, or for elevated lipid levels or blood pressure. In another example, the patient can be monitored for unwanted immunostimulatory effects, such as increased cytokine (e.g., TNF-alpha or INF-alpha) levels.

A treatment or preventive effect is generally evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given compound or composition can also be judged using an experimental animal model for the given disease as known in the art. When using an experimental animal model, efficacy of treatment is evidenced when a statistically significant reduction in a marker or symptom is observed.

In one embodiment, the compound is administered at a dose of between about 0.25 mg/kg to about 50 mg/kg, e.g., between about 0.25 mg/kg to about 0.5 mg/kg, between about 0.25 mg/kg to about 1 mg/kg, between about 0.25 mg/kg to about 5 mg/kg, between about 0.25 mg/kg to about 10 mg/kg, between about 1 mg/kg to about 10 mg/kg, between about 5 mg/kg to about 15 mg/kg, between about 10 mg/kg to about 20 mg/kg, between about 15 mg/kg to about 25 mg/kg, between about 20 mg/kg to about 30 mg/kg, between about 25 mg/kg to about 35 mg/kg, or between about 40 mg/kg to about 50 mg/kg.

In some embodiments, the compound is administered at a dose of about 0.25 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 1 1 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 2 1 mg/kg, about 22 mg/kg, about 23 mg/kg, about 24 mg/kg, about 25 mg/kg, about 26 mg/kg, about 27 mg/kg, about 28 mg/kg, about 29 mg/kg, 30 mg/kg, about 3 1 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 4 1 mg/kg, about 42 mg/kg, about 43 mg/kg, about 44 mg/kg, about 45 mg/kg, about 46 mg/kg, about 47 mg/kg, about 48 mg/kg, about 49 mg/kg or about 50 mg/kg. In one embodiment, the compound is administered at a dose of about 25 mg/kg.

It should be understood that the dosage or amount of a compound and/or composition used, alone or in combination with one or more active compounds to be administered, depends on the individual case and is, as is customary, to be adapted to the individual circumstances to achieve an optimum effect. Dosing and administration regimens are within the purview of the skilled artisan, and appropriate doses depend upon a number of factors within the knowledge of the ordinarily skilled physician, veterinarian, or researcher (e.g., see Wells et al. eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000)). For example, dosing and administration regimens may depend on the nature and the severity of the disorder to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the therapeutic molecule(s).

Thus the dose(s) of a compound or composition will vary depending upon a variety of factors including, but not limited to: the activity, biological and pharmacokinetic properties and/or side effects of the compound being used; the age, body weight, general health, gender, and diet of the subject; the time of administration, the route of administration, the rate of excretion, and any drug combination, if applicable; the effect which the practitioner desires the compound to have upon the subject; and the properties of the compound being administered (e.g. bioavailability, stability, potency, toxicity, etc). Such appropriate doses may be determined as known in the art. When one or more of the compounds of the disclosure is to be administered to humans, a physician may for example, prescribe a relatively low dose at first, subsequently increasing the dose until an appropriate response is obtained.

The dose of a compound of the disclosure that is administered to a subject may be tailored to balance the risks and benefits of a particular dose, for example, to achieve a desired level of PCSK9 gene suppression (as assessed, e.g., based on PCSK9 mRNA suppression, PCSK9 protein expression, or a reduction in lipid levels) or a desired therapeutic or prophylactic effect, while at the same time avoiding undesirable side effects.

In some embodiments, the compound or composition is administered in two or more doses. If desired to facilitate repeated or frequent infusions, implantation of a delivery device, e.g., a pump, semi-permanent stent (e.g., intravenous, intraperitoneal, intracisternal or intracapsular), or reservoir may be advisable. In some embodiments, the number or amount of subsequent doses is dependent on the achievement of a desired effect, e.g., the suppression of a PCSK9 gene, or the achievement of a therapeutic or prophylactic effect, e.g., reducing a symptom of hypercholesterolemia.

In some embodiments, the compound or composition is administered according to a schedule. For example, the compound or composition may be administered once per week, twice per week, three times per week, four times per week, or five times per week. In some embodiments, the schedule involves regularly spaced administrations, e.g., hourly, every four hours, every six hours, every eight hours, every twelve hours, daily, every 2 days, every 3 days, every 4 days, every 5 days, weekly, biweekly, or monthly. In other embodiments, the schedule involves closely spaced administrations followed by a longer period of time during which the agent is not administered. For example, the schedule may involve an initial set of doses that are administered in a relatively short period of time (e.g., about every 6 hours, about every 12 hours, about every 24 hours, about every 48 hours, or about every 72 hours) followed by a longer time period (e.g., about 1 week, about 2 weeks, about 3 weeks, about 4 weeks, about 5 weeks, about 6 weeks, about 7 weeks, or about 8 weeks) during which the compound or composition is not administered. In one embodiment, the compound is initially administered hourly and is later administered at a longer interval (e.g., daily, weekly, biweekly, or monthly). In another embodiment, the compound is initially administered daily and is later administered at a longer interval (e.g., weekly, biweekly, or monthly). In certain embodiments, the longer interval increases over time or is determined based on the achievement of a desired effect. In a specific embodiment, the compound is administered once daily during a first week, followed by weekly dosing starting on the eighth day of administration. In another specific embodiment, the compound is administered every other day during a first week followed by weekly dosing starting on the eighth day of administration.

In one embodiment, the compound is administered two times per week. In one embodiment, the compound is administered two times per week at a dose of 1 mg/kg. In another embodiment, the compound is administered two times per week at a dose of 2 mg/kg. In one embodiment, the compound is administered once every two weeks. In one embodiment, the compound is administered once every two weeks at a dose of 1 mg/kg. In another embodiment, the compound is administered once every two weeks at a dose of 2 mg/kg.

In one embodiment, the compound is administered once a week. In one embodiment, the compound is administered once a week at a dose of 0.5 mg/kg. In one embodiment, the compound is administered once a week at a dose of 1 mg/kg. In another embodiment, the compound is administered once a week at a dose of 2 mg/kg.

In some embodiments, the compound or composition is administered in a dosing regimen that includes a “loading phase” of closely spaced administrations that may be followed by a “maintenance phase”, in which the compound or composition is administered at longer spaced intervals. In one embodiment, the loading phase comprises five daily administrations of the compound or composition during the first week. In another embodiment, the maintenance phase comprises one or two weekly administrations of the compound or composition. In a further embodiment, the maintenance phase lasts for 5 weeks. In one embodiment, the loading phase comprises administration of a dose of 2 mg/kg, 1 mg/kg or 0.5 mg/kg five times a week. In another embodiment, the maintenance phase comprises administration of a dose of 2 mg/kg, 1 mg/kg or 0.5 mg/kg once, twice, or three times weekly, once every two weeks, once every three weeks, once a month, once every two months, once every three months, once every four months, once every five months, or once every six months. Any of these schedules may optionally be repeated for one or more iterations. The number of iterations may depend on the achievement of a desired effect, e.g., the suppression of a PCSK9 gene, and/or the achievement of a therapeutic or prophylactic effect, e.g., reducing serum cholesterol levels or reducing a symptom of hypercholesterolemia.

In further embodiments, a compound or composition of the disclosure is administered in combination with an additional therapeutic agent. The compound or composition of the disclosure and an additional therapeutic agent can be administered in combination in the same composition, e.g., parenterally, or the additional therapeutic agent can be administered as part of a separate composition or by another method described herein.

Examples of additional therapeutic agents include those known to treat an agent known to treat a lipid disorders, such as hypercholesterolemia, atherosclerosis or dyslipidemia. For example, a compound of the disclosure comprising a PCSK9-targeting siRNA can be administered with, e.g., an HMG-CoA reductase inhibitor (e.g., a statin), a fibrate, a bile acid sequestrant, niacin, an antiplatelet agent, an angiotensin converting enzyme inhibitor, an angiotensin II receptor antagonist (e.g., losartan potassium, such as Merck & Co.'s Cozaar®), an acylCoA cholesterol acetyltransferase (ACAT) inhibitor, a cholesterol absorption inhibitor, a cholesterol ester transfer protein (CETP) inhibitor, a microsomal triglyceride transfer protein (MTTP) inhibitor, a cholesterol modulator, a bile acid modulator, a peroxisome proliferation activated receptor (PPAR) agonist, a gene-based therapy, a composite vascular protectant (e.g., AGT1067, from Atherogenics), a glycoprotein Ilb/IIIa inhibitor, aspirin or an aspirin like compound, an IBAT inhibitor (e.g., S-8921, from Shionogi), a squalene synthase inhibitor, or a monocyte chemoattractant protein (MCP)-I inhibitor.

Exemplary HMG-CoA reductase inhibitors include atorvastatin (Pfizer's Lipitor®/Tahor/Sortis/Torvast/Cardyl), pravastatin (Bristol-Myers Squibb's Pravachol, Sankyo's Mevalotin/Sanaprav), simvastatin (Merck's Zocor®/Sinvacor, Boehringer Ingelheim's Denan, Banyu's Lipovas), lovastatin (Merck's Mevacor/Mevinacor, Bexal's Lovastatina, Cepa; Schwarz Pharma's Liposcler), fluvastatin (Novartis' Lescol®/Locol/Lochol, Fujisawa's Cranoc, Solvay's Digaril), cerivastatin (Bayer's Lipobay/GlaxoSmithKline's Baycol), rosuvastatin (AstraZeneca's Crestor®), and pitivastatin (itavastatin/risivastatin) (Nissan Chemical, Kowa Kogyo, Sankyo, and Novartis).

Exemplary fibrates include, e.g., bezafibrate (e.g., Roche's Befizal®/Cedur®/Bezalip®, Kissei's Bezatol), clofibrate (e.g., Wyeth's Atromid-S®), fenofibrate (e.g., Founder's Lipidil/Lipantil, Abbott's Tricor®, Takeda's Lipantil, generics), gemfibrozil (e.g., Pfizer's Lopid/Lipur) and ciprofibrate (Sanofi-Synthelabo's Modalim®).

Exemplary bile acid sequestrants include, e.g., cholestyramine (Bristol-Myers Squibb's Questran® and Questran Light™), colestipol (e.g., Pharmacia's Colestid), and colesevelam (Genzyme/Sankyo's WelChol™)

Exemplary niacin therapies include, e.g., immediate release formulations, such as Aventis' Nicobid, Upsher-Smith's Niacor, Aventis' Nicolar, and Sanwakagaku's Perycit. Niacin extended release formulations include, e.g., Kos Pharmaceuticals' Niaspan and Upsher-Smith's SIo- Niacin. Exemplary antiplatelet agents include, e.g., aspirin (e.g., Bayer's aspirin), clopidogrel (Sanofi-Synthelabo/Bristol-Myers Squibb's Plavix), and ticlopidine (e.g., Sanofi-Synthelabo's Ticlid and Daiichi's Panaldine).

Other aspirin-like compounds useful in combination with an siRNA targeting PCSK9 include, e.g., Asacard (slow-release aspirin, by Pharmacia) and Pamicogrel (Kanebo/Angelini Ricerche/CEPA). Exemplary angiotensin-converting enzyme inhibitors include, e.g., ramipril (e.g., Aventis' Altace) and enalapril (e.g., Merck & Co.'s Vasotec). Exemplary acyl CoA cholesterol acetyltransferase (AC AT) inhibitors include, e.g., avasimibe (Pfizer), eflucimibe (BioMsrieux Pierre Fabre/Eli Lilly), CS-505 (Sankyo and Kyoto), and SMP-797 (Sumito). Exemplary cholesterol absorption inhibitors include, e.g., ezetimibe (Merck/Schering-Plough Pharmaceuticals Zetia®) and Pamaqueside (Pfizer). Exemplary CETP inhibitors include, e.g., Torcetrapib (also called CP-529414, Pfizer), JTT-705 (Japan Tobacco), and CETi-I (Avant Immunotherapeutics). Exemplary microsomal triglyceride transfer protein (MTTP) inhibitors include, e.g., implitapide (Bayer), R-103757 (Janssen), and CP-346086 (Pfizer). Other exemplary cholesterol modulators include, e.g., NO- 1886 (Otsuka/TAP Pharmaceutical), CT1027 (Pfizer), and WAY- 135433 (Wyeth-Ayerst).

Exemplary bile acid modulators include, e.g., HBS-107 (Hisamitsu/Banyu), Btg-511 (British Technology Group), BARI-1453 (Aventis), S-8921 (Shionogi), SD-5613 (Pfizer), and AZD- 7806 (AstraZeneca). Exemplary peroxisome proliferation activated receptor (PPAR) agonists include, e.g., tesaglitazar (AZ-242) (AstraZeneca), Netoglitazone (MCC-555) (Mitsubishi/Johnson & Johnson), GW-409544 (Ligand Pharmaceuticals/GlaxoSmithKline), GW-501516 (Ligand Pharmaceuticals/GlaxoSmithKline), LY-929 (Ligand Pharmaceuticals and Eli Lilly), LY-465608 (Ligand Pharmaceuticals and Eli Lilly), LY-5 18674 (Ligand Pharmaceuticals and Eli Lilly), and MK-767 (Merck and Kyorin). Exemplary gene-based therapies include, e.g. AdGWEGF 121.10 (GenVec), ApoAl (UCB Pharma/Groupe Fournier), EG-004 (Trinam) (Ark Therapeutics), and ATP-binding cassette transporter-Al (ABCA1) (CV Therapeutics/Incyte, Aventis, Xenon). Exemplary Glycoprotein Ilb/IIIa inhibitors include, e.g., roxifiban (also called DMP754, Bristol-Myers Squibb), Gantofiban (Merck KGaA/Yamanouchi), and Cromafiban (Millennium Pharmaceuticals). Exemplary squalene synthase inhibitors include, e.g., BMS- 1884941 (Bristol-Myers Squibb), CP-210172 (Pfizer), CP-295697 (Pfizer), CP-294838 (Pfizer), and TAK-475 (Takeda). An exemplary MCP-I inhibitor is, e.g., RS-504393 (Roche Bioscience). The anti-atherosclerotic agent BO-653 (Chugai Pharmaceuticals), and the nicotinic acid derivative Nyclin (Yamanouchi Pharmacuticals) are also appropriate for administering in combination with a siRNA targeting PCSK9.

Exemplary combination therapies suitable for administration with a siRNA targeting PCSK9 include, e.g., advicor (Niacin/lovastatin from Kos Pharmaceuticals), amlodipine/atorvastatin (Pfizer), and ezetimibe/simvastatin (e.g., Vytorin® 10/10, 10/20, 10/40, and 10/80 tablets by Merck/Schering-Plough Pharmaceuticals).

Agents for treating hypercholesterolemia, and suitable for administration in combination with an siRNA targeting PCSK9 include, e.g., lovastatin, niacin Altoprev® Extended-Release Tablets (Andrx Labs), lovastatin Caduet® Tablets (Pfizer), amlodipine besylate, atorvastatin calcium Crestor® Tablets (AstraZeneca), rosuvastatin calcium Lescol® Capsules (Novartis), fluvastatin sodium Lescol® (Reliant, Novartis), fluvastatin sodium Lipitor® Tablets (Parke-Davis), atorvastatin calcium Lofibra® Capsules (Gate), Niaspan Extended-Release Tablets (Kos), niacin Pravachol Tablets (Bristol-Myers Squibb), pravastatin sodium TriCor® Tablets (Abbott), fenofibrate Vytorin® 10/10 Tablets (Merck/Schering-Plough Pharmaceuticals), ezetimibe, simvastatin WelChol™ Tablets (Sankyo), colesevelam hydrochloride Zetia® Tablets (Schering), ezetimibe Zetia® Tablets (Merck/Schering-Plough Pharmaceuticals), and ezetimibe Zocor® Tablets (Merck).

In one embodiment, a compound of the disclosure comprising a PCSK9 targeting siRNA is administered in combination with anezetimibe/simvastatin combination (e.g., Vytorin® (Merck/Schering-Plough Pharmaceuticals)). In one embodiment, the compound is administered to the patient, and then the additional therapeutic agent is administered to the patient (or vice versa). In another embodiment, the compound of the disclosure and the additional therapeutic agent are administered at the same time.

In another embodiment, there is provided a method of instructing an end user, e.g., a caregiver or a subject, on how to administer a compound or composition described herein. The method includes, optionally, providing the end user with one or more doses of the compound or composition, and instructing the end user to administer the compound on a regimen described herein, thereby instructing the end user.

In an embodiment, there is provided a method of treating a patient by selecting a patient on the basis that the patient is in need of LDL lowering, LDL lowering without lowering of HDL, ApoB lowering, or total cholesterol lowering. The method includes administering to the patient a compound or composition of the disclosure in an amount sufficient to lower the patient's LDL levels or ApoB levels, e.g., without substantially lowering HDL levels.

Genetic predisposition plays a role in the development of target gene associated diseases, e.g., hyperlipidemia. Therefore, a patient in need of a compound or composition of the disclosure can be identified by taking a family history, or, for example, screening for one or more genetic markers or variants. Examples of genes involved in hyperlipidemia include but are not limited to, e.g., LDL receptor (LDLR), the apoliproteins (ApoAl, ApoB, ApoE, and the like), Cholesteryl ester transfer protein (CETP), Lipoprotein lipase (LPL), hepatic lipase (LIPC), Endothelial lipase (EL), and Lecithinxholesteryl acyltransferase (LCAT). A healthcare provider, such as a doctor, nurse, or family member, can take a family history before prescribing or administering a PCSK9-targeting compound or composition of the disclosure. In addition, a test may be performed to determine a genotype or phenotype. For example, a DNA test may be performed on a sample from the patient, e.g., a blood sample, to identify the PCSK9 genotype and/or phenotype before a PCSK9 dsRNA is administered to the patient.

In another embodiment, a test is performed to identify a related genotype and/or phenotype, e.g., a LDL receptor (LDLR) genotype. Example of genetic variants with the LDLR gene can be found in the art, e.g., in the following publications which are incorporated by reference: Costanza et al (2005) Am J Epidemiol. 15;161(8):714-24; Yamada et al. (2008) J Med Genet. Jan;45(1):22-8, Epub 2007 Aug 31; and Boes et al (2009) Exp. Gerontol 44: 136-160, Epub 2008 Nov 17.

“Treating” or “treatment” of any disease or disorder refers, in some embodiments, to ameliorating at least one disease or disorder. In certain embodiments “treating” or “treatment” refers to ameliorating at least one physical parameter, which may or may not be discernible by the patient. In certain embodiments, “treating” or “treatment” refers to inhibiting the disease or disorder, either physically (e.g., stabilization of a discernible symptom), physiologically (e.g., stabilization of a physical parameter), or both. In some embodiments, “treating” or “treatment” refers to improving the quality of life or reducing the symptoms or side effects of a disease in a subject in need thereof “Therapeutically effective amount” means the amount of a compound that, when administered to a subject for treating or preventing a disease, is sufficient to effect such treatment or prevention of the disease. The “therapeutically effective amount” will vary depending on the compound, the disease and its severity, and the age, weight, etc., of the subject having the disease to be treated or prevented. As used herein, the term “therapeutically effective amount” refers to an amount of a compound or composition sufficient to prevent, treat, inhibit, reduce, ameliorate or eliminate one or more causes, symptoms, or complications of a disease or disorder such as, for example, hypercholesteremia. The terms “effective amount” and “therapeutically effective amount” are used interchangeably herein.

“Preventing” or “prevention” or “prophylaxis” of any disease or disorder refers, in some embodiments, to at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease).

The term “subject” includes animals, including mammals and humans, particularly humans. Non-limiting examples of subjects include humans, monkeys, cows, rabbits, sheep, goats, pigs, dogs, cats, rats, mice, and transgenic species thereof.

In some embodiments of methods of the disclosure, methods are provided for treating a subject with a disease mediated by PCSK9 expression, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of ANGPTL3, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of ApoB, comprising administering a therapeutically effective amount of a compound or composition of the disclosure to the subject.

In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of ApoC, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of SCD1, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of FVII, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of p53, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of HBV, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

In some embodiments of methods of the disclosure, methods are provided for treating a subject with a disease mediated by expression of HCV, comprising administering a therapeutically effective amount of a compound or composition of the disclosure to the subject. In some such embodiments, the subject has hypercholesterolemia, dyslipidemia, or hyperlipidemia. In some embodiments, after administration of the compound or composition of the disclosure, the serum cholesterol level of the subject is reduced. In some embodiments, after administration of the compound or composition of the disclosure, the LDL-C level of the subject is reduced.

### Examples

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention. Unless otherwise specified, the materials and instruments used in the present disclosure are generally commercially available.

### Example 10 Prepaaraation of Intermediates M13 and S2A.

Intermediate M13 was synthesized as described (T. P. Prakash, M. J. Graham, P. P. Seth, etc. Nucleic Acids Research, 2014, 42, 8796-8807).

Intermediate S2A was synthesized as described (N. Hebert, P. W. Davis, E. L. D. Baets, O. L. Acevedo, Tetrahedron Letter, 1994, 35, 9509-9512).

### Example 2: Preparation of Conjugate L

Conjugate 1 was synthesized according to the route of synthesis below:

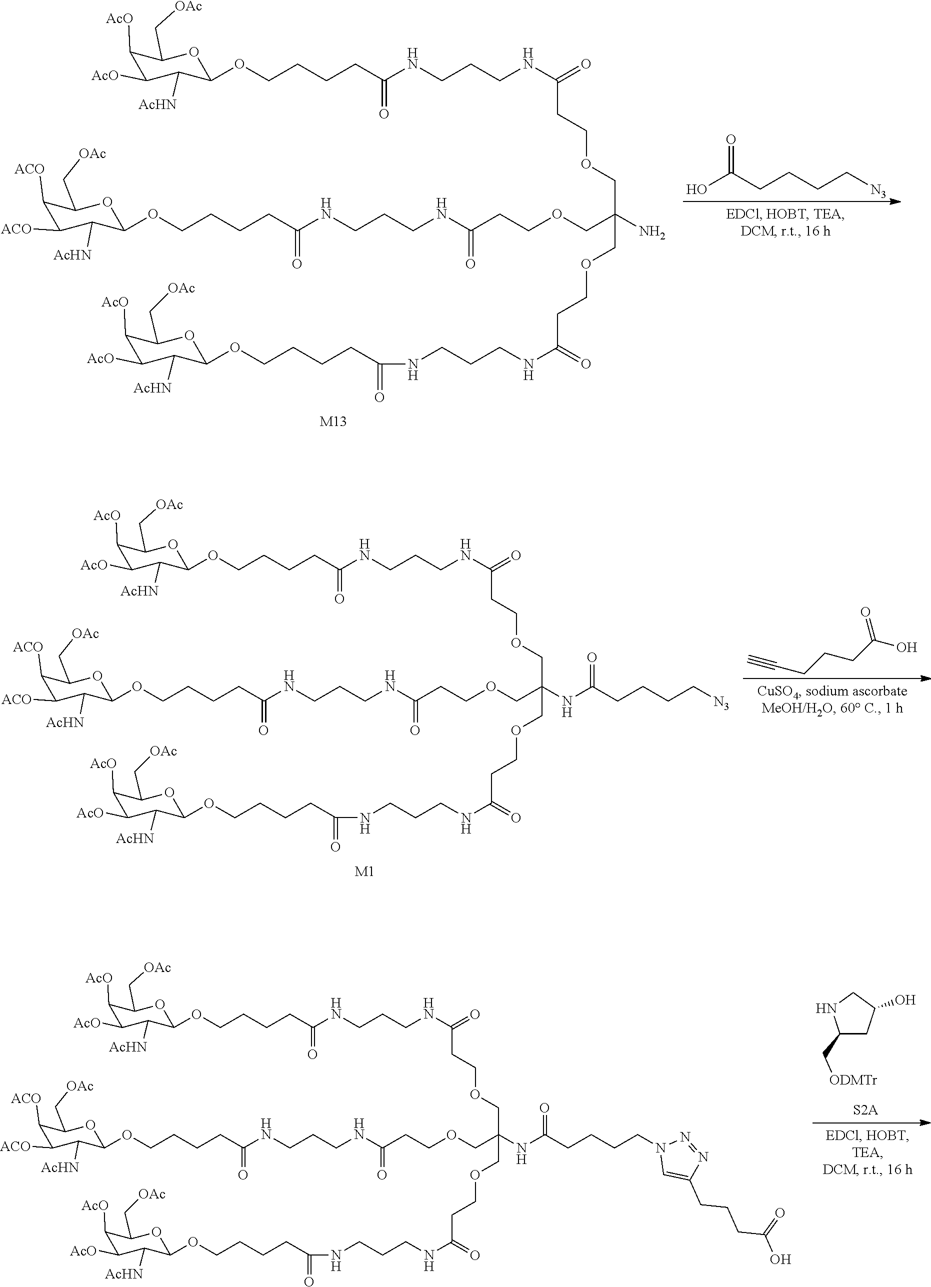

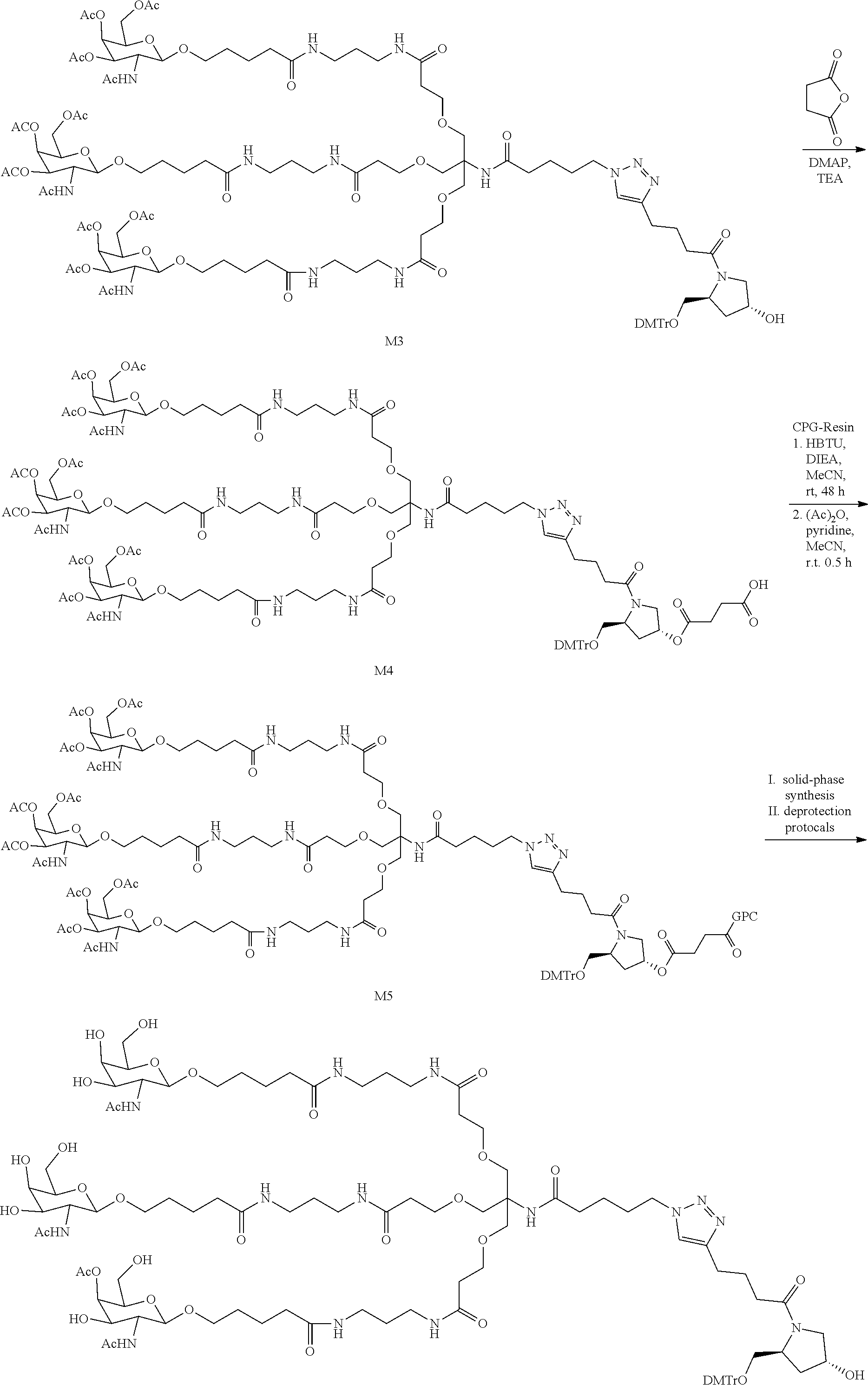

1. Synthesis of intermediate M1. To a solution of 5-azidopentanoic acid (263 mg, 1.84 mmol, 1.5 eq.) in DCM (20 mL) was added EDCI (352.64 mg, 1.84 mmol, 1.5 eq.) and HOBt (248.56 mg, 1.84 mmol, 1.5 eq.). After stirring at 25° C. for 15 min under N₂, M13 (2.2 g, 1.23 mmol, 1 eq.) was added and the resulting mixture was stirred for 16 h at 25° C. under N₂. TLC (DCM:MeOH=8:1) showed the reactant was completely consumed. The reaction mixture was quenched with H₂O (10 mL) and extracted with DCM (10 mL*3). The combined organic phase was washed with brine and dried over anhydrous Na₂SO₄. After filtration, the filtrate was concentrated at 40° C. under reduced pressure. The crude product was purified by column chromatography on silica gel eluted with MeOH/DCM (0:100˜15:100), providing M1 (1.1 g, 573.21 μmol, 46.74% yield) as a white solid.

2. Synthesis of intermediate M2. M1 (800 mg, 416.88 μmol, 1 eq.), hex-5-ynoic acid (46.74 mg, 416.88 μmol, 1 eq.) and CuSO₄ (79.84 mg, 500.25 μmol, 1.2 eq.) was added to a stirred mixture of sodium ascorbate (206.47 mg, 1.04 mmol, 2.5 eq.) in MeOH:H₂O=1:1 (2 mL) at room temperature and the mixture was stirred at 60° C. for 1 h. LCMS showed the starting material was consumed and new product was formed. The mixture was filtered, and the solvent was evaporated under reduced pressure. The residue was purified by silica gel column flash chromatography using MeOH/DCM (0˜15%) as eluent, providing M2 (550 mg, 64.9% yield) as a yellow solid.

3. Synthesis of intermediate M3. To a solution of M2 (350 mg, 172.32 μmol, 1 eq.) in DCM (10 mL) was added EDCI (49.55 mg, 258.47 μmol, 1.5 eq.) and HOBt (34.92 mg, 258.47 μmol, 1.5 eq.). After stirring at 25° C. for 15 min under N₂, S2A (86.75 mg, 206.78 Nmol, 1.2 eq.) was added and the resulting mixture was stirred for 16 h at 25° C. TLC showed the starting material was consumed and new product formed. The reaction mixture was quenched with H₂O (24 mL) and extracted with DCM (24 mL*3). The combined organic phase was washed with brine and dried over anhydrous Na₂SO₄. After filtration, the filtrate was concentrated at 35° C. under reduced pressure. The crude product was purified by column chromatography on silica gel eluted with MeOH/DCM containing 1% TEA (0:100-15:100), providing M3 (350 mg, 143.88 μmol, 83.50% yield) as a yellow solid.

4. Synthesis of intermediate M4. M3 (350 mg, 143.88 μmol, 1 eq.), tetrahydrofuran-2,5-dione (115.18 mg, 1.15 mmol, 8 eq.), DMAP (35.15 mg, 287.75 μmol, 2 eq.) and TEA (349.41 mg, 3.45 mmol, 479.96 μL, 24 eq.) were mixed in DCM (10 mL) and stirred at 20° C. for 16 h under N₂ atmosphere. LCMS showed the starting material was consumed and new product formed. The mixture was evaporated under reduced pressure to give crude M4 as a yellow oil. The crude product was purified by preparatory HPLC with MeCN/water containing 0.01% ammonia water, providing M4 (155 mg, 60.88 μmol, 44.06% yield, 99.48% HPLC purity) as a white solid. LCMS (ESI): Cal. for C120H174N₁₄O₄₅: 2531.2, Found [M+H]⁺: 2532.6.

5. Synthesis of intermediate M5. M4 (92 mg, 36.32 μmol, 1 eq.) and HBTU (27.55 mg, 72.65 μmol, 2 eq.) were dissolved in MeCN (8 mL). DIEA (18.78 mg, 145.30 μmol, 25.31 μL, 4 eq.) was added to the solution and the mixture was swirled for 3-4 min followed by addition of CPG-Resin (1.09 g, 50 μmol/g). The suspension was gently shaken at room temperature on a wrist-action shaker for 48 h and then subjected to filtration and washed with MeCN (25 ml) twice. The solid was dried under vacuum for 2 h at 45° C. to give a white solid (1.08 g). The acetyl acetate (3.64 mg, 35.64 μmol, 9.31e-2 eq.) and pyridine (7.77 mg, 98.18 μmol, 7.90 μL, 2.56e-1 eq.) were dissolved in MeCN (10 mL), and the mixture was swirled for 3-4 min followed by addition of the above white solid (1.08 g). The suspension was gently shaken at room temperature on a wrist-action shaker for 30 min and then filtered. The filter cake was washed with MeCN (10 ml) twice. The obtained solid was dried under vacuum for 2 h at 45° C., providing M5 (1.08 g) as a white solid. Loading: 23.3 μmol/g.

6. Synthesis of conjugate 1. Conjugate 1 was obtained from M5 through standard solid-phase oligonucleotide synthesis and de-protection protocols, as described (M. J. Damha, K. K. Ogilvie, Methods Mol. Biol. 1993, 20, 81-114).

### Example 3: Preparation of Conjugate 2

Conjugate 2 was synthesized according to the route of synthesis below:

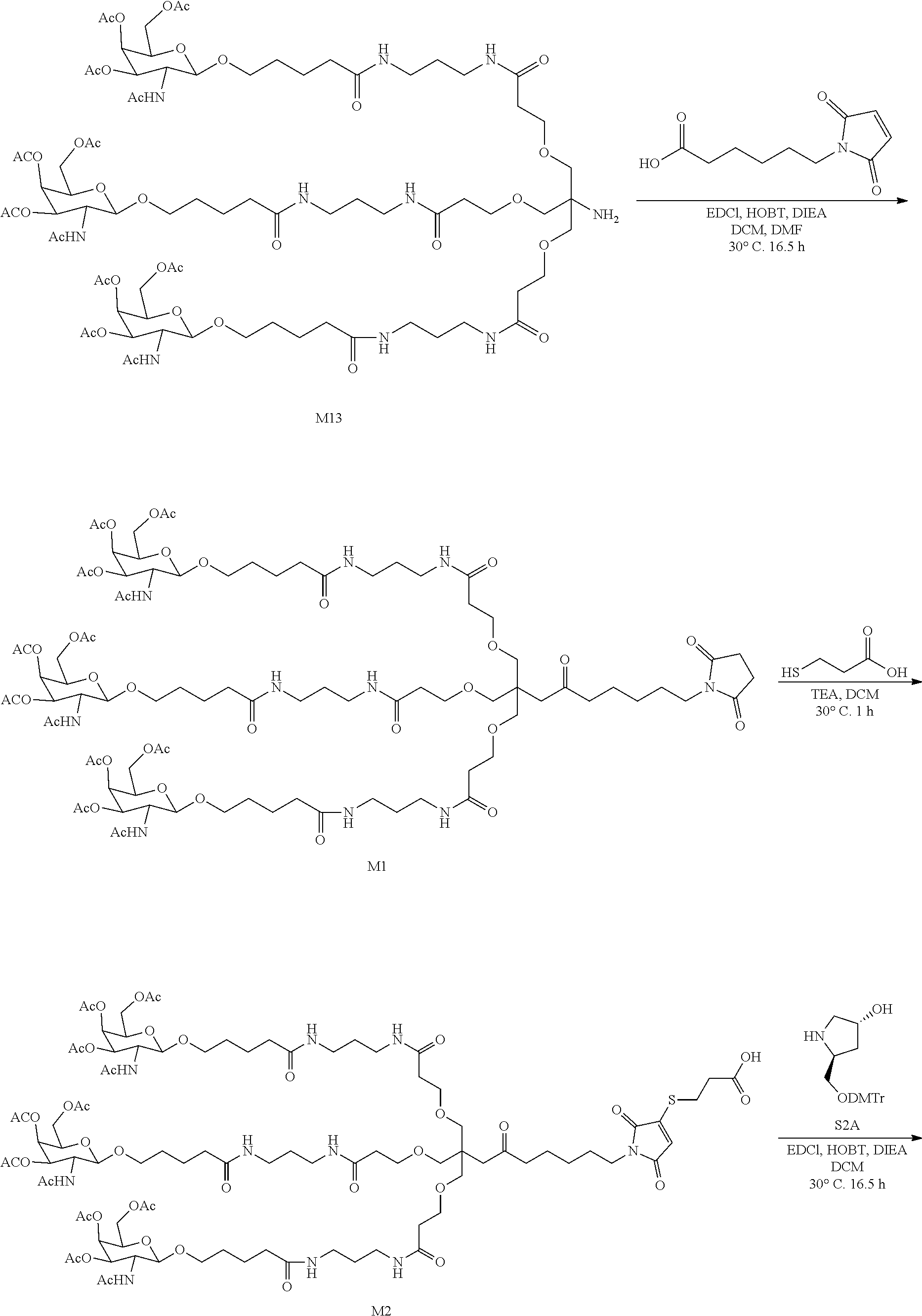

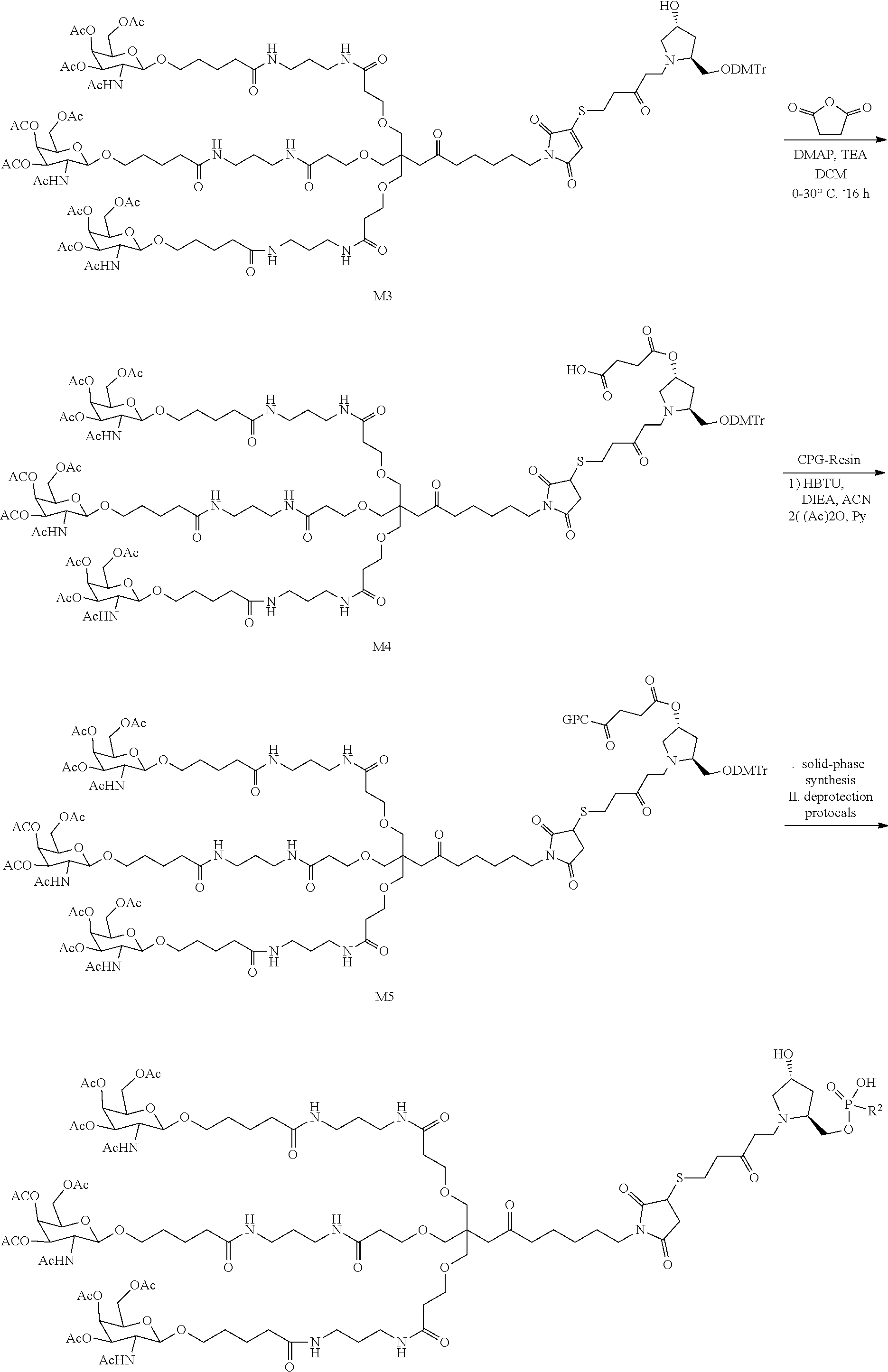

1. Synthesis of intermediate M1. To a solution of HOBt (120.51 mg, 891.91 μmol , 2 eq.) and EDCI (170.98 mg, 891.91 μmol , 2 eq.) in DCM (10 mL) was added DIEA (288.18 mg, 2.23 mmol, 388.38 μL, 5 eq.) and then 6-(2, 5-dioxopyrrol-1-yl)hexanoic acid (145 mg, 686.51 μmol , 1.54 eq.). After stirring for 0.5 h, M13 (800 mg, 445.96 μmol , 1 eq.) in DCM (12 mL) solution was added to the above mixture and the resulting mixture was stirred at 25° C. for 16 h under N₂. The reaction was monitored by TLC and HPLC. The reaction mixture was diluted with DCM (30 mL) and washed with brine (30 mL*2). The water phase was extracted with DCM (20 mL) and the combined organic phase was dried over anhydrous Na₂SO₄. After filtration, the filtrate was evaporated at 35° C. The residue was purified by column chromatography on silica gel eluted with DCM/MeOH (99:1-95:5-90:10-80:20). M1 was obtained as a light yellow solid (613 mg, 246.79 μmol , 80% purity).

2. Synthesis of intermediate M2. To a mixture of M1 (400 mg, 161.04 μmol , 1 eq.) and 3-sulfanylpropanoic acid (17 mg, 161.04 μmol , 14.03 μL, 1 eq.) in DCM (10 mL) was added TEA (49 mg, 483.12 μmol , 67.15 μL, 3 eq.) and the mixture was then stirred at 25° C. for 0.5 h. LC-MS indicated the expected M2 was formed. The reaction mixture was used directly for the next step without further purification.

3. Synthesis of intermediate M3. To a solution of M2 (337 mg, 161.04 μmol , 1 eq.) in DCM (5 mL) was added EDCI (92.61 mg, 483.11 μmol , 3 eq.), HOBt (65.28 mg, 483.11 μmol, 3 eq.) and DIEA in DCM solution (124.88 mg, 966.22 μmol, 168.30 μL, 6 eq., 2 mL) and the mixture was then stirred at 30° C. under N₂ for 0.5 h. After that, S2A (135.11 mg, 322.07 μmol, 2 eq.) in DCM (3 mL) solution was added and the resulting mixture was stirred at 30° C. for 16 h. LC-MS indicated the expected product was formed. The reaction mixture was concentrated at 35° C. to give the crude product. The crude product was purified by column chromatography on silica gel eluted with DCM/MeOH (99:1-95:5-93:7-90:10). M3 (420 mg, 134.68 μmol) was obtained in 83.6% yield.

4. Synthesis of intermediate M4. To a solution of M3 (420 mg, 117.85 μmol, 1 eq.) in DCM (20 mL) was added DMAP (28.79 mg, 235.70 μmol, 2 eq.), TEA (536.62 mg, 5.30 mmol, 737.12 μL, 45 eq.) and succinic anhydride (176.90 mg, 1.77 mmol, 15 eq.) under an ice bath. The resulting mixture was stirred at 25˜30° C. under N₂ for 16 h. LC-MS indicated the expected product was formed. The reaction mixture was concentrated at 40° C. to give the crude product. The crude product was twice purified by preparatory HPLC. M4 was obtained as a white solid (40 mg, 15.24 μmol, 98.83% purity). LCMS (ESI): Cal. for C122H176N₁₂O₄₇S: 2594.85, Found [M+H]⁺: 2595.7.

5. Synthesis of intermediate M5. M4 (80 mg, 30.83 μmol, 1 eq.) and HBTU (23.38 mg, 61.66 μmol, 2 eq.) were dissolved in MeCN (10 mL). DIEA (15.94 mg, 123.32 Nmol, 21.48 μL, 4 eq.) was added to the solution and the mixture was swirled for 3-4 min followed by addition of CPG-Resin (925.00 mg, 50 μmol/g). The suspension was gently shaken at room temperature on a wrist-action shaker for 48 h. The reaction mixture was filtered and washed with MeCN (25 ml) twice. The solid was dried under vacuum for 2 h at 35° C. to get a white solid (950 mg, 329.60 μmol). Then acetyl acetate (4.85 mg, 47.5 μmol, 4.49 μL, 1.24e-1 eq.) and pyridine (11.27 mg, 142.5 μmol, 11.47 μL, 3.72e-1 eq.) were dissolved in MeCN (10 mL), and the mixture was swirled for 3-4 min followed by addition of the above white solid (950 mg, 327.78 μmol, 8.56e-1 eq.). The suspension was gently shaken at room temperature on a wrist-action shaker for 30 min. The mixture was then subjected to filtration and washed with MeCN (10 ml) twice. The solid was dried under vacuum for 2 h at 40° C., providing M5 (925 mg, 320.92 μmol) as a white solid. Loading: 18.9 umol/g.

6. Synthesis of conjugate 2. Conjugate 2 was obtained from M5 through standard solid-phase oligonucleotide synthesis and de-protection protocols, as described (M. J. Damha, K. K. Ogilvie, Methods Mol. Biol. 1993, 20, 81-114).

### Example 4: Preparation of Conjugate 3

Conjugate 3 was synthesized according to the route of synthesis below:

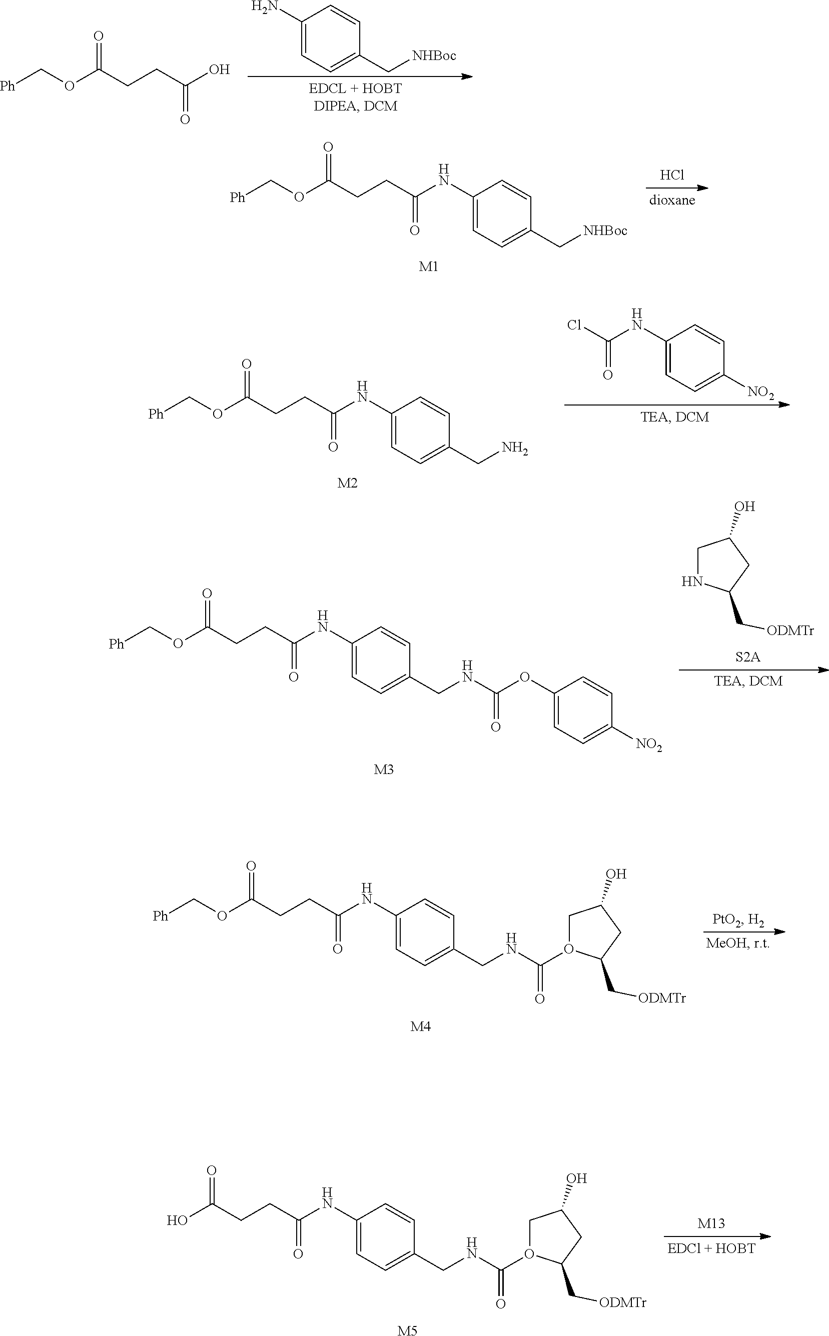

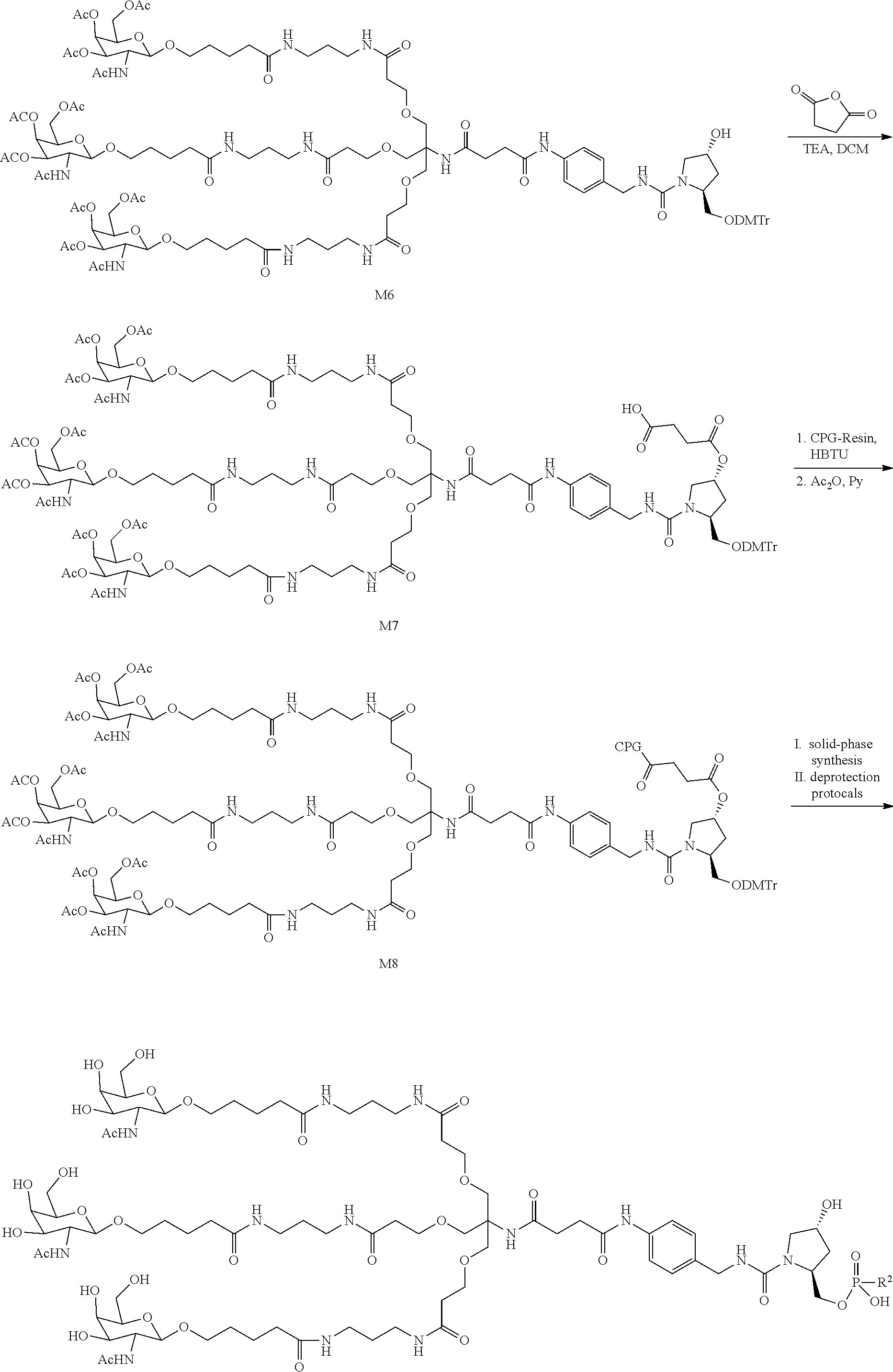

1. Synthesis of intermediate M1. 4-benzyloxy-4-oxo-butanoic acid (1.44 g, 6.93 mmol, 1.1 eq.), EDCI (1.81 g, 9.45 mmol, 1.5 eq.) and HOBT (1.28 g, 9.45 mmol, 1.5 eq.) were dissolved in DCM (30 mL). Then, the tert-butyl N-[(4-aminophenyl) methyl]carbamate (1.4 g, 6.30 mmol, 1 eq.) was added and the mixture was stirred at room temperature for 2 hours. LCMS showed the starting material was consumed and new product formed. Water (15 mL) was added and the mixture extracted with DCM (20 mL) twice. The combined organic fractions were washed with brine, and dried with Na₂SO₄. After filtration, the solvent was evaporated under reduced pressure. The residue was purified by silica gel column flash chromatography using EtOAc/petroleum ether (0˜20%) as eluent, providing M1 (2.90 g, 5.13 mmol, 81.49% yield, 73% purity) as a yellow solid.

2. Synthesis of intermediate M2. M1 (1.0 g, 2.42 mmol, 1 eq.) was dissolved in dioxane (10 mL). HCl in dioxane solution (4 N) (40.00 mmol, 10 mL, 16.5 eq.) was then added and the mixture was stirred at room temperature for 1 hour. LCMS showed the starting material was consumed and new product formed. The mixture was evaporated under reduced pressure to give M2 (840 mg, 2.41 mmol, 99.33% yield, CL) as a yellow solid.

3. Synthesis of intermediate M3. M2 (840 mg, 2.41 mmol, 1 eq., CL) and TEA (731.03 mg, 7.22 mmol, 1.00 mL, 3 eq.) were dissolved in DCM (40 mL). Then, the (4-nitrophenyl) carbonochloridate (970.77 mg, 4.82 mmol, 2 eq.) was added, and the mixture was stirred at room temperature for 1 hour. LCMS showed starting material was consumed and new product formed. Water (30 mL) was added and the mixture was extracted with DCM (30 mL) three times. The combined organic fractions were washed with brine, and dried with Na₂SO₄. After filtration, the filtrate was evaporated under reduced pressure. The residual mixture was purified by recrystallization from DCM (20 ml) to give M3 (800 mg, 1.68 mmol, 69.58% yield) as a white solid.

4. Synthesis of intermediate M4. M3 (600 mg, 1.26 mmol, 1 eq.) and S2A (527 mg, 1.26 mmol, 1 eq.) were dissolved in DCM/THF (4:1, v/v, 25 mL). Then, the TEA (508.64 mg, 5.03 mmol, 698.68 μL, 4 eq.) was added and the mixture was stirred at room temperature for 2 hours. LCMS showed starting material was consumed and new product formed. Water (20 mL) was added and the mixture was extracted with DCM (20 mL) twice. The combined organic fractions were washed with brine, and dried with anhydrous Na₂SO₄. After filtration, the filtrate was evaporated under reduced pressure to give 1.0 g crude product as a yellow solid. The crude product was purified by silica gel column flash chromatography using MeOH/DCM (0-4%) as eluent, providing M4 (800 mg, 1.06 mmol, 84.00% yield) as a yellow solid.

5. Synthesis of intermediate M5. M4 (600.00 mg, 791.69 μmol, 1 eq.) was dissolved in MeOH (20 mL), followed by addition of PtO₂ (257 mg, 1.13 mmol, 1.43 eq.), and the mixture was stirred at 25-30° C. under hydrogen atmosphere (hydrogen balloon) for 3 h. LCMS showed the starting material was consumed and new product formed. After filtration, water (15 mL) was added and the mixture was extracted with DCM (2×20 mL). The combined organic fractions were washed with brine and dried with anhydrous Na₂SO₄. After filtration, the collected filtrate was evaporated under reduced pressure. The residue was purified by silica gel column flash chromatography (MeOH/DCM=0-20%, containing 0.1% TEA) to give M5 (500 mg, 748.79 μmol, 94.58% yield) as a yellow solid.

6. Synthesis of intermediate M6. To a solution of M13 (268.65 mg, 149.76 μmol, 1 eq.) in DCM (10 mL) was added EDCI (57.42 mg, 299.52 μmol, 2.0 eq.) and HOBt (40.47 mg, 299.52 μmol, 2.0 eq.). After stirring at 25° C. for 15 min under N₂, M5 (100 mg, 149.76 μmol, 1 eq.) was added and the resulting mixture was stirred for 16 h at 25° C. LCMS showed the starting material was consumed and new product formed. Water (15 mL) was added and the mixture extracted with DCM (2×10 mL). The combined organic fractions were washed with brine and dried with Na₂SO₄. After filtration, the collected filtrate was evaporated under reduced pressure. The residue was purified by silica gel column flash chromatography using MeOH/DCM (0-5%, containing 0.1% TEA) as eluent, providing M6 (300 mg, 101.90 μmol, 68.04% yield, 83% purity) as a white solid.

7. Synthesis of intermediate M7. M6 (300 mg, 122.77 μmol, 1 eq.), tetrahydrofuran-2,5-dione (122.86 mg, 1.23 mmol, 10 eq.), DMAP (30.00 mg, 245.54 μmol, 2 eq.) and TEA (372.69 mg, 3.68 mmol, 511.93 μL, 30 eq.) were dissolved in DCM (20 mL) and the resulting mixture was stirred at 20° C. for 16 h under N₂ atmosphere. LCMS showed the starting material was consumed and new product formed. The mixture was then evaporated under reduced pressure to give crude M7 as yellow oil. The crude product was purified by Prep-HPLC with MeCN/water containing 0.01% ammonia water, providing M7 (53 mg, 20.84 μmol, 37.86% yield) as a white solid. LCMS (ESI): Cal. for C₁₂₁H₁₇₁N₁₃O₄₆:2542.2, FOUND [M+H]⁺2543.2.

8. Synthesis of intermediate M8. M7 (53 mg, 20.84 μmol, 1 eq.) and HBTU (15.80 mg, 41.67 μmol, 2 eq.) were dissolved in MeCN (10 mL). DIEA (10.77 mg, 83.34 μmol, 14.52 μL, 4 eq.) was added to the solution and the mixture was swirled for 3-4 min followed by addition of CPG-Resin (625 mg, 50 μmol/g). The suspension was gently shaken at room temperature on a wrist-action shaker for 48 h. The mixture was then filtered and washed with MeCN (10 ml) twice. The solid was dried under vacuum for 2 h at 45° C. to give a white solid (620 mg). Acetyl acetate (3.06 mg, 30 μmol, 1.37e-1 eq.) and pyridine (4.44 mg, 56.15 μmol, 4.52 μL, 2.56e-1 eq.) were dissolved in MeCN (10 mL), and the mixture was swirled for 3-4 min followed by addition of the above white solid (620 mg). The suspension was gently shaken at room temperature on a wrist-action shaker for 30 min and then filtered following wash with MeCN (10 ml) twice. The solid was dried under vacuum for 2 h at 45° C., providing M8 (480 mg) as a white solid. Loading: 13.3 μmol/g.

9. Synthesis of conjugate 3. Conjugate 3 was obtained from M8 through standard solid-phase oligonucleotide synthesis and de-protection protocols, as described (M. J. Damha, K. K. Ogilvie, Methods Mol. Biol. 1993, 20, 81-114).

### Example 5: Preparation of Conjugate 4

Conjugate 4 was synthesized according to the route of synthesis below:

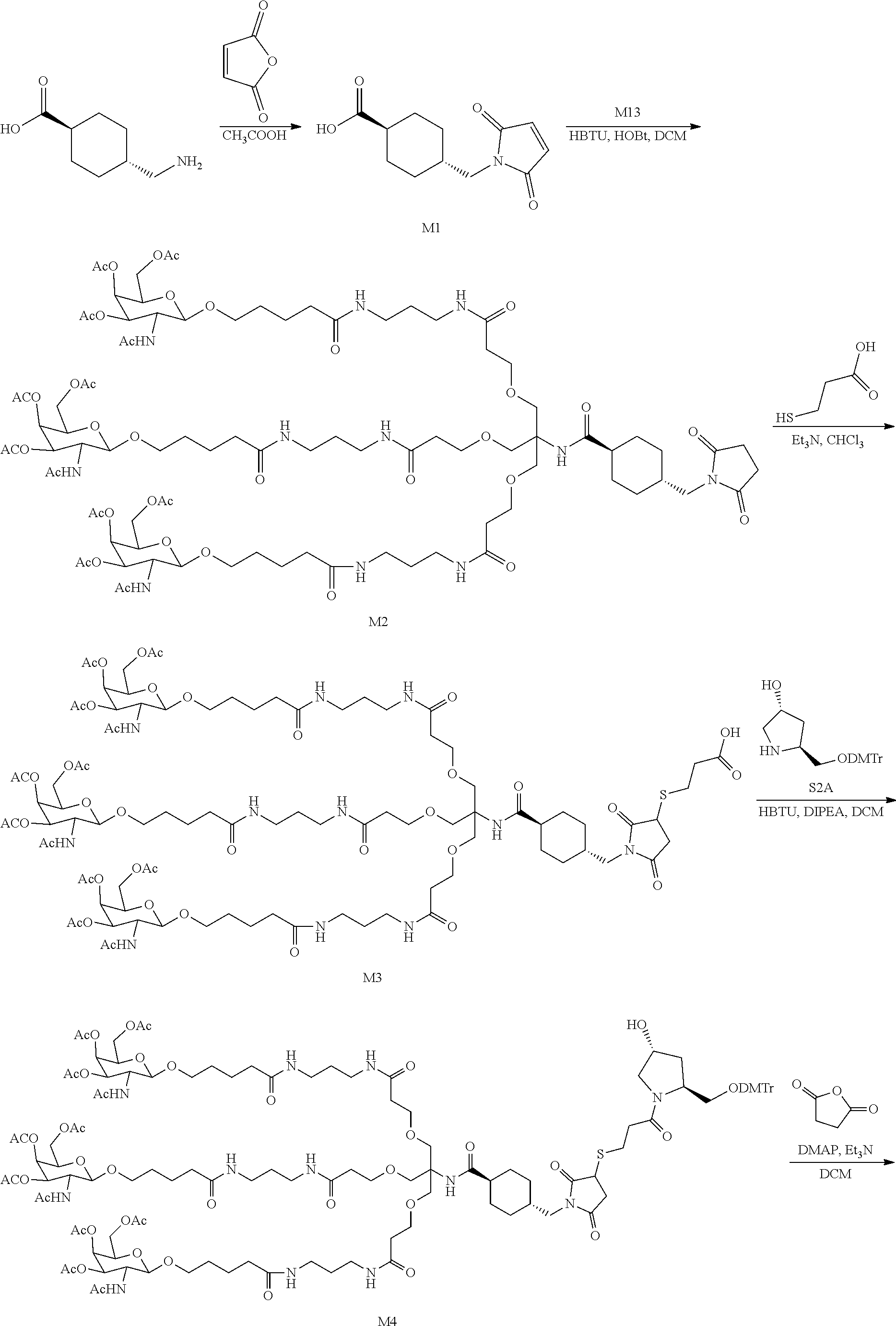

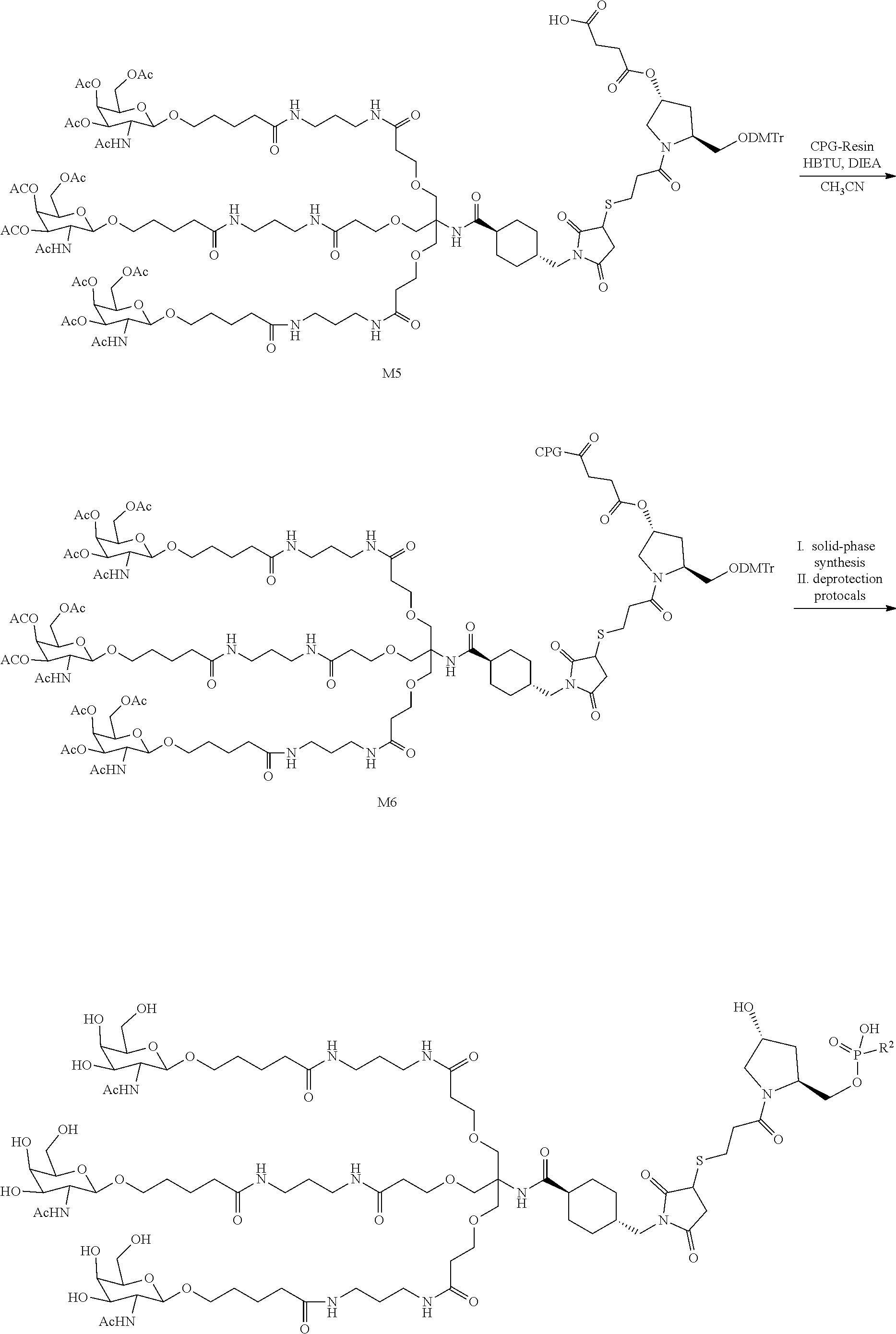

1. Synthesis of intermediate M1. To a mixture of furan-2,5-dione (1 g, 10.2 mmol, 1 eq.) in acetic acid (20 mL) was added 4-(aminomethyl)cyclohexanecarboxylic acid (1.60 g, 10.20 mmol, 1 eq.). The mixture was then stirred at 160° C. for 6 h under N₂ atmosphere. TLC detection indicated the reactant was nearly consumed completely. The solvent was removed under reduced pressure at 60° C. to give the crude product. The crude product was further purified by silica gel using MeOH/DCM (0-4%) as eluent. M1 was obtained (1.1 g, 4.64 mmol, 45.46% yield) as a white solid. ¹H NMR (500 MHz, DMSO-d6) δ 7.03 (s, 2H), 3.27 (d, J=7.0 Hz, 2H), 2.13 (d, J=11.0 Hz, 1H), 1.90 (d, J=13.0 Hz, 2H), 1.65 (d, J=12.5 Hz, 2H), 1.56 (s, 1H), 1.25 (q, J=12.5 Hz, 2H), 0.97 (q, J=12.5 Hz, 2H).

2. Synthesis of intermediate M2. To a solution of M1 (79.35 mg, 334.47 μmol, 1 eq.) in DCM (10 mL) was added HBTU (253.69 mg, 668.93 μmol, 2 eq.), DIPEA (86.45 mg, 668.93 μmol, 116.51 μL, 2 eq.), HOBT (225.97 mg, 1.67 mmol, 5 eq.) and M13 (600 mg, 334.47 μmol, 1 eq.). The resulting mixture was then stirred overnight at r.t. under N₂ atmosphere. LCMS indicated M2 was formed as the major product. Solvent was removed to give the crude mixture. The crude mixture was then purified by silica gel using MeOH/DCM (0-20%) as eluent, providing M2 (300 mg, 149.02 μmol) in 50.00% yield) as expected. MS: [M+CH₃COOH]⁺: 2071.5).

3. Synthesis of intermediate M3. To a solution of M2 in CHCl₃ (10 mL) was added 3-sulfanylpropanoic acid (15.82 mg, 149.02 μmol, 1 eq.) and Et₃N (15.08 mg, 149.02 μmol, 1 eq.). The mixture was then stirred at r.t. for 2 h under N₂ atmosphere. After that, the obtained mixture was used for the next reaction without further operation.

4. Synthesis of intermediate M4. To a mixture of M3 (300 mg, 141.56 μmol, 1 eq.) in DCM (5 mL) was added HOBt (38.25 mg, 283.11 μmol, 2 eq.), EDCI (54.27 mg, 283.11 μmol, 2 eq.), DIEA (73.18 mg, 566.23 μmol, 98.62 μL, 4 eq.) and S2A (59.38 mg, 141.56 μmol, 1 eq.). Then the mixture was stirred overnight at r.t. under N₂ atmosphere. The crude reaction mixture was further purified by silica gel using MeOH/DCM (0-20%) as eluent, providing M4 (140 mg, 55.54 μmol) in 39.2% yield.

5. Synthesis of intermediate M5. To a mixture of M4 (140 mg, 55.54 μmol, 1 eq.), Et3N (252.90 mg, 2.50 mmol, 45 eq.) and DMAP (13.57 mg, 111.08 μmol, 2 eq.) in DCM (5 mL) was added the tetrahydrofuran-2,5-dione (83.37 mg, 833.08 μmol, 15 eq.) at 0° C. Then the mixture was stirred at r.t. overnight under N₂ atmosphere. LCMS indicated M4 was formed as the major product. The mixture was filtered and the solvent was removed under reduced pressure at 40° C., providing the crude product. The crude product was purified by Preparatory HPLC with MeCN/water containing 0.01% HCOONH₄, providing M5 (32 mg, 12.21 μmol, 21.98% yield, 100% purity) as a white solid. MS [M-H]-: 2619.5.

6. Synthesis of intermediate M6. To a mixture of M5 (32 mg, 12.21 μmol, 1 eq.) in MeCN (5 mL) was added DIEA (6.31 mg, 48.84 μmol, 8.51 μL, 4 eq.) and HBTU (9.26 mg, 24.42 μmol , 2 eq.), and the mixture was swirled for 3-4 min followed by addition of CPG-Resin (366.33 mg, 50 μmol/g). The suspension was gently shaken at room temperature on a wrist-action shaker for 48 h then filtered and washed with MeCN (10 mL) twice. The solid was dried under vacuum for 2 h at 45° C. to obtain the white solid (360 mg). To the above white solid (360 mg) pyridine (391.64 mg, 4.95 mmol, 398.42 μL, 40 eq.) in MeCN (5 mL) and acetyl acetate (252.73 mg, 2.48 mmol, 20 eq.) were added at 0° C. The resulting suspension was gently shaken at room temperature (r.t.) on a wrist-action shaker for 30 min then filtered and washed with MeCN (10 ml) twice. The solid was dried under vacuum for 2 h at 45° C., providing M6 (345 mg, 118.62 μmol) as a white solid. Loading: 27.0 umol/g.

7. Synthesis of conjugate 4. Compound IV was obtained from M6 through standard solid-phase oligonucleotide synthesis and de-protection protocols, as described (M. J. Damha, K. K. Ogilvie, Methods Mol. Biol. 1993, 20, 81-114).

### Example 6: Preparation of Conjugate 5

Conjugate 5 was synthesized according to the route of synthesis below:

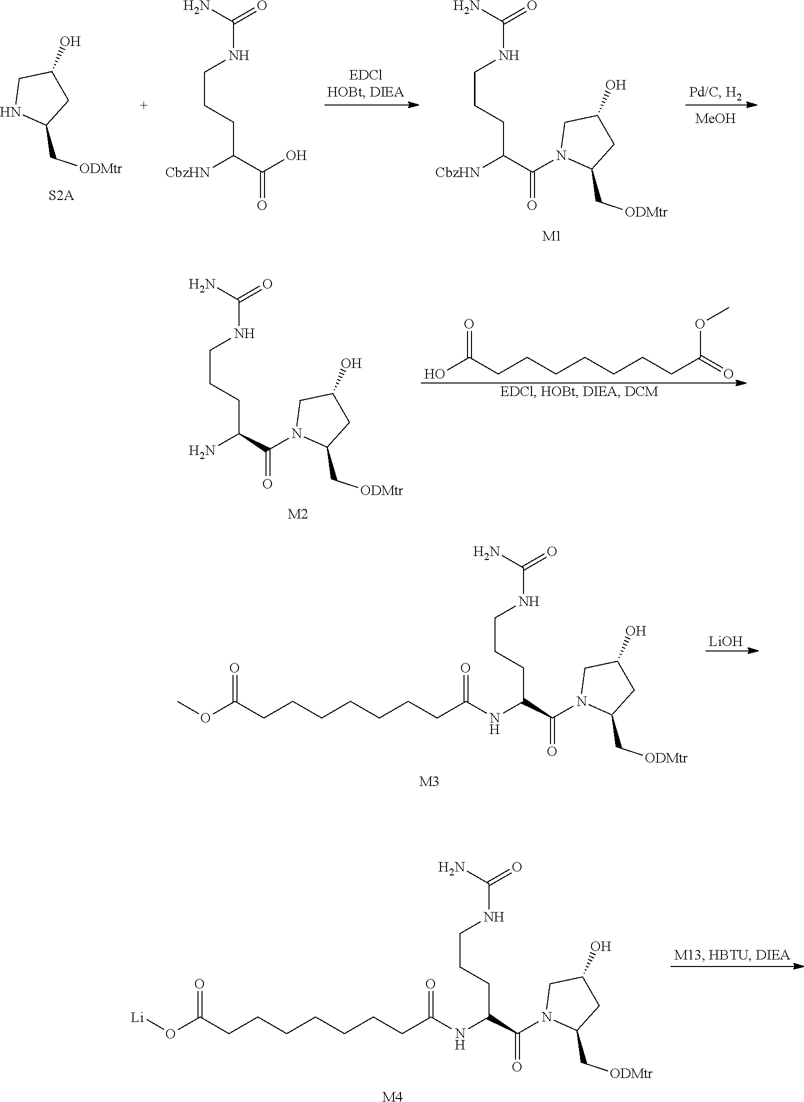

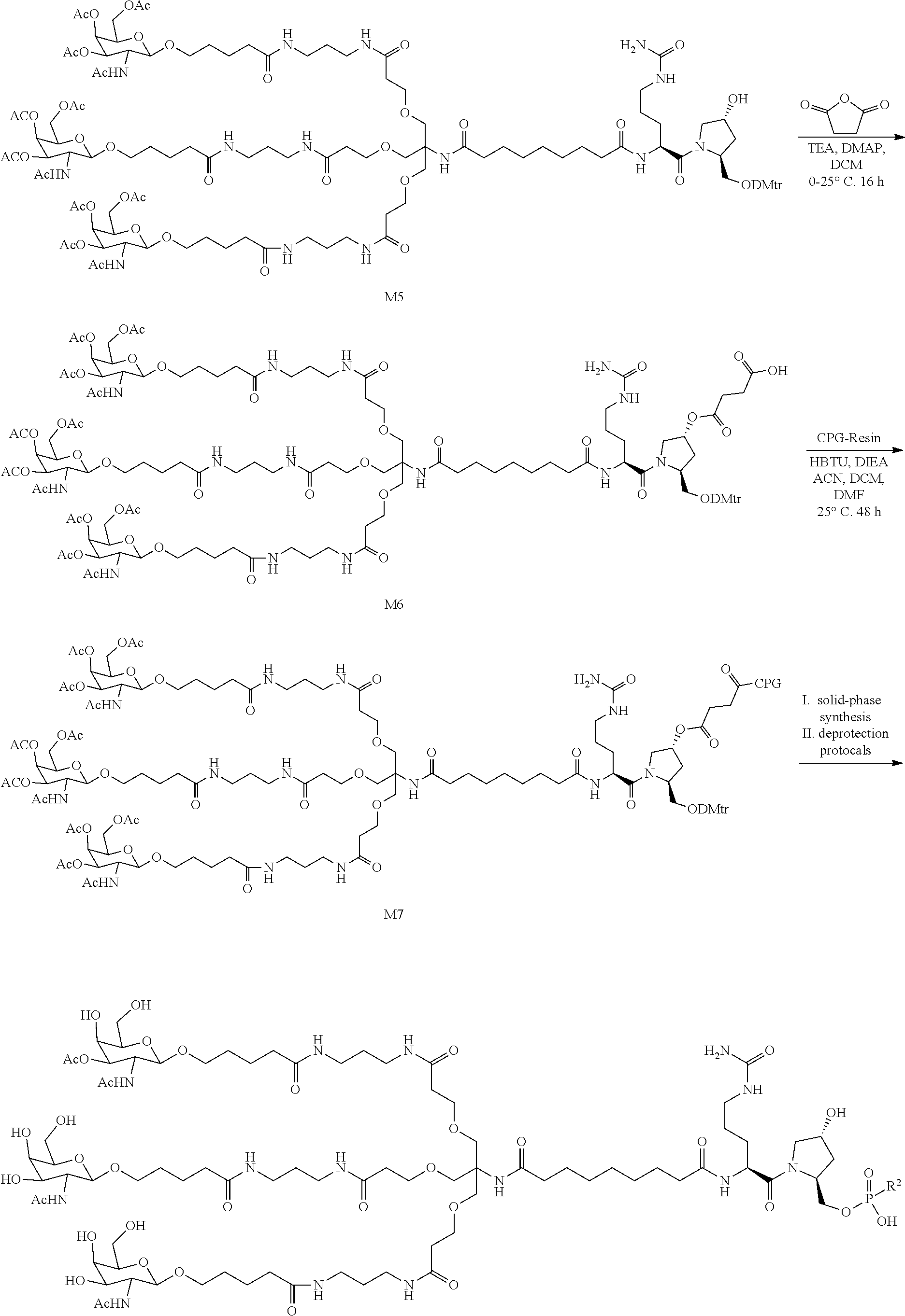

1. Synthesis of intermediate M1. To a mixture of (2S)-2-(benzyloxycarbonylamino)-5-ureido-pentanoic acid (0.8 g, 2.59 mmol, 1 eq.) in DCM (20 mL) was added the EDCI (991.60 mg, 5.17 mmol, 2 eq.), DIEA (1.34 g, 10.35 mmol, 1.80 mL, 4 eq.), HOBt (349.47 mg, 2.59 mmol, 1 eq.) and S2A (1.09 g, 2.60 mmol, 1 eq.). Then the mixture was stirred overnight at r.t. under N₂ atmosphere. LCMS indicated M1 was formed as the major product. The mixture was diluted by DCM (20 mL) and washed with water (20 mL). The organic layer was separated and the collected organic phase was dried with anhydrous Na₂SO₄. Solvent was removed under reduced pressure at 40° C. to give crude product. The crude product was further purified by silica gel (0-3% MeOH/DCM), providing M1 (1.2 g, 1.69 mmol) in 65.0% yield.

2. Synthesis of intermediate M2. To a mixture of M1 (1.2 g, 1.69 mmol, 1 eq.) in MeOH (10 mL) was added Pd/C (102.52 mg, 844.10 μmol , 0.5 eq.). The mixture was stirred overnight at 25° C. under H₂ atmosphere. LCMS indicated M2 was formed as the major product. The mixture was filtered and the solvent was removed under reduced pressure at 40° C. to give M2 (860 mg), which was used for the next step directly.

3. Synthesis of intermediate M3. To a mixture of 9-methoxy-9-oxo-nonanoic acid (301.61 mg, 1.49 mmol, 1 eq.) in DCM (10 mL) was added HBTU (1.13 g, 2.98 mmol, 2 eq.), DIEA (770.94 mg, 5.97 mmol, 1.04 mL, 4 eq.) and M2 (860 mg, 1.49 mmol, 1 eq.). The mixture was then stirred overnight at r.t. under N₂ atmosphere. LCMS indicated M3 was formed as the major product. The mixture was diluted by DCM (20 mL) and then washed with water (20 mL). The organic layer was separated and dried by anhydrous Na₂SO₄. The collected solvent was removed in vacuo at 40° C. to give crude product. The crude product was further purified by silica gel (0-6% MeOH/DCM), providing M3 (680 mg, 893.66 μmol) in 59.93% yield.

4. Synthesis of intermediate M4. To a mixture of methyl M3 (600 mg, 788.53 limo′, 1 eq.) in MeOH (10 mL) and H₂O (10 mL) was added LiOH (188.85 mg, 7.89 mmol, 10 eq.). The mixture was stirred 3 h at 80° C. under N₂ atmosphere. LCMS indicated M3 was consumed. The solvent was removed under reduced pressure at 60° C., providing crude M4 (590 mg), which was used for the next step directly.

5. Synthesis of intermediate M5. To a solution of M4 (201 mg, 267.57 μmol, 1.5 eq.) in DMF (5 mL) was added HOBt (1 eq.), EDCI (2 eq.) and DIEA (2 eq.). The resulting mixture was stirred at 25° C. for 1 h under N₂. M13 (320 mg, 178.38 μmol, 1 eq.) was added and the mixture was stirred at 25° C. for 16 h under Nz. The reaction was monitored by HPLC-MS. To the reaction mixture was added DCM (15 mL) and it was then washed with H₂O (10 mL*3). The organic phase was concentrated for a yellow oil with DMF. The crude product was purified by column chromatography on silica gel with DCM/MeOH (90:10-80:20-70:30) as eluent. M5 (230 mg, 91.17 μmol) was finally obtained.

6. Synthesis of intermediate M6. To a solution of M5 (230 mg, 91.17 μmol, 1 eq.) in DCM (10 mL) was added DMAP (22.28 mg, 182.34 μmol, 2 eq.) and TEA (415.14 mg, 4.10 mmol, 570.25 μL, 45 eq.) and succinic anhydride (136.85 mg, 1.37 mmol, 15 eq.) under an ice bath. The resulting mixture was stirred at 25° C. for 16 h. LC-MS indicated the expected product was formed. The reaction mixture was concentrated under reduced pressure to give the crude product. The crude product was purified by prep-HPLC with MeCN/water containing 0.01% HCOONH₄, providing M6 (33.8 mg, 12.89 μmol) as a white solid with 99.83% HPLC purity. LCMS (ESI): Cal. for C124H184N₁₄0₄₇: 2622.89, Found [M-H]-: 2621.7.

7. Synthesis of intermediate M7. M6 (33.8 mg, 12.89 μmol, 1 eq.) and HBTU (9.77 mg, 25.77 μmol, 2 eq.) were dispersed in MeCN (5 mL). DIEA (6.66 mg, 51.55 μmol, 8.98 μL, 4 eq.) was added to the mixture and the mixture was swirled for 3-4 min followed by addition of CPG-Resin (386.7 mg, 50 μmol/g). The suspension was gently shaken at room temperature on a wrist-action shaker for 48 h. The reaction mixture was filtered and washed with MeCN (25 ml) twice. The solid was dried under vacuum at 35° C. for 2 h, providing the white solid (382.6 mg). Acetyl acetate (1.95 mg, 19.13 μmol, 1.81 μL, 1.46e-1 eq.) and pyridine (4.54 mg, 57.39 μmol, 4.62 μL, 4.37e-1 eq.) were dissolved in ACN (10 mL), and the mixture was swirled for 3-4 min followed by addition of the above white solid (382.60 mg). The suspension was gently shaken at room temperature on a wrist-action shaker for 30 min and then filtered, and washed with MeCN (10 ml) twice. The solid was dried under vacuum for 2 h at 40° C., providing M7 (261.1 mg) as a white solid. Loading: 20.5 umol/g.

8. Synthesis of conjugate 5. Conjugate 5 was obtained from M7 through standard solid-phase oligonucleotide synthesis and de-protection protocols, as described (M. J. Damha, K. K. Ogilvie, Methods Mol. Biol. 1993, 20, 81-114).

### Example 7: Conjugate Bioassays in Primary Hepatocytes and Humanized PCSK9 Mice

Free uptake in primary hepatocytes of cynomolgus monkey. The primary liver cells of cynomolgus monkey (cryopreserved) were obtained from MiaoTong (Shanghai) Biotechnology Co., Ltd. The cells were cultured in the resuscitation medium in a humidified incubator in the atmosphere of 37° C. and 5% CO₂. After resuscitation, hepatocytes were seeded into 96 well plates coated with medium at a density of 5×10⁵ cells/well. After 24 hours of adherence, the supernatant was drawn, and test conjugate (siRNA) was added (starting at 500 nm, 5 times dilution, 3 times in total) and maintenance medium was added for culture.

After co-culturing for 48 hours, the primary hepatocytes were lysed, and mRNA was extracted using Dynabeads' mRNA Purification Kit according to the experimental scheme. cDNA was obtained by reverse transcription, and the mRNA levels of PCSK9 and GAPDH were detected by the SYBR green method. A standardized PCSK9/GAPDH ratio was used as the relative level of PCSK9 mRNA.

Use of Humanized PCSK9 mice to determine PCSK9 knockdown level. 42 PCSK9 humanized male mice (from Shanghai Model Organisms Center) were randomly divided into seven groups (n=6). Group A was subcutaneously injected with conjugate 1, which was diluted with normal saline to a designated dose (9 mg/kg); Group B received subcutaneous injection of conjugate 2 diluted with normal saline to the designated dose (9 mg/kg); Group C received subcutaneous injection of conjugate 3 diluted with normal saline to the designated dose (9 mg/kg); Group D was subcutaneously injected with conjugate 4 diluted with normal saline to the designated dose (9 mg/kg); Group E was subcutaneously injected with conjugate 5 diluted with normal saline to the designated dose (9 mg/kg); Group I was the blank control group which was subcutaneously injected with the same volume of normal saline; and Group II was the positive control group which was subcutaneously injected with the designated dose (9 mg/kg) of Inclisiran as positive control. According to the corresponding time point, 100 μl blood samples were collected from orbit. After anticoagulation with EDTA, plasma was obtained by centrifugation and frozen at −80° C. At the end of the experiment, ELISA or biochemical analysis was used to detect the serum humanized PCSK9 protein level and blood lipid levels.

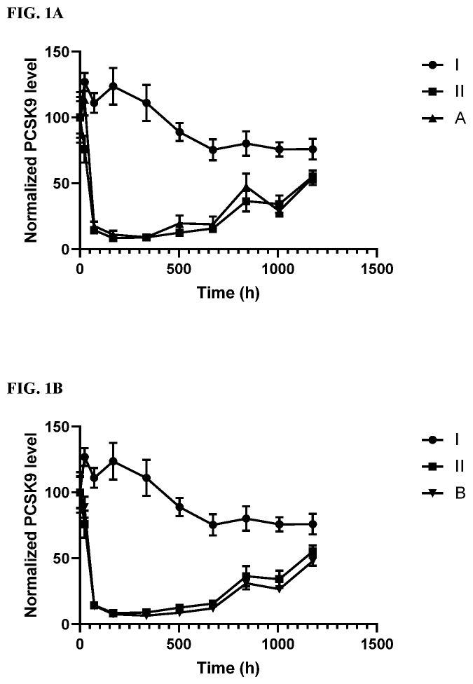

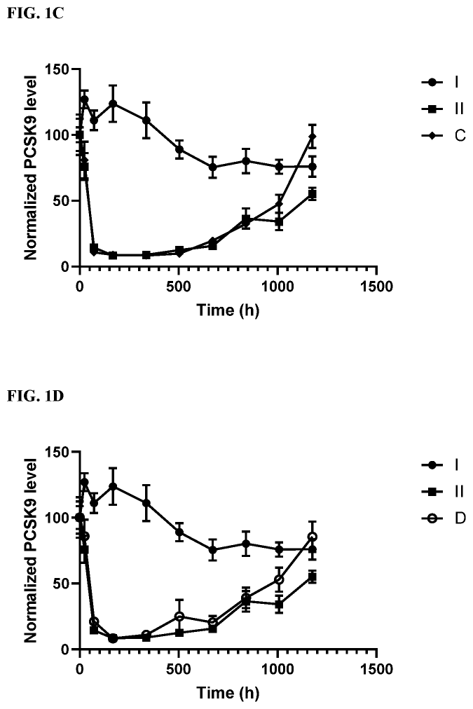

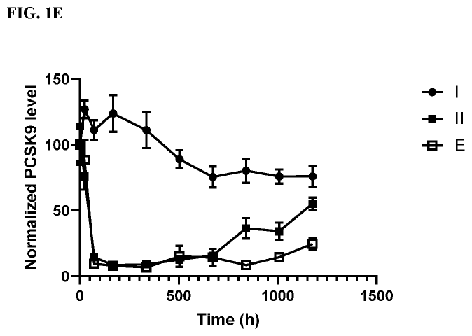

Detection of PCSK9 by ELISA. Humanized PCSK9 protein levels were detected by ELISA according to standard experimental protocol (provided by the supplier (Shanghai Model Organisms Center)). After the sample was fully dissolved, it was diluted 10 times with PBS and then added into the ELISA plate coated with capture antibody. After incubation at room temperature for 2 hours, the plate was washed and incubated with biotinylated detection antibody and SA-HRP mixture at room temperature for 1 hour. After cleaning, TMB was used to develop the color, and a SpectraMax® M5e multi-function enzyme reader was used to detect 450 nm light absorption. The standard curve was fitted with four parameters and used for the conversion of humanized PCSK9 protein concentration. Results are shown in FIGS. 1A-1E. Notably, for the conjugate 5 treated group, the Humanized PCSK9 protein level was similar to that of the positive control group during the early stages of the experiment, and significantly lower than that of the positive control group in the later stages of the experiment (days 35, 42, and 49)(FIG. 1E). For the conjugate 2 treated group, the mice showed lower level of PCSK9 protein at all timepoints (FIG. 1B).

Detection of blood lipid levels. Animal blood samples were collected on day 3, 7, 14, 21, 28, 35, 42, and 49. After the serum samples were fully dissolved, the same volume of normal saline was added for dilution, and the levels of high-density lipoprotein cholesterol (HDL-C), low-density lipoprotein cholesterol (LDL-C), total cholesterol (TC) and total triglyceride (TG) were detected by using the corresponding analytical kit. The Chemray™ 800 produced by Shenzhen DuLei Life Science and Technology Co., Ltd. was used as an automatic biochemical analyzer. All tests were carried out by Shanghai BiYuntian Biotechnology Co., Ltd.

For all treatment groups, the results showed reduction of total cholesterol level in comparison to the control group. For the group treated with conjugate 3, total cholesterol level was significantly lower in comparison to the positive control group on days 3, 7, 14, 21, 28, and 35. For the conjugate 5 treated group, the total cholesterol level was similar to that of the positive control group during the early stages of the experiment, and significantly lower than that of the positive control group in the later stages of the experiment (days 35, 42, and 49).

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

The contents of all documents and references cited herein are hereby incorporated by reference in their entirety.

What is claimed is:

1. A bivalent compound, or a pharmaceutically acceptable salt or ester thereof, capable of being conjugated directly or indirectly to both a targeting moiety and a bioactive agent to form a corresponding conjugate, wherein the bivalent compound is a compound of Formula (I): 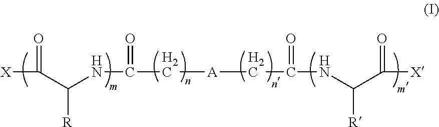
wherein:
X and X′ are independently selected from a hydroxyl group (OH), an amino group (NH₂), and a halide;
R and R′ are independently a side chain of a natural or unnatural amino acid, and R and R′ may be the same or different;
m and m′ are independently an integer from 0 to 3,
provided that when m or m′ is 1, one or both of the structural fragment 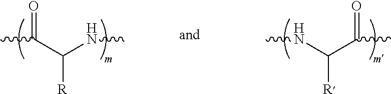
is a natural or unnatural amino acid residue and the structural fragments may be the same or different;
provided that when m or m′ is 2 or 3, one or both of the structural fragment 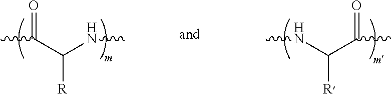
is a residue of a dipeptide or tripeptide and the structural fragments may comprise the same or different amino acid residues;
n and n′ are independently an integer from 0 to 10, provided that n and n′ are not both 0; and
A is selected from 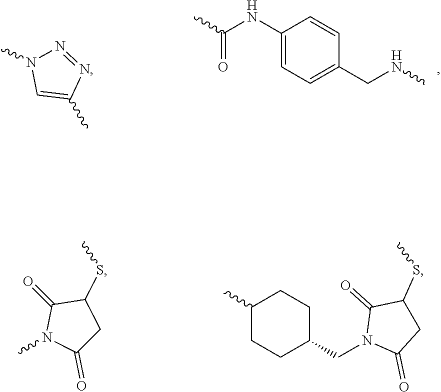
and a methylene (CH₂), provided when A is a methylene, m and m′ are not both 0.

2. The bivalent compound of claim 1, wherein A is methylene and the compound is a compound of Formula (II):

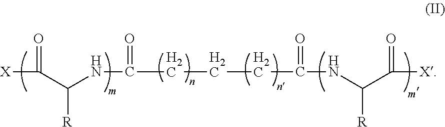

3. The bivalent compound of claim 2, wherein n is 6, n′ is 0, and at least one of m and m′ is not 0.

4. The bivalent compound of any one of claims 1 to 3, wherein the amino acid is independently selected from citrulline, homocitrulline, lysine, homolysine, asparagine, glutamine, arginine, glycine, methionine, phenylalanine, albizziin, valine, and combinations thereof.

5. The bivalent compound of any one of claims 1 to 4, wherein the compound is a compound of Formula (III) or Formula (IV): 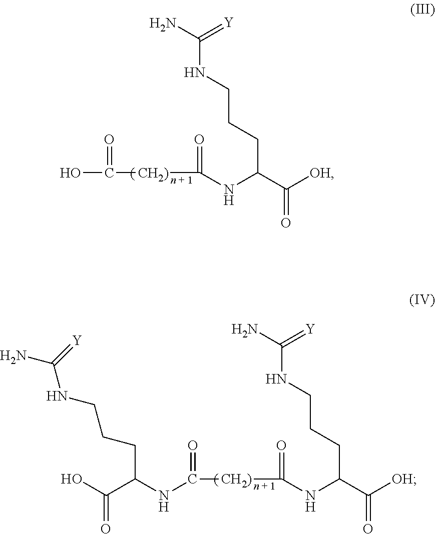
where Y is selected from oxygen (O) and nitrogen (NH).

6. The bivalent compound of any one of claims 1 to 5, wherein the compound is: 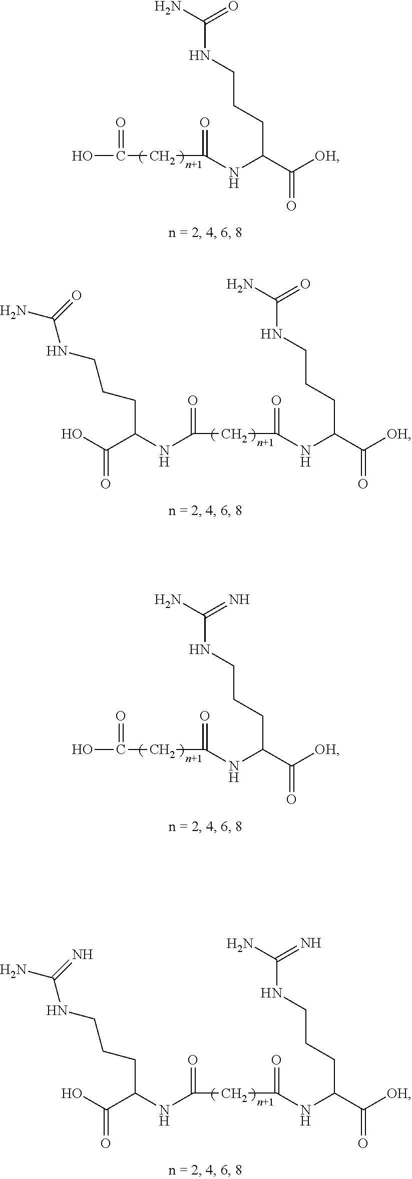 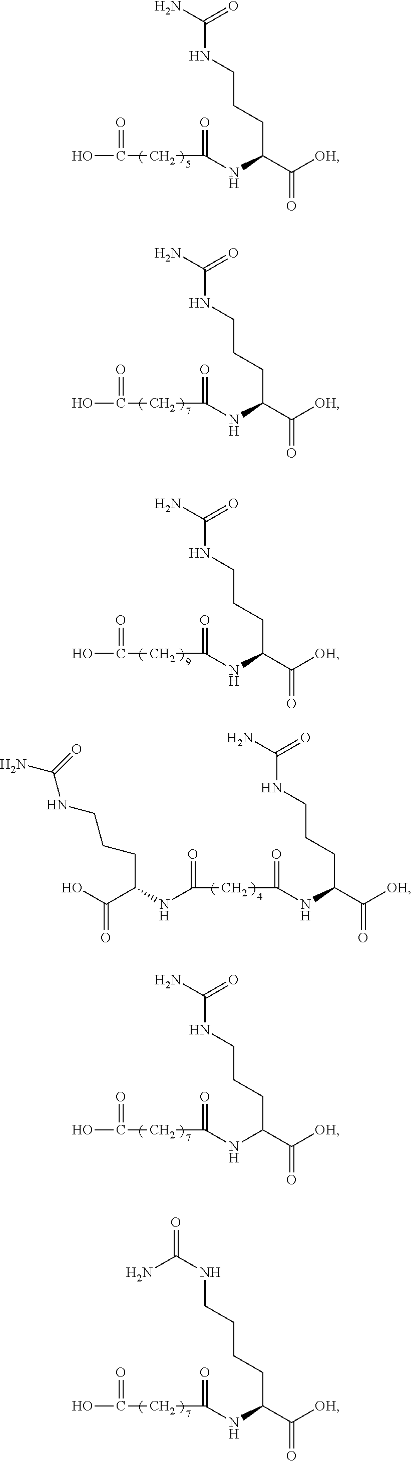 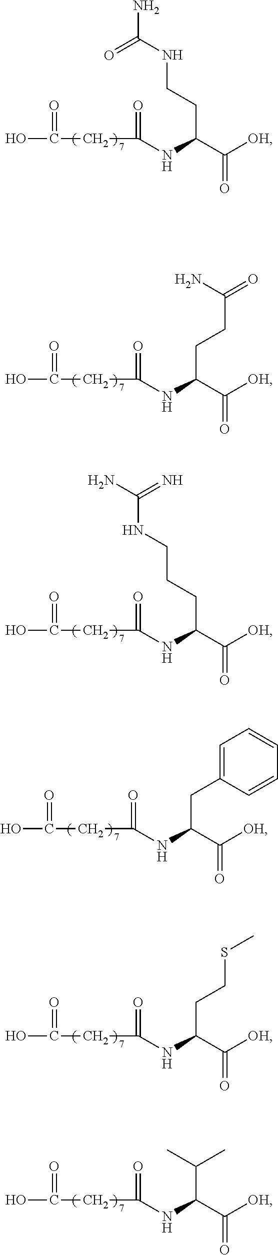 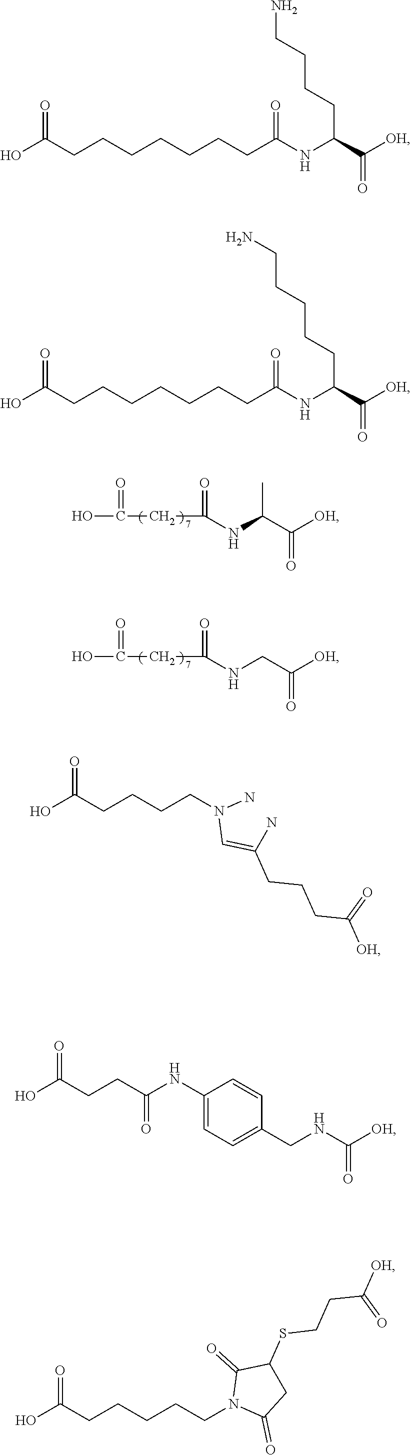 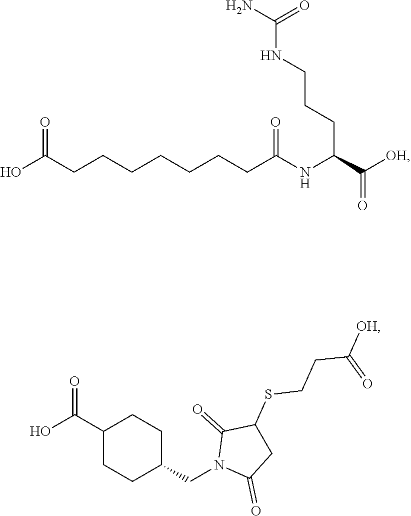
or a pharmaceutically acceptable salt or ester thereof.

7. The bivalent compound of claim 6, wherein the compound is: 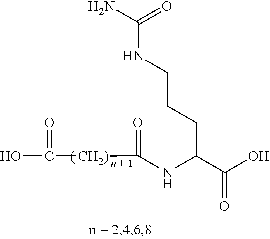
or a pharmaceutically acceptable salt or ester thereof.

8. The bivalent compound of claim 6, wherein the compound is: 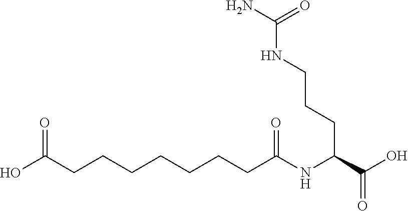 [SMILES: NC(=O)NCCC[C@H](NC(=O)CCCCCCCC(=O)O)C(=O)O]
or a pharmaceutically acceptable salt or ester thereof.

9. A conjugate comprising the bivalent compound of any one of claims 1 to 8, wherein the conjugate has the structure of Formula (V), or is a pharmaceutically acceptable salt or ester thereof: 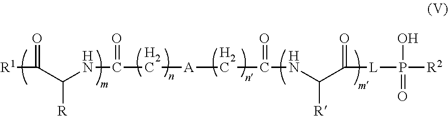
wherein:
R¹ is a targeting moiety comprising one or more carbohydrate, polypeptide and/or lipophile;
R² is a bioactive agent; and
L is a linker moiety comprising a nitrogen-containing heterocyclic ring;
the bivalent compound being conjugated to R¹ directly and to R² indirectly through the fragment —L—P(O)(OH)—, to form the conjugate.

10. The conjugate of claim 9, wherein the linker moiety comprises a 4-membered-, a 5-membered-, or a 6-membered-heterocyclic ring.

11. The conjugate of claim 9 or 10, wherein L is selected from one of the following heterocyclic systems: 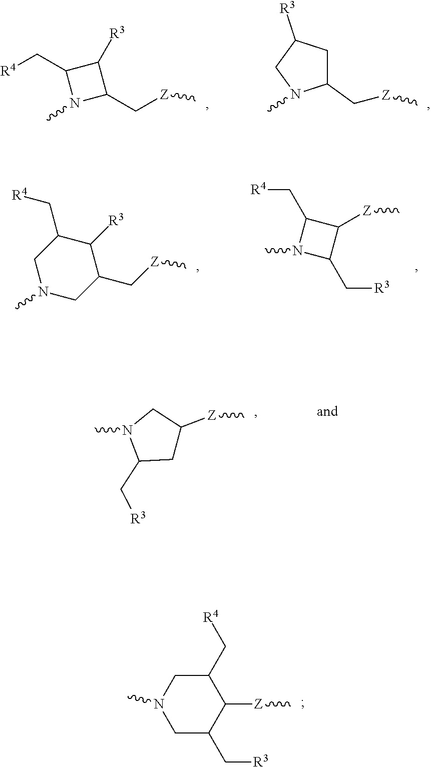
where:
Z is selected from oxygen (O), sulfur (S), and nitrogen (NH); and
R³ and R⁴ are independently selected from H, OH, and OR⁵, wherein R⁵ is a protecting group or a substituent for hydroxyl selected from alky, aryl, alkylaryl, acyl, and phosphonyl group.

12. The conjugate of any one of claims 9 to 11, wherein the bioactive agent is selected from an antibody or an antigen-binding fragment thereof, an oligonucleotide, a hormone, and an antibiotic.

13. The conjugate of claim 12, wherein the bioactive agent is an oligonucleotide.

14. The conjugate of claim 12 or 13, wherein the oligonucleotide is selected from an siRNA, a miRNA, an anti-microRNA, a microRNA antagonist, a microRNA mimic, a decoy oligonucleotide, an immunostimulator, a guanine (G) quadruplex DNA or RNA, a guanine (G) tetraplex DNA or RNA, an alternative splice, a single-stranded RNA, a double stranded RNA, an antisense nucleic acid, an aptamer, a stem-loop RNA, an mRNA fragment, an activating RNA and an activating DNA.

15. The conjugate of any one of claims 12 to 14, wherein the oligonucleotide is an siRNA, optionally a double stranded siRNA, and wherein each nucleotide residue in the siRNA is, independently, modified or unmodified.

16. The conjugate of claim 15, wherein the oligonucleotide is a double stranded siRNA.

17. The conjugate of claim 16, wherein the siRNA comprises or consists of the sequence set forth in any one of SEQ ID NOs. 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, 55/56, 57/58, 59/60, 61/62, 63/64, 65/66, 67/68, 69/70, 71/72, 73/74, 75/76, 77/78, 79/80, 81/82, 83/84, 85/86, 87/88, 89/90, 91/92, 93/94, 95/96, 97/98, 99/100, 101/102, 103/104, 105/106, 107/108, 109/110, 111/112, 113/114, 115/116, 117/118, 119/120, 121/122, 123/124, 125/126, 127/128, 129/130, 131/132, 133/134, 135/136, 137/138, 139/140, 141/142, 143/144, 145/146, 147/148, 149/150, 151/152, 153/154, 155/156, 157/158, 159/160, 161/162, 163/164, 165/166, 167/168, 169/170, 171/172, 173/174, 175/176, 177/178, 179/180, 181/182, 183/184, 185/186, 187/188, 189/190, 191/192, 193/194, 195/196, 197/198, 199/200, 201/202, 203/204, 205/206, 207/208, 209/210, 211/212, 213/214, 215/216, 217/218, 219/220, 221/222, 223/224, 225/226, 227/228, 229/230, 231/232, 233/234, 235/236, and 237/238; or a sequence listed in Table 2 or Table 3.

18. The conjugate of claim 15 or 16, wherein the siRNA comprises of consists of the sequence set forth in SEQ ID Nos: 5/6, as follows:
the sense strand comprises or consists of the nucleotide sequence of:
(SEQ ID NO: 5)
5′-GmCmCmUGGAGmUmUmUAmUmUmCGGAAdTdT-3′
and
the antisense strand comprises or consists of the nucleotide sequence of:
(SEQ ID NO: 6)
5′-PmUUfmCCfmGAfmAUfmAAfmACfmUCfmCAfmGGfmCdTdT-3′.

19. The conjugate of claim 15 or 16, wherein the siRNA comprises of consists of the sequence set forth in SEQ ID Nos: 1/2, as follows:
the sense strand comprises or consists of the nucleotide sequence of:
(SEQ ID NO: 1)
5′-mC*mU*mAmGmAmCCfmUGfmUdTmUmUmGmCmUmUmUmUmGmU-3′
and
the antisense strand comprises or consists of the nucleotide sequence of:
(SEQ ID NO: 2)
5′-mA*Cf*mAAfAfAfmGCfmAAfmAAfmCAfmGGfmUCfmUmAmG*m
A*mA-3′.

20. The conjugate of any one of claims 14 to 19, wherein the target of said siRNA is selected from ApoB, ApoC, ANGPTL3, PCSK9, SCD1, FVII, p53, HBV, and HCV.

21. The conjugate of any one of claims 12 to 20, wherein said siRNA is PCSK9-siRNA.

22. The conjugate of claim 21, wherein the PCSK9-siRNA has the following sequence:
Sense strand:
(SEQ ID NO: 147)
5′-mC*mUmAmGmAmCCfmUGfmUdTmUmUmGmCmUmUmUmUmGmU-3′,
and
Antisense strand:
(SEQ ID NO: 148)
5′-mA*Cf*mAAfAfAfmGCfmAAfmAAfmCAfmGGfmUCfmUmAmG*m
A*mA-3′;
where:
C, G, U, and A represent cytidine-3′-phosphate, guanosine-3′-phosphate, uridine-3′-phosphate, and adenosine-3′-phosphate respectively;
m means that the adjacent nucleotide on the right side of the letter m is a 2′-O-methyl-modified nucleotide;
f means that the adjacent nucleotide on the left side of the letter f is a 2′-fluoro-modified nucleotide;
indicates that the adjacent nucleotide on the left side of * is a thiophosphate-modified nucleotide;
f* indicates that the adjacent nucleotide on the left side of f* is a nucleotide modified by thiophosphate and 2′-fluorine at the same time;
d means that the adjacent nucleotide to the right of the letter d is a 2′-deoxyribonucleotide; and
T represents 5′-methyluridine-3′-phosphate.

23. The conjugate of any one of claims 12 to 20, wherein said siRNA is ANGPTL3-siRNA.

24. The conjugate of claim 23, wherein the ANGPTL3-siRNA has the following sequence:
Sense strand:
(SEQ ID NO: 179)
5′-mAmAmAmUmCmAmCGfmAAfdAmCmCAfmAmCmUmAmU-3′,
and
Antisense strand:
(SEQ ID NO: 180)
5′-mAUfmAmGmUmUmGGfmUUfmUCfmGUfmGmAmUmUmUCfmC-3′;
where:
C, G, U, and A represent cytidine-3′-phosphate, guanosine-3′-phosphate, uridine-3′-phosphate, and adenosine-3′-phosphate respectively;
m means that the adjacent nucleotide on the right side of the letter m is a 2′-O-methyl-modified nucleotide;
f means that the adjacent nucleotide on the left side of the letter f is a 2′-fluoro-modified nucleotide;
indicates that the adjacent nucleotide on the left side of * is a thiophosphate-modified nucleotide;
f* indicates that the adjacent nucleotide on the left side of f* is a nucleotide modified by thiophosphate and 2′-fluorine at the same time; and
d means that the adjacent nucleotide to the right of the letter d is a 2′-deoxyribonucleotide.

25. The conjugate of any one of claims 9 to 24, wherein the bivalent compound is connected, directly or indirectly, to the 3′- or 5′-terminal position of an siRNA.

26. The conjugate of any one of claims 9 to 25, wherein R¹ is a lipophile.

27. The conjugate of any one of claims 9 to 26, wherein the lipophile is selected from cholesteryl, cholic acid, adamantane acetic acid, 1-pyrenebutyric acid, dihydrotestosterone, 1,3-bis-o-(hexadecyl)glycerol, geranyloxyhexyl, hexadecyl glycerol, borneol, menthol, 1,3-propanediol, heptadecyl, palmitic acid, myristic acid, O-3-(oleoyl) lithocholic acid O-3-(oleoyl)cholelenic acid, dimethoxy tribenzyl, phenoxazine, and combinations thereof.

28. The conjugate of any one of claims 9 to 25 and 27, wherein R¹ is a carbohydrate.

29. The conjugate of any one of claims 9 to 28, wherein the carbohydrate is selected from alose, atrose, arabinose, cladose, erythritose, erythritonose, fructose, d-fucitol, l-fucitol, fucosamine, fucose, fucose, fucose, galactosamine, galactosamine, n-acetyl-galactosamine (GalNAc), galactose, glucosamine, N-acetyl-glucosamine, glucosamine, glucose, glucose-6-phosphate, gululose glyceraldehyde, l-glycerol-d-mannose-heptose, glycerol, glycerone, gululose, idulose, lysulose, mannosamine, mannose, mannose-6-phosphate, aloulose, quinulose, quinofuramine, rhamnol, rhamnosamine, rhamnose, ribose, ketulose, sedum heptanulose, sorbose, tagatose, talose tartaric acid, threose, xylose, and combinations thereof.

30. The conjugate of any one of claims 9 to 29, wherein the targeting moiety comprises N-acetyl galactosamine (GalNAc).

31. The conjugate of any one of claims 9 to 30, wherein the targeting moiety binds to a cell-surface receptor.

32. The conjugate of claim 31, wherein the cell-surface receptor is an asialoglycoprotein cell receptor (ASGPR).

33. The conjugate of any one of claims 9 to 32, wherein R¹ is selected from:

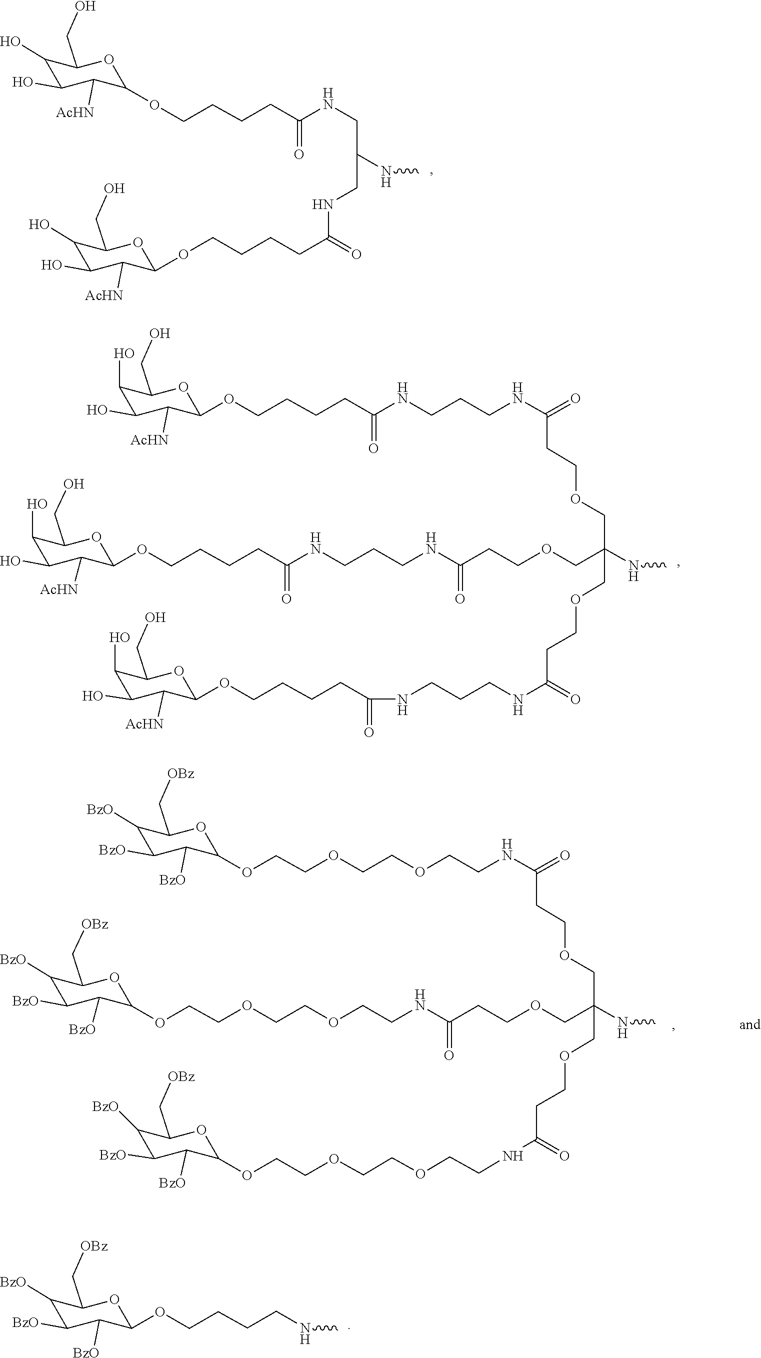

34. The conjugate of any one of claims 9 to 33, wherein the conjugate is: 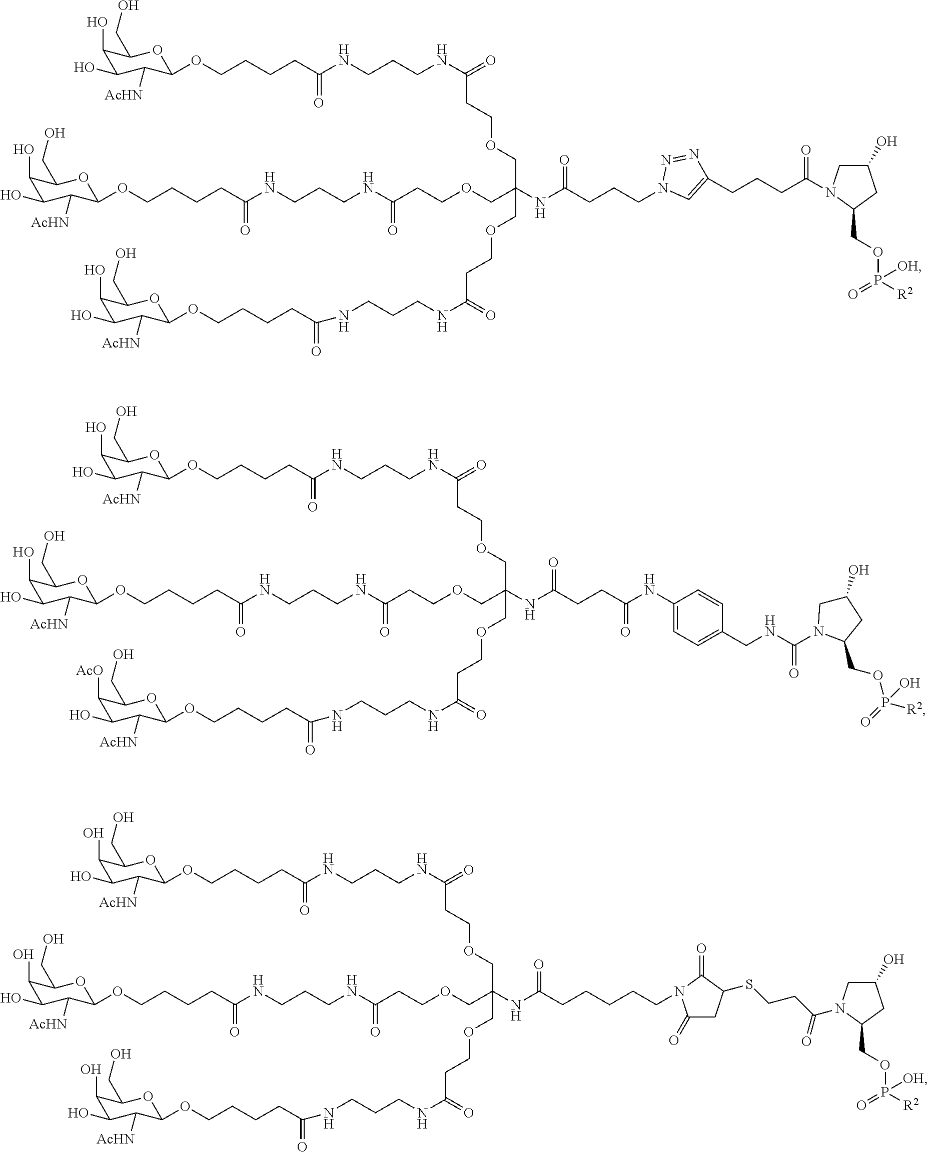 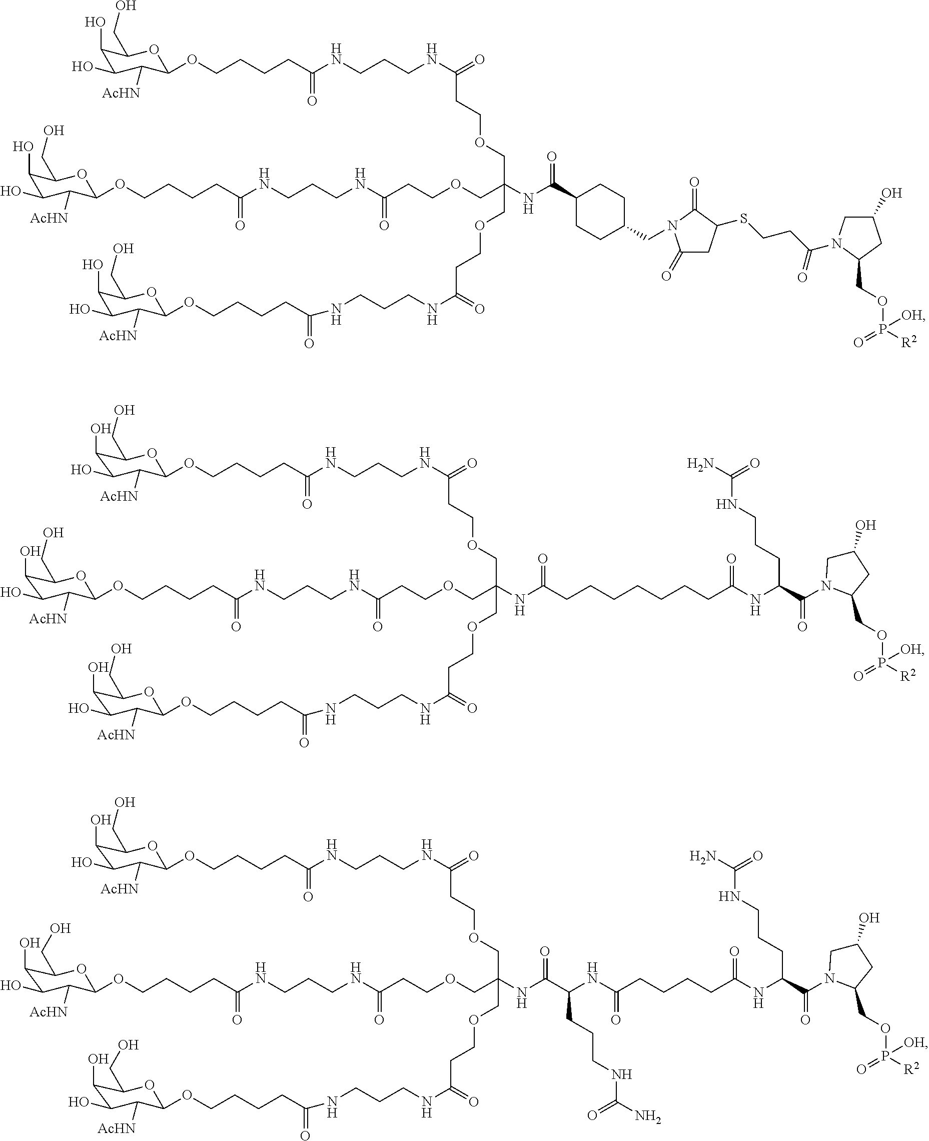 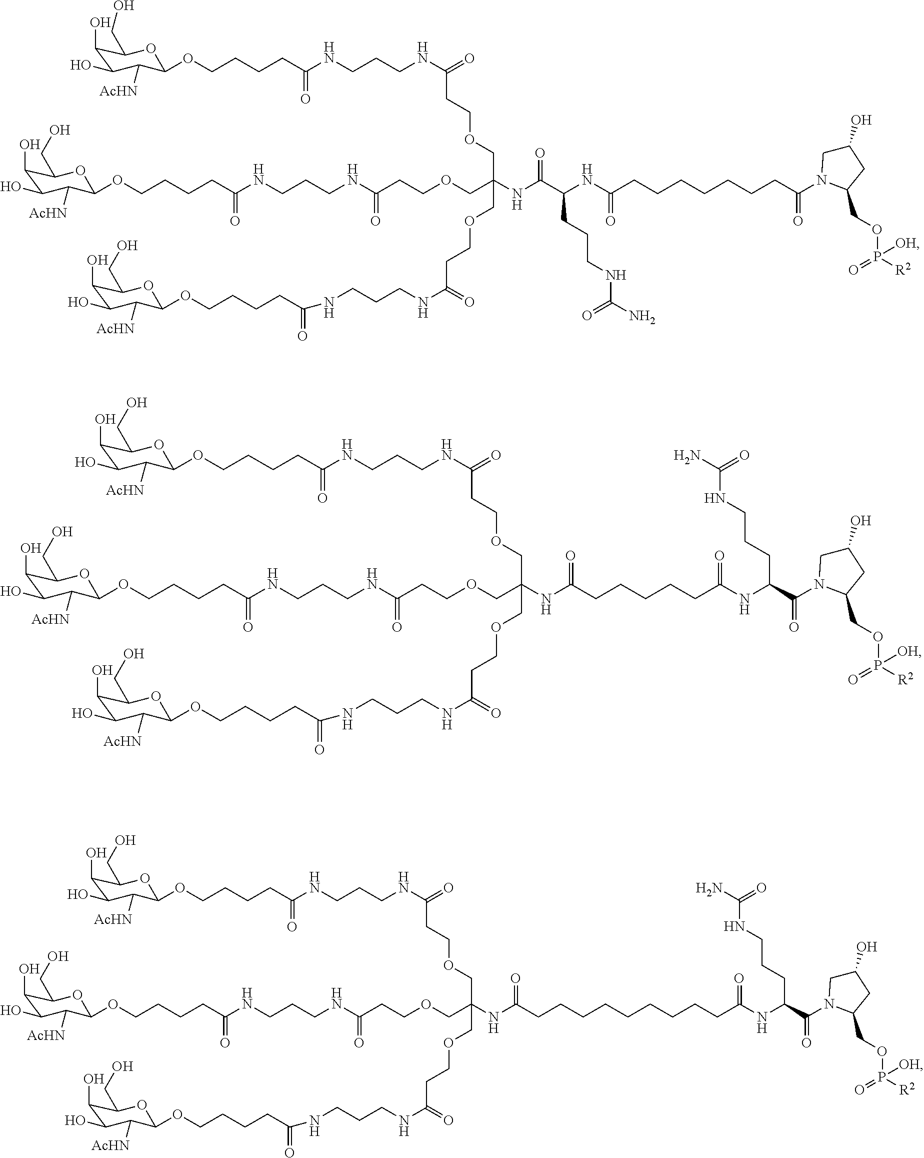 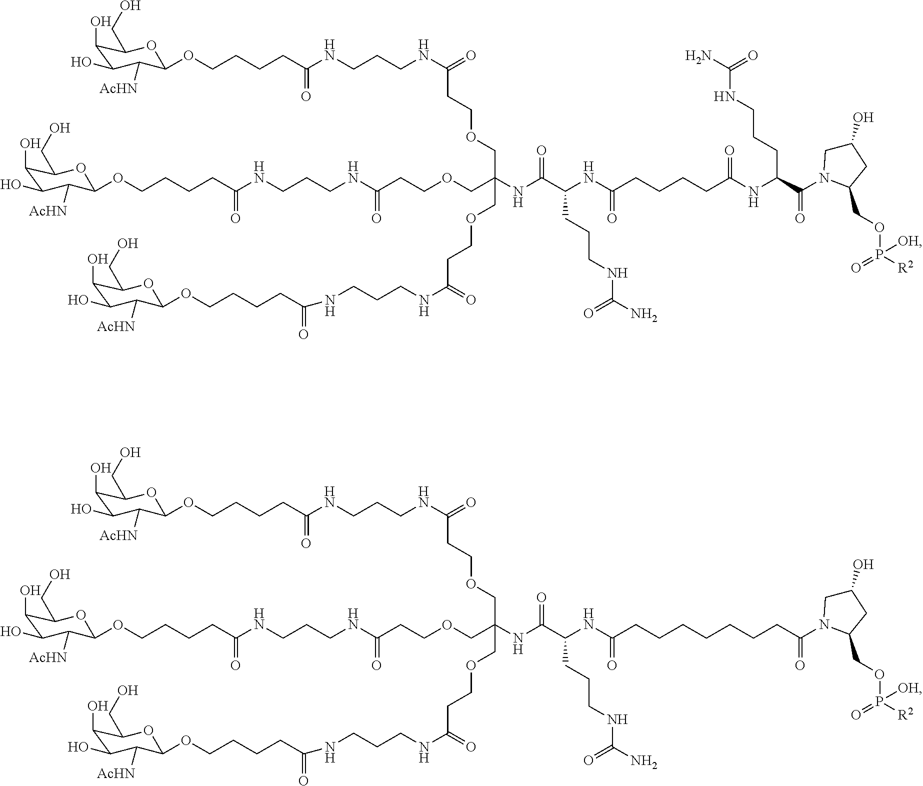 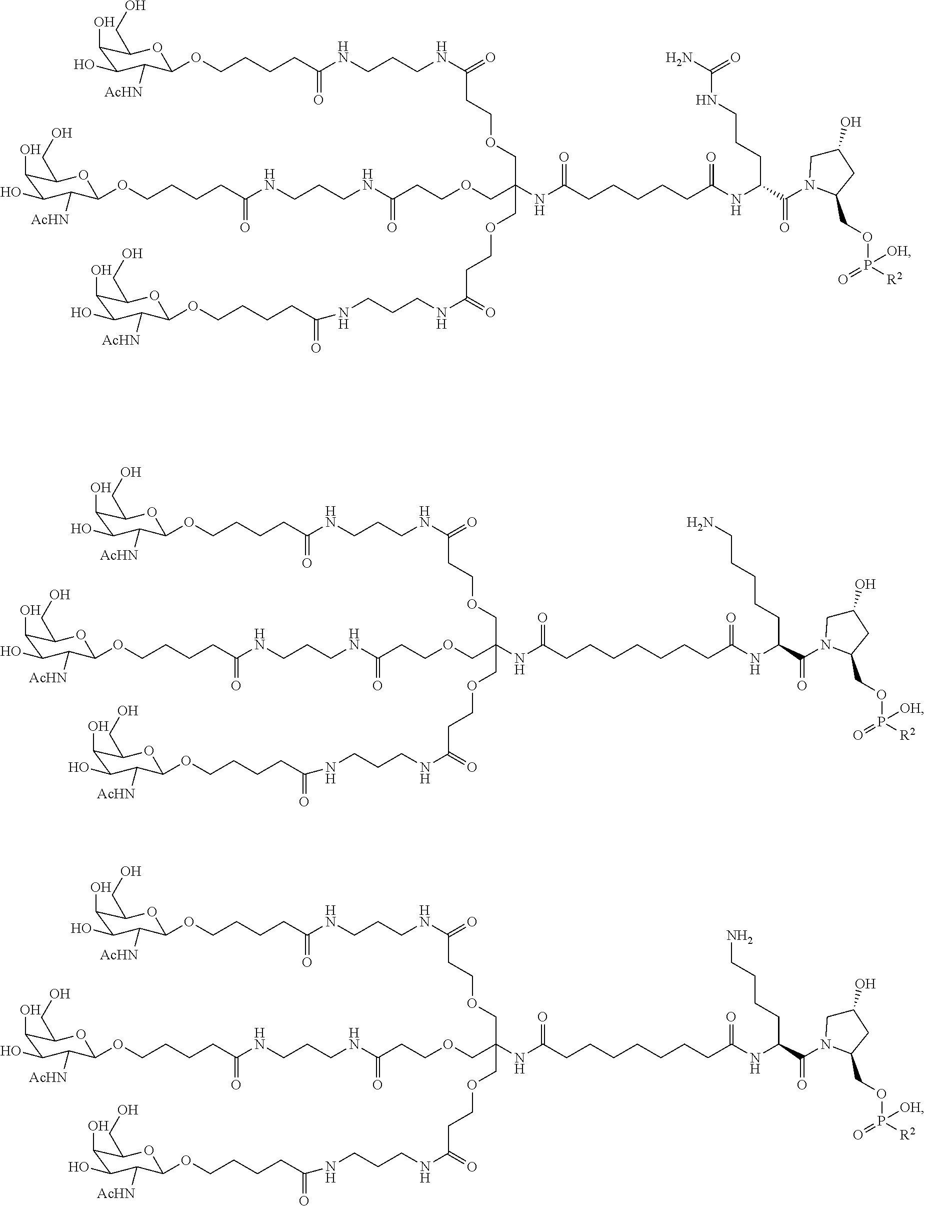 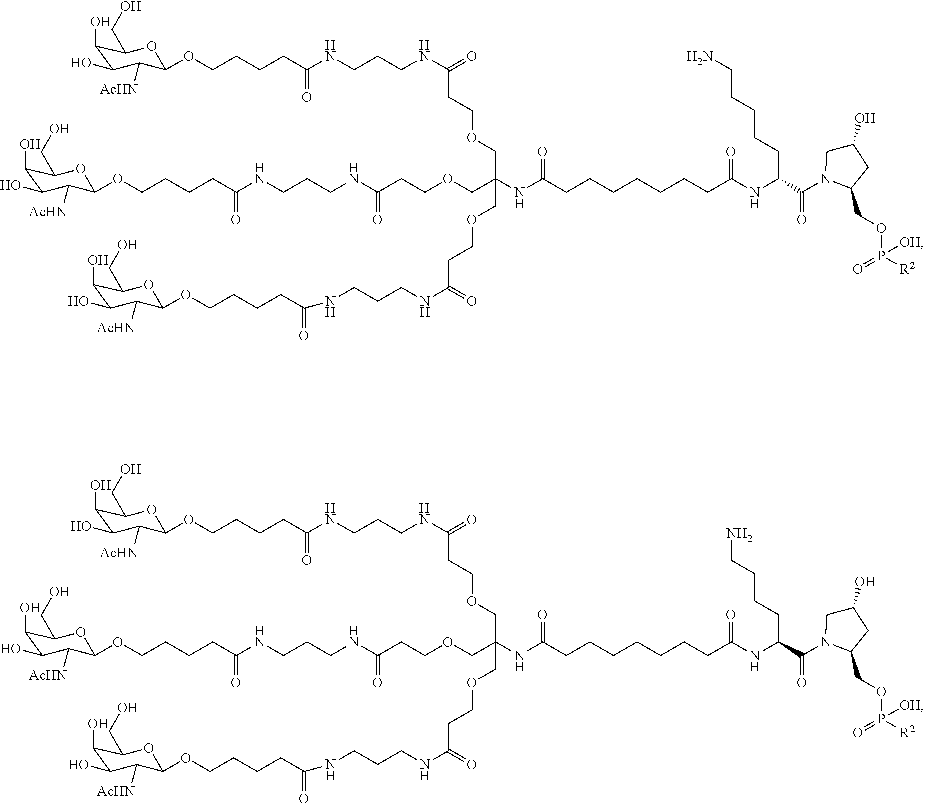
or a pharmaceutically acceptable salt or ester thereof.

35. The conjugate of any one of claims 9 to 34, wherein R² is an oligonucleotide having the following sequence:
Sense strand:
(SEQ ID NO: 147)
5′-mC*mUmAmGmAmCCfmUGfmUdTmUmUmGmCmUmUmUmUmGmU-3′,
and
Antisense strand:
(SEQ ID NO: 148)
5′-mA*Cf*mAAfAfAfmGCfmAAfmAAfmCAfmGGfmUCfmUmAmG*
mA*mA-3′;
where:
C, G, U, and A represent cytidine-3′-phosphate, guanosine-3′-phosphate, uridine-3′-phosphate, and adenosine-3′-phosphate respectively;
m means that the adjacent nucleotide on the right side of the letter m is a 2′-O-methyl-modified nucleotide;
f means that the adjacent nucleotide on the left side of the letter f is a 2′-fluoro-modified nucleotide;
indicates that the adjacent nucleotide on the left side of * is a thiophosphate-modified nucleotide;
f* indicates that the adjacent nucleotide on the left side of f* is a nucleotide modified by thiophosphate and 2′-fluorine at the same time;
d means that the adjacent nucleotide to the right of the letter d is a 2′-deoxyribonucleotide; and
T represents 5′-methyluridine-3′-phosphate.

36. The conjugate of any one of claims 9 to 34, wherein R² is an oligonucleotide having the following sequence:
Sense strand:
(SEQ ID NO: 179)
5′-mAmAmAmUmCmAmCGfmAAfdAmCmCAfmAmCmUmAmU-3′,
and
Antisense strand:
(SEQ ID NO: 180)
5′-mAUfmAmGmUmUmGGfmUUfmUCfmGUfmGmAmUmUmUCfmC-3′,
where:
C, G, U, and A represent cytidine-3′-phosphate, guanosine-3′-phosphate, uridine-3′-phosphate, and adenosine-3′-phosphate respectively;
m means that the adjacent nucleotide on the right side of the letter m is a 2′-O-methyl-modified nucleotide;
f means that the adjacent nucleotide on the left side of the letter f is a 2′-fluoro-modified nucleotide;
indicates that the adjacent nucleotide on the left side of * is a thiophosphate-modified nucleotide;
f* indicates that the adjacent nucleotide on the left side of f* is a nucleotide modified by thiophosphate and 2′-fluorine at the same time; and
d means that the adjacent nucleotide to the right of the letter d is a 2′-deoxyribonucleotide.

37. A conjugate according to any one of claims 1 to 36, wherein the conjugate comprises a compound listed in Table 1, or a pharmaceutically acceptable salt or ester thereof.

38. A conjugate according to claim 37, wherein the conjugate comprises an oligonucleotide having the sequence set forth in any one of SEQ ID NOs. 1/2, 3/4, 5/6, 7/8, 9/10, 11/12, 13/14, 15/16, 17/18, 19/20, 21/22, 23/24, 25/26, 27/28, 29/30, 31/32, 33/34, 35/36, 37/38, 39/40, 41/42, 43/44, 45/46, 47/48, 49/50, 51/52, 53/54, 55/56, 57/58, 59/60, 61/62, 63/64, 65/66, 67/68, 69/70, 71/72, 73/74, 75/76, 77/78, 79/80, 81/82, 83/84, 85/86, 87/88, 89/90, 91/92, 93/94, 95/96, 97/98, 99/100, 101/102, 103/104, 105/106, 107/108, 109/110, 111/112, 113/114, 115/116, 117/118, 119/120, 121/122, 123/124, 125/126, 127/128, 129/130, 131/132, 133/134, 135/136, 137/138, 139/140, 141/142, 143/144, 145/146, 147/148, 149/150, 151/152, 153/154, 155/156, 157/158, 159/160, 161/162, 163/164, 165/166, 167/168, 169/170, 171/172, 173/174, 175/176, 177/178, 179/180, 181/182, 183/184, 185/186, 187/188, 189/190, 191/192, 193/194, 195/196, 197/198, 199/200, 201/202, 203/204, 205/206, 207/208, 209/210, 211/212, 213/214, 215/216, 217/218, 219/220, 221/222, 223/224, 225/226, 227/228, 229/230, 231/232, 233/234, 235/236, and 237/238; or a sequence listed in Table 2 or Table 3.

39. The conjugate according to claim 38, wherein the conjugate comprises: 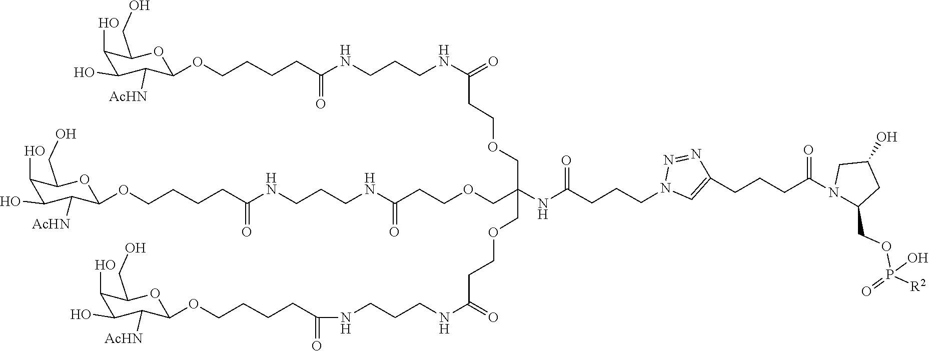
wherein R² is an oligonucleotide comprising or consisting of the sequence set forth in SEQ ID NO: 1/2, 5/6, 147/148, or 179/180;
or a pharmaceutically acceptable salt or ester thereof.

40. The conjugate according to claim 38, wherein the conjugate comprises: 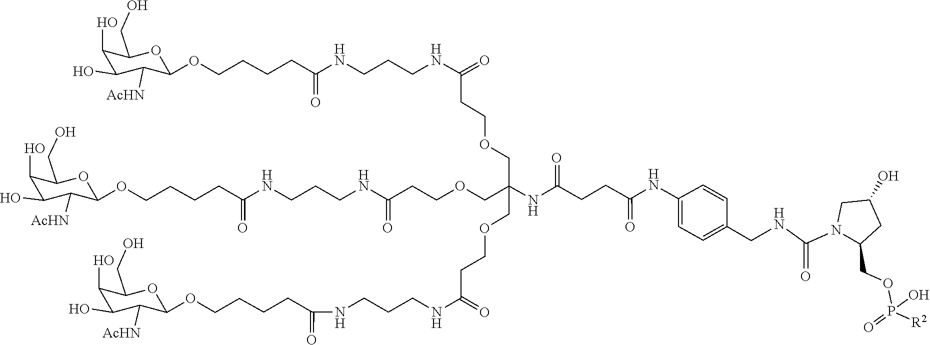
wherein R² is an oligonucleotide comprising or consisting of the sequence set forth in SEQ ID NO: 1/2, 5/6, 147/148, or 179/180;
or a pharmaceutically acceptable salt or ester thereof.

41. The conjugate according to claim 38, wherein the conjugate comprises: 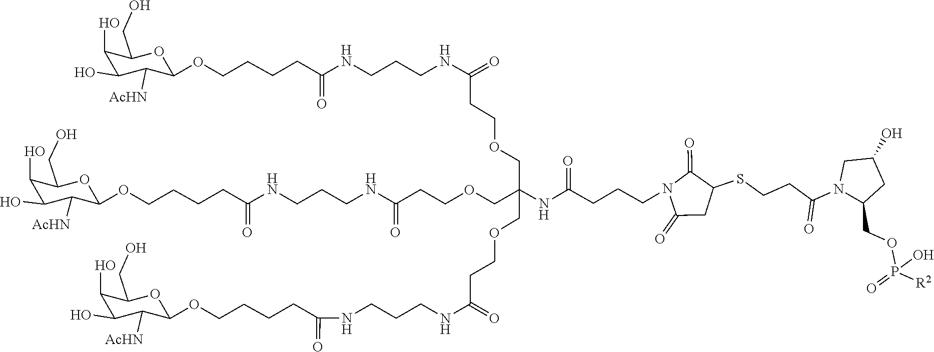
wherein R² is an oligonucleotide comprising or consisting of the sequence set forth in SEQ ID NO: 1/2, 5/6, 147/148, or 179/180;
or a pharmaceutically acceptable salt or ester thereof.

42. The conjugate according to claim 38, wherein the conjugate comprises: 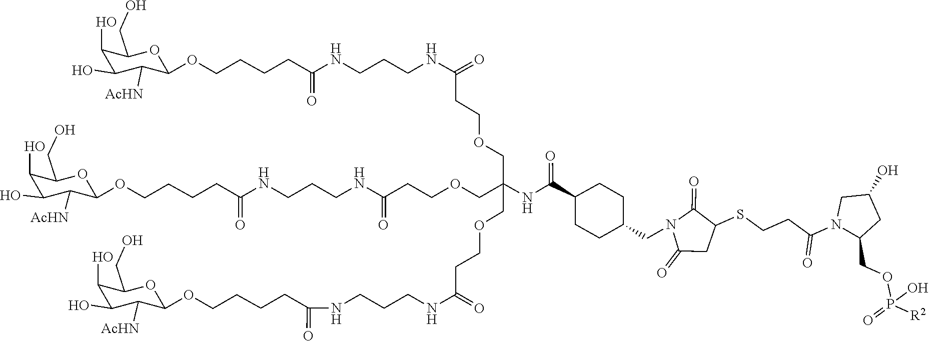
wherein R² is an oligonucleotide comprising or consisting of the sequence set forth in SEQ ID NO: 1/2, 5/6, 147/148, or 179/180;
or a pharmaceutically acceptable salt or ester thereof.

43. The conjugate according to claim 38, wherein the conjugate comprises: 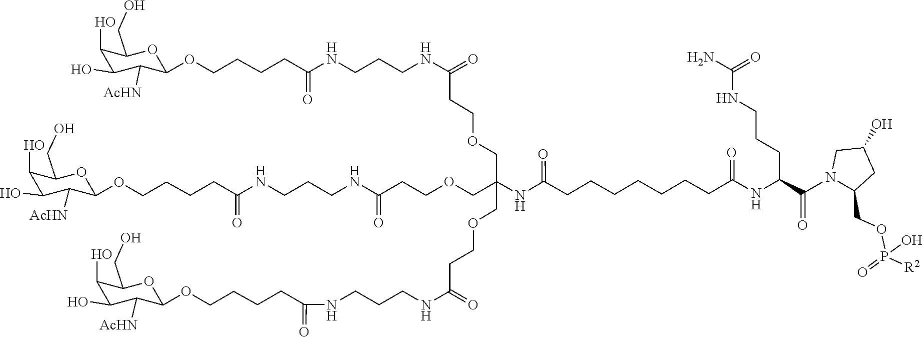
wherein R² is an oligonucleotide comprising or consisting of the sequence set forth in SEQ ID NO: 1/2, 5/6, 147/148, or 179/180;
or a pharmaceutically acceptable salt or ester thereof.

44. A pharmaceutical composition comprising the conjugate of any one of claims 9 to 43, or a pharmaceutically acceptable salt or ester thereof, and a pharmaceutically acceptable carrier.

45. The pharmaceutical composition of claim 44, wherein R² is a PCSK9-siRNA or an ANGPTL3-siRNA.

46. The pharmaceutical composition of claim 44 or 45, wherein the pharmaceutical composition is useful for the treatment and/or prevention of a PCSK9-related disease or disorder.

47. Use of the pharmaceutical composition of any one of claims 44 to 46 for the treatment and/or prevention of a PCSK9-related disease or disorder.

48. Use of the pharmaceutical composition of any one of claims 44 to 46 in the manufacture of a medicament for the treatment and/or prevention of a PCSK9-related disease or disorder.

49. The pharmaceutical composition of claim 46 or the use of claim 47 or 48, wherein the PCSK9-related disease or disorder is atherosclerosis, hypercholesterolemia, acute coronary syndrome, dyslipidemia, myocardial infarction, coronary artery disease, stroke, coronary artery disease, cardiovascular disease, diabetes, hyperlipidemia, type 2 diabetes, or kidney disease.

50. The pharmaceutical composition of claim 44 or 45, wherein the pharmaceutical composition is useful for the treatment and/or prevention of an ANGPTL3-related disease or disorder.

51. Use of the pharmaceutical composition of any one of claims 44, 45 and 50 for the treatment and/or prevention of an ANGPTL3-related disease or disorder.

52. Use of the pharmaceutical composition of any one of claims 44, 45 and 50 in the manufacture of a medicament for the treatment and/or prevention of an ANGPTL3-related disease or disorder.

53. The pharmaceutical composition of claim 50 or the use of claim 51 or 52, wherein the ANGPTL3-related disease or disorder is atherosclerosis, hypercholesterolemia, acute coronary syndrome, dyslipidemia, myocardial infarction, coronary artery disease, stroke, coronary artery disease, cardiovascular disease, diabetes, hyperlipidemia, type 2 diabetes, or kidney disease.

54. The pharmaceutical composition of any one of claims 44 to 46, 50, and 53, wherein the pharmaceutical composition is in a form suitable for parenteral administration.

55. The pharmaceutical composition of claim 54, wherein the parenteral administration is subcutaneous, intramuscular, intravenous, or intraperitoneal administration.

56. The pharmaceutical composition of claim 54 or 55, wherein the pharmaceutical composition is in a form suitable for subcutaneous injection.

57. A method for treating or preventing a PCSK9-related disease or disorder comprising administering a therapeutically effective amount of the conjugate of any one of claims 9 to 43 or a pharmaceutically acceptable salt or ester thereof, or the pharmaceutical composition of any one of claims 44 to 46 and 54 to 56, to a subject in need thereof, such that the PCSK9-related disease or disorder is treated or prevented.

58. The method of claim 57, wherein the PCSK9-related disease or disorder is atherosclerosis, hypercholesterolemia, acute coronary syndrome, dyslipidemia, myocardial infarction, coronary artery disease, stroke, cardiovascular disease, diabetes, hyperlipidemia, type 2 diabetes, or kidney disease.

59. A method for treating or preventing a ANGPTL3-related disease or disorder comprising administering a therapeutically effective amount of the conjugate of any one of claims 9 to 43 or a pharmaceutically acceptable salt or ester thereof, or the pharmaceutical composition of any one of claims 44 to 45, 50, and 54 to 56, to a subject in need thereof, such that the ANGPTL3-related disease or disorder is treated or prevented.

60. The method of claim 59, wherein the ANGPTL3-related disease or disorder is atherosclerosis, hypercholesterolemia, acute coronary syndrome, dyslipidemia, myocardial infarction, coronary artery disease, stroke, coronary artery disease, cardiovascular disease, diabetes, hyperlipidemia, type 2 diabetes, or kidney disease.

61. A method for treating or preventing hypercholesterolemia in a subject in need thereof, the method comprising administering a therapeutically effective amount of the conjugate of any one of claims 9 to 43 or a pharmaceutically acceptable salt or ester thereof, or the pharmaceutical composition of any one of claims 44 to 46, 50, and 54 to 56, to the subject, such that hypercholesterolemia is treated or prevented.

62. The method of claim 61, wherein the hypercholesterolemia is familial hypercholesterolemia, such as heterozygous familial hypercholesterolemia.

63. The method of claim 61, wherein the hypercholesterolemia is non-familial hypercholesterolemia, such as hypercholesterolemia associated with atherosclerotic or cardiovascular disease.

64. A method for treating or preventing atherosclerotic cardiovascular disease in a subject in need thereof, the method comprising administering a therapeutically effective amount of the conjugate of any one of claims 9 to 43 or a pharmaceutically acceptable salt or ester thereof, or the pharmaceutical composition of any one of claims 44 to 46, 50, and 54 to 56, to the subject, such that atherosclerotic cardiovascular disease is treated or prevented.

65. A method for treating or preventing type 2 diabetes in a subject in need thereof, the method comprising administering a therapeutically effective amount of the conjugate of any one of claims 9 to 43 or a pharmaceutically acceptable salt or ester thereof, or the pharmaceutical composition of any one of claims 44 to 46, 50, and 54 to 56, to the subject, such that type 2 diabetes is treated or prevented.

66. The method of any one of claims 57 to 65, wherein the conjugate acts to reduce low-density lipoprotein cholesterol (LDL-C) level in the subject.

67. A method for reducing low-density lipoprotein cholesterol (LDL-C) level in a subject in need thereof, the method comprising administering a therapeutically effective amount of the conjugate of any one of claims 9 to 43 or a pharmaceutically acceptable salt or ester thereof, or the pharmaceutical composition of any one of claims 44 to 46, 50, and 54 to 56, to the subject, such that LDL-C level is reduced in the subject.

68. The method of claim 67, wherein the subject suffers from hypercholesterolemia, dyslipidemia or hyperlipidemia.

69. The method of claim 67 or 68, wherein the subject suffers from atherosclerotic cardiovascular disease.

70. The method of claim 67, wherein the subject suffers from type 2 diabetes.

71. The method of any one of claims 57 to 70, wherein said administering comprises parenteral administration.

72. The method of claim 71, wherein said parenteral administration is subcutaneous, intramuscular, intravenous, or intraperitoneal administration.

73. The method of any one of claims 57 to 72, wherein said administering comprises subcutaneous injection.

74. The method of any one of claims 57 to 73, wherein the subject is a human.

75. The method of any one of claims 57 to 74, wherein the therapeutically effective amount of the conjugate is from about 50 mg to about 500 mg.

76. The method of claim 75, wherein the therapeutically effective amount of the conjugate is from about 200 mg to about 300 mg.

77. The method of claim 75, wherein the therapeutically effective amount of the conjugate is about 50 mg, about 100 mg, about 200 mg, about 300 mg, about 400 mg, or about 500 mg.

78. The method of any one of claims 57 to 77, wherein the conjugate is administered at a dose of about 0.01 mg/kg to about 10 mg/kg, or of about 0.5 mg/kg to about 50 mg/kg, or of about 10 mg/kg to about 30 mg/kg.

79. The method of any one of claims 57 to 78, wherein the conjugate or the composition is administered in two or more doses.

80. The method of claim 79, wherein the conjugate or the composition is administered in a dosing regimen comprising a loading phase followed by a maintenance phase, wherein the loading phase comprises administering a dose of 2 mg/kg, 1 mg/kg or 0.5 mg/kg five times a week, and wherein the maintenance phase comprises administering a dose of 2 mg/kg, 1 mg/kg or 0.5 mg/kg once a week, twice a week, three times a week, once every two weeks, once every three weeks, once a month, once every two months, once every three months, once every four months, once every five months, or once every six months.

81. The method of any one of claims 57 to 80, wherein the conjugate or the composition is administered at intervals of once every about 12 hours, once every about 24 hours, once every about 48 hours, once every about 72 hours, or once every about 96 hours.


SEQUENCE LISTING (238 sequences)

SEQ ID NO: 1 - DNA, 21 bp, Artificial Sequence
  Synthetic oligonucleotide
cuagaccugutuugcuuuugu

SEQ ID NO: 2 - RNA, 23 nt, Artificial sequence
  Synthetic oligonucleotide
acaaaagcaaaacaggucuagaa

SEQ ID NO: 3 - DNA, 21 bp, Artificial Sequence
  Synthetic oligonucleotide
gccuggaguuuauucggaatt

SEQ ID NO: 4 - DNA, 21 bp, Artificial Sequence
  Synthetic oligonucleotide
uuccgaauaaacuccaggctt

SEQ ID NO: 5 - DNA, 21 bp, Artificial Sequence
  Synthetic oligonucleotide
gccuggaguuuauucggaatt

SEQ ID NO: 6 - DNA, 21 bp, Artificial Sequence
  Synthetic oligonucleotide
uuccgaauaaacuccaggctt

SEQ ID NO: 7 - DNA, 20 bp, Artificial Sequence
  Synthetic oligonucleotide
gccuggaguuuauucggatt

SEQ ID NO: 8 - DNA, 21 bp, Artificial Sequence
  Synthetic oligonucletide
uuccgaauaaacuccaggctt

SEQ ID NO: 9 - DNA, 21 bp, Artificial Sequence
  Synthetic oligonucleotide
gccuggaguuuauucggaatt

SEQ ID NO: 10 - DNA, 21 bp, Artificial Sequence
  Synthetic oligonucleotide
uuccgaauaaacuccaggctt

SEQ ID NO: 11 - DNA, 21 bp, Artificial Sequence
  Synthetic oligonucleotide
gccuggaguuuauucggaatt

SEQ ID NO: 12 - DNA, 21 bp, Artificial Sequence
  Synthetic oligonucleotide
uuccgaauaaacuccaggctt

SEQ ID NO: 13 - DNA, 21 bp, Artificial Sequence
  Synthetic oligonucleotide
gccuggaguuuauucggaatt

SEQ ID NO: 14 - DNA, 21 bp, Artificial Sequence
  Synthetic oligonucleotide
uuccgaauaaacuccaggctt

SEQ ID NO: 15 - DNA, 21 bp, Artificial Sequence
  Synthetic oligonucleotide
gccuggaguuuauucggaatt

SEQ ID NO: 16 - DNA, 21 bp, Artificial Sequence
  Synthetic oligonucleotide
uuccgaauaaacuccaggctt

SEQ ID NO: 17 - DNA, 21 bp, Artificial Sequence
  Synthetic oligonucleotide
gccuggaguuuauucggaatt

SEQ ID NO: 18 - DNA, 21 bp, Artificial Sequence
  Synthetic oligonucleotide
gccuggaguuuauucggaatt

SEQ ID NO: 19 - RNA, 21 nt, Artificial Sequence
  Synthetic oligonucleotide
ggaaucuuauauuugauccaa

SEQ ID NO: 20 - RNA, 23 nt, Artificial Sequence
  Synthetic oligonucleotide
uuggaucaaauauaagauucccu

SEQ ID NO: 21 - RNA, 21 nt, Artificial Sequence
  Synthetic oligonucleotide
ggaaucuuauauuugauccaa

SEQ ID NO: 22 - RNA, 23 nt, Artificial Sequence
  Synthetic oligonucleotide
uuggaucaaauauaagauucccu

SEQ ID NO: 23 - RNA, 21 nt, Artificial Sequence
  Synthetic oligonucleotide
ggaaucuuauauuugauccaa

SEQ ID NO: 24 - RNA, 23 nt, Artificial Sequence
  Synthetic oligonucleotide
uuggaucaaauauaagauucccu

SEQ ID NO: 25 - RNA, 21 nt, Artificial Sequence
  Synthetic oligonucleotide
ggaaucuuauauuugauccaa

SEQ ID NO: 26 - RNA, 23 nt, Artificial Sequence
  Synthetic oligonucleotide
uuggaucaaauauaagauucccu

SEQ ID NO: 27 - RNA, 21 nt, Artificial Sequence
  Synthetic oligonucleotide
ggaaucuuauauuugauccaa

SEQ ID NO: 28 - RNA, 23 nt, Artificial Sequence
  Synthetic oligonucleotide
uuggaucaaauauaagauucccu

SEQ ID NO: 29 - RNA, 21 nt, Artificial Sequence
  Synthetic oligonucleotide
ggaaucuuauauuugauccaa

SEQ ID NO: 30 - RNA, 23 nt, Artificial Sequence
  Synthetic oligonucleotide
uuggaucaaauauaagauucccu

SEQ ID NO: 31 - RNA, 21 nt, Artificial Sequence
  Synthetic oligonucleotide
ggaaucuuauauuugauccaa

SEQ ID NO: 32 - RNA, 23 nt, Artificial Sequence
  Synthetic oligonucleotide
uuggaucaaauauaagauucccu

SEQ ID NO: 33 - RNA, 21 nt, Artificial Sequence
  Synthetic oligonucleotide
ggaaucuuauauuugauccaa

SEQ ID NO: 34 - RNA, 23 nt, Artificial Sequence
  Synthetic oligonucleotide
uuggaucaaauauaagauucccu

SEQ ID NO: 35 - RNA, 21 nt, Artificial Sequence
  Synthetic oligonucleotide
ggaaucuuauauuugauccaa

SEQ ID NO: 36 - RNA, 23 nt, Artificial Sequence
  Synthetic oligonucleotide
uuggaucaaauauaagauucccu

SEQ ID NO: 37 - RNA, 21 nt, Artificial Sequence
  Synthetic oligonucleotide
ggaaucuuauauuugauccaa

SEQ ID NO: 38 - RNA, 23 nt, Artificial Sequence
  Synthetic oligonucleotide
uuggaucaaauauaagauucccu

SEQ ID NO: 39 - RNA, 21 nt, Artificial Sequence
  Synthetic oligonucleotide
ggaaucuuauauuugauccaa

SEQ ID NO: 40 - RNA, 23 nt, Artificial Sequence
  Synthetic oligonucleotide
uuggaucaaauauaagauucccu

SEQ ID NO: 41 - RNA, 21 nt, Artificial Sequence
  Synthetic oligonucleotide
ggaaucuuauauuugauccaa

SEQ ID NO: 42 - RNA, 23 nt, Artificial Sequence
  Synthetic oligonucleotide
uuggaucaaauauaagauucccu

SEQ ID NO: 43 - RNA, 21 nt, Artificial Sequence
  Synthetic oligonucleotide
ggaaucuuauauuugauccaa

SEQ ID NO: 44 - RNA, 23 nt, Artificial Sequence
  Synthetic oligonucleotide
uuggaucaaauauaagauucccu

SEQ ID NO: 45 - RNA, 21 nt, Artificial Sequence
  Synthetic oligonucleotide
ggaaucuuauauuugauccaa

SEQ ID NO: 46 - RNA, 23 nt, Artificial Sequence
  Synthetic oligonucleotide
uuggaucaaauauaagauucccu

SEQ ID NO: 47 - RNA, 21 nt, Artificial Sequence
  Synthetic oligonucleotide
ggaaucuuauauuugauccaa

SEQ ID NO: 48 - RNA, 23 nt, Artificial Sequence
  Synthetic oligonucleotide
uuggaucaaauauaagauucccu

SEQ ID NO: 49 - RNA, 21 nt, Artificial Sequence
  Synthetic organism
ggaaucuuauauuugauccaa

SEQ ID NO: 50 - RNA, 23 nt, Artificial Sequence
  Synthetic oligonucleotide
uuggaucaaauauaagauucccu

SEQ ID NO: 51 - RNA, 21 nt, Artificial Sequence
  Synthetic oligonucleotide
ggaaucuuauauuugauccaa

SEQ ID NO: 52 - RNA, 23 nt, Artificial Sequence
  Synthetic oligonucleotide
uuggaucaaauauaagauucccu

SEQ ID NO: 53 - RNA, 21 nt, Artificial Sequence
  Synthetic oligonucleotide
ggaaucuuauauuugauccaa

SEQ ID NO: 54 - RNA, 23 nt, Artificial Sequence
  Synthetic oligonucleotide
uuggaucaaauauaagauucccu

SEQ ID NO: 55 - RNA, 21 nt, Artificial Sequence
  Synthetic oligonucleotide
ggaaucuuauauuugauccaa

SEQ ID NO: 56 - RNA, 23 nt, Artificial Sequence
  Synthetic oligonucleotide
uuggaucaaauauaagauucccu

SEQ ID NO: 57 - RNA, 21 nt, Artificial Sequence
  Synthetic oligonucleotide
ggaaucuuauauuugauccaa

SEQ ID NO: 58 - RNA, 23 nt, Artificial Sequence
  Synthetic oligonucleotide
uuggaucaaauauaagauucccu

SEQ ID NO: 59 - RNA, 21 nt, Artificial Sequence
  Synthetic oligonucleotide
ggaaucuuauauuugauccaa

SEQ ID NO: 60 - RNA, 23 nt, Artificial Sequence
  Synthetic oligonucleotide
uuggaucaaauauaagauucccu

SEQ ID NO: 61 - RNA, 21 nt, Artificial Sequence
  Synthetic oligonucleotide
ggaaucuuauauuugauccaa

SEQ ID NO: 62 - RNA, 23 nt, Artificial Sequence
  Synthetic oligonucleotide
uuggaucaaauauaagauucccu

SEQ ID NO: 63 - RNA, 21 nt, Artificial Sequence
  Synthetic oligonucleotide
ggaaucuuauauuugauccaa

SEQ ID NO: 64 - RNA, 23 nt, Artificial Sequence
  Synthetic oligonucleotide
uuggaucaaauauaagauucccu

SEQ ID NO: 65 - DNA, 21 bp, Artificial Sequence
  Synthetic oligonucleotide
ggaucaucucaagucuuactt

SEQ ID NO: 66 - DNA, 21 bp, Artificial Sequence
  Synthetic oligonucleotide
guaagacuugagaugaucctt

SEQ ID NO: 67 - DNA, 21 bp, Artificial Sequence
  Synthetic oligonucleotide
ggaucaucucaagucuuactt

SEQ ID NO: 68 - DNA, 21 bp, Artificial Sequence
  Synthetic oligonucleotide
guaagacuugagaugaucctt

SEQ ID NO: 69 - DNA, 21 bp, Artificial Sequence
  Synthetic oligonucleotide
ggaucaucucaagucuuactt

SEQ ID NO: 70 - DNA, 21 bp, Artificial Sequence
  Synthetic oligonucleotide
guaagacuugagaugaucctt

SEQ ID NO: 71 - DNA, 21 bp, Artificial Sequence
  Synthetic oligonucleotide
ggaucaucucaagucuuactt

SEQ ID NO: 72 - DNA, 21 bp, Artificial Sequence
  Synthetic oligonucleotide
guaagacuugagaugaucctt

SEQ ID NO: 73 - DNA, 21 bp, Artificial Sequence
  Synthetic oligonucleotide
ggaucaucucaagucuuactt

SEQ ID NO: 74 - DNA, 22 bp, Artificial Sequence
  Synthetic oligonucleotide
guaagacuugagaugauccdtt

SEQ ID NO: 75 - DNA, 21 bp, Artificial Sequence
  Synthetic oligonucleotide
ggaucaucucaagucuuactt

SEQ ID NO: 76 - DNA, 21 bp, Artificial Sequence
  Synthetic oligonucleotide
guaagacuugagaugaucctt

SEQ ID NO: 77 - DNA, 21 bp, Artificial Sequence
  Synthetic oligonucleotide
ggaucaucucaagucuuactt

SEQ ID NO: 78 - DNA, 21 bp, Artificial Sequence
  Synthetic oligonucleotide
guaagacuugagaugaucctt

SEQ ID NO: 79 - DNA, 21 bp, Artificial Sequence
  Synthetic oligonucleotide
ggaucaucucaagucuuactt

SEQ ID NO: 80 - DNA, 21 bp, Artificial Sequence
  Synthetic oligonucleotide
guaagacuugagaugaucctt

SEQ ID NO: 81 - DNA, 21 bp, Artificial Sequence
  Synthetic oligonucleotide
ggaucaucucaagucuuactt

SEQ ID NO: 82 - DNA, 21 bp, Artificial Sequence
  Synthetic oligonucleotide
guaagacuugagaugaucctt

SEQ ID NO: 83 - DNA, 21 bp, Artificial Sequence
  Synthetic oligonucleotide
ggaucaucucaagucuuactt

SEQ ID NO: 84 - DNA, 21 bp, Artificial Sequence
  Synthetic oligonucleotide
guaagacuugagaugaucctt

SEQ ID NO: 85 - DNA, 21 bp, Artificial Sequence
  Synthetic oligonucleotide
ggaucaucucaagucuuactt

SEQ ID NO: 86 - DNA, 21 bp, Artificial Sequence
  Synthetic oligonucleotide
guaagacuugagaugaucctt

SEQ ID NO: 87 - DNA, 21 bp, Artificial Sequence
  Synthetic oligonucleotide
ggaucaucucaagucuuactt

SEQ ID NO: 88 - DNA, 21 bp, Artificial Sequence
  Synthetic oligonucleotide
guaagacuugagaugaucctt

SEQ ID NO: 89 - DNA, 21 bp, Artificial Sequence
  Synthetic oligonucleotide
ggaucaucucaagucuuactt

SEQ ID NO: 90 - DNA, 21 bp, Artificial Sequence
  Synthetic oligonucleotide
guaagacuugagaugaucctt

SEQ ID NO: 91 - DNA, 21 bp, Artificial Sequence
  Synthetic oligonucleotide
ggaucaucucaagucuuactt

SEQ ID NO: 92 - DNA, 21 bp, Artificial Sequence
  Synthetic oligonucleotide
guaagacuugagaugaucctt

SEQ ID NO: 93 - DNA, 21 bp, Artificial Sequence
  Synthetic oligonucleotide
ggaucaucucaagucuuactt

SEQ ID NO: 94 - DNA, 21 bp, Artificial Sequence
  Synthetic oligonucleotide
guaagacuugagaugaucctt

SEQ ID NO: 95 - DNA, 21 bp, Artificial Sequence
  Synthetic oligonucleotdie
ggaucaucucaagucuuactt

SEQ ID NO: 96 - DNA, 21 bp, Artificial Sequence
  Synthetic oligonucleotide
guaagacuugagaugaucctt

SEQ ID NO: 97 - DNA, 21 bp, Artificial Sequence
  Synthetic oligonucleotide
ggaucaucucaagucuuactt

SEQ ID NO: 98 - DNA, 21 bp, Artificial Sequence
  Synthetic oligonucleotide
guaagacuugagaugaucctt

SEQ ID NO: 99 - RNA, 19 nt, Artificial Sequence
  Synthetic oligonucleotide
cugugcuagcaacacccaa

SEQ ID NO: 100 - RNA, 21 nt, Artificial Sequence
  Synthetic oligonucleotide
uuggguguugcuagcacagcc

SEQ ID NO: 101 - RNA, 19 nt, Artificial Sequence
  Synthetic oligonucleotide
cugugcuagcaacacccaa

SEQ ID NO: 102 - RNA, 21 nt, Artificial Sequence
  Synthetic oligonucleotide
uuggguguugcuagcacagcc

SEQ ID NO: 103 - RNA, 19 nt, Artificial Sequence
  Synthetic oligonucleotide
cugugcuagcaacacccaa

SEQ ID NO: 104 - RNA, 21 nt, Artificial Sequence
  Synthetic oligonucleotide
uuggguguugcuagcacagcc

SEQ ID NO: 105 - RNA, 19 nt, Artificial Sequence
  Synthetic oligonucleotide
cugugcuagcaacacccaa

SEQ ID NO: 106 - RNA, 21 nt, Artificial Sequence
  Synthetic oligonucleotide
uuggguguugcuagcacagcc

SEQ ID NO: 107 - RNA, 19 nt, Artificial Sequence
  Synthetic oligonucleotide
cugugcuagcaacacccaa

SEQ ID NO: 108 - RNA, 21 nt, Artificial Sequence
  Synthetic oligonucleotide
uuggguguugcuagcacagcc

SEQ ID NO: 109 - RNA, 19 nt, Artificial Sequence
  Synthetic oligonucleotide
cugugcuagcaacacccaa

SEQ ID NO: 110 - RNA, 21 nt, Artificial Sequence
  Synthetic oligonucleotide
uuggguguugcuagcacagcc

SEQ ID NO: 111 - RNA, 19 nt, Artificial Sequence
  Synthetic oligonucleotide
cugugcuagcaacacccaa

SEQ ID NO: 112 - DNA, 21 bp, Artificial Sequence
  Synthetic oligonucleotide
utggguguugcuagcacagcc

SEQ ID NO: 113 - RNA, 20 nt, Artificial Sequence
  Synthetic oligonucleotide
cugugcuagcdaacacccaa

SEQ ID NO: 114 - RNA, 21 nt, Artificial Sequence
  Synthetic oligonucleotide
uuggguguugcuagcacagcc

SEQ ID NO: 115 - RNA, 19 nt, Artificial Sequence
  Synthetic oligonucleotide
cugugcuagcaacacccaa

SEQ ID NO: 116 - RNA, 21 nt, Artificial Sequence
  Synthetic oligonucleotide
uuggguguugcuagcacagcc

SEQ ID NO: 117 - RNA, 19 nt, Artificial Sequence
  Synthetic oligonucleotide
cugugcuagcaacacccaa

SEQ ID NO: 118 - RNA, 21 nt, Artificial Sequence
  Synthetic oligonucleotide
uuggguguugcuagcacagcc

SEQ ID NO: 119 - RNA, 19 nt, Artificial Sequence
  Synthetic oligonucleotide
cugugcuagcaacacccaa

SEQ ID NO: 120 - RNA, 21 nt, Artificial Sequence
  Synthetic oligonucleotide
uuggguguugcuagcacagcc

SEQ ID NO: 121 - RNA, 19 nt, Artificial Sequence
  Synthetic oligonucleotide
cugugcuagcaacacccaa

SEQ ID NO: 122 - RNA, 21 nt, Artificial Sequence
  Synthetic oligonucleotide
uuggguguugcuagcacagcc

SEQ ID NO: 123 - RNA, 19 nt, Artificial Sequence
  Synthetic oligonucleotide
cugugcuagcaacacccaa

SEQ ID NO: 124 - RNA, 21 nt, Artificial Sequence
  Synthetic oligonucleotide
uuggguguugcuagcacagcc

SEQ ID NO: 125 - RNA, 19 nt, Artificial Sequence
  Synthetic oligonucleotide
cugugcuagcaacacccaa

SEQ ID NO: 126 - RNA, 21 nt, Artificial Sequence
  Synthetic oligonucleotide
uuggguguugcuagcacagcc

SEQ ID NO: 127 - RNA, 19 nt, Artificial Sequence
  Synthetic oligonucleotide
cugugcuagcaacacccaa

SEQ ID NO: 128 - RNA, 21 nt, Artificial Sequence
  Synthetic oligonucleotide
uuggguguugcuagcacagcc

SEQ ID NO: 129 - RNA, 19 nt, Artificial Sequence
  Synthetic oligonucleotide
cugugcuagcaacacccaa

SEQ ID NO: 130 - RNA, 20 nt, Artificial Sequence
  Synthetic oligonucleotide
uugguguugcuagcacagcc

SEQ ID NO: 131 - RNA, 19 nt, Artificial Sequence
  Synthetic oligonucleotide
cugugcuagcaacacccaa

SEQ ID NO: 132 - RNA, 21 nt, Artificial Sequence
  Synthetic oligonucleotide
uuggguguugcuagcacagcc

SEQ ID NO: 133 - RNA, 19 nt, Artificial Sequence
  Synthetic oligonucleotide
cugugcuagcaacacccaa

SEQ ID NO: 134 - RNA, 21 nt, Artificial Sequence
  Synthetic oligonucleotide
uuggguguugcuagcacagcc

SEQ ID NO: 135 - RNA, 19 nt, Artificial Sequence
  Synthetic oligonucleotide
cugugcuagcaacacccaa

SEQ ID NO: 136 - RNA, 21 nt, Artificial Sequence
  Synthetic oligonucleotide
uuggguguugcuagcacagcc

SEQ ID NO: 137 - RNA, 19 nt, Artificial Sequence
  Synthetic oligonucleotide
cugugcuagcaacacccaa

SEQ ID NO: 138 - RNA, 21 nt, Artificial Sequence
  Synthetic oligonucleotide
uuggguguugcuagcacagcc

SEQ ID NO: 139 - RNA, 19 nt, Artificial Sequence
  Synthetic oligonucleotide
cugugcuagcaacacccaa

SEQ ID NO: 140 - RNA, 21 nt, Artificial Sequence
  Synthetic oligonucleotide
uuggguguugcuagcacagcc

SEQ ID NO: 141 - RNA, 19 nt, Artificial Sequence
  Synthetic oligonucleotide
cugugcuagcaacacccaa

SEQ ID NO: 142 - RNA, 21 nt, Artificial Sequence
  Synthetic oligonucleotide
uuggguguugcuagcacagcc

SEQ ID NO: 143 - RNA, 19 nt, Artificial Sequence
  Synthetic oligonucleotide
cugugcuagcaacacccaa

SEQ ID NO: 144 - RNA, 21 nt, Artificial Sequence
  Synthetic oligonucleotide
uuggguguugcuagcacagcc

SEQ ID NO: 145 - RNA, 19 nt, Artificial Sequence
  Synthetic oligonucleotide
cugugcuagcaacacccaa

SEQ ID NO: 146 - RNA, 21 nt, Artificial Sequence
  Synthetic oligonucleotide
uuggguguugcuagcacagcc

SEQ ID NO: 147 - DNA, 21 bp, Artificial Sequence
  Synthetic oligonucleotide
cuagaccugutuugcuuuugu

SEQ ID NO: 148 - RNA, 23 nt, Artificial Sequence
  Synthetic oligonucleotide
acaaaagcaaaacaggucuagaa

SEQ ID NO: 149 - DNA, 21 bp, Artificial Sequence
  Synthetic oligonucleotide
cuagaccugutuugcuuuugu

SEQ ID NO: 150 - RNA, 23 nt, Artificial Sequence
  Synthetic oligonucleotide
acaaaagcaaaacaggucuagaa

SEQ ID NO: 151 - DNA, 21 bp, Artificial Sequence
  Synthetic oligonucleotide
cuagaccugutuugcuuuugu

SEQ ID NO: 152 - RNA, 23 nt, Artificial Sequence
  Synthetic oligonucleotide
acaaaagcaaaacaggucuagaa

SEQ ID NO: 153 - DNA, 21 bp, Artificial Sequence
  Synthetic oligonucleotide
cuagaccugutuugcuuuugu

SEQ ID NO: 154 - RNA, 23 nt, Artificial Sequence
  Synthetic oligonucleotide
acaaaagcaaaacaggucuagaa

SEQ ID NO: 155 - DNA, 21 bp, Artificial Sequence
  Synthetic oligonucleotide
cuagaccugutuugcuuuugu

SEQ ID NO: 156 - RNA, 23 nt, Artificial Sequence
  Synthetic oligonucleotide
acaaaagcaaaacaggucuagaa

SEQ ID NO: 157 - DNA, 21 bp, Artificial Sequence
  Synthetic oligonucleotide
cuagaccugutuugcuuuugu

SEQ ID NO: 158 - RNA, 23 nt, Artificial Sequence
  Synthetic oligonucleotide
acaaaagcaaaacaggucuagaa

SEQ ID NO: 159 - RNA, 21 nt, Artificial Sequence
  Synthetic oligonucleotide
gucucaaaauggaagguuaua

SEQ ID NO: 160 - RNA, 23 nt, Artificial Sequence
  Synthetic oligonucleotide
uauaaccuuccauuuugagacuu

SEQ ID NO: 161 - RNA, 21 nt, Artificial Sequence
  Synthetic oligonucleotide
gucucaaaauggaagguuaua

SEQ ID NO: 162 - RNA, 23 nt, Artificial Sequence
  Synthetic oligonucleotide
uauaaccuuccauuuugagacuu

SEQ ID NO: 163 - RNA, 21 nt, Artificial Sequence
  Synthetic oligonucleotide
gucucaaaauggaagguuaua

SEQ ID NO: 164 - RNA, 23 nt, Artificial Sequence
  Synthetic oligonucleotide
uauaaccuuccauuuugagacuu

SEQ ID NO: 165 - RNA, 21 nt, Artificial Sequence
  Synthetic oligonucleotide
gucucaaaauggaagguuaua

SEQ ID NO: 166 - RNA, 23 nt, Artificial Sequence
  Synthetic oligonucleotide
uauaaccuuccauuuugagacuu

SEQ ID NO: 167 - RNA, 21 nt, Artificial Sequence
  Synthetic oligonucleotide
gucucaaaauggaagguuaua

SEQ ID NO: 168 - RNA, 23 nt, Artificial Sequence
  Synthetic oligonucleotide
uauaaccuuccauuuugagacuu

SEQ ID NO: 169 - RNA, 21 nt, Artificial Sequence
  Synthetic oligonucleotide
gucucaaaauggaagguuaua

SEQ ID NO: 170 - RNA, 23 nt, Artificial Sequence
  Synthetic oligonucleotide
uauaaccuuccauuuugagacuu

SEQ ID NO: 171 - RNA, 21 nt, Artificial Sequence
  Synthetic oligonucleotide
gucucaaaauggaagguuaua

SEQ ID NO: 172 - RNA, 23 nt, Artificial Sequence
  Synthetic oligonucleotide
uauaaccuuccauuuugagacuu

SEQ ID NO: 173 - RNA, 21 nt, Artificial Sequence
  Synthetic oligonucleotide
gucucaaaauggaagguuaua

SEQ ID NO: 174 - RNA, 23 nt, Artificial Sequence
  Synthetic oligonucleotide
uauaaccuuccauuuugagacuu

SEQ ID NO: 175 - RNA, 19 nt, Artificial Sequence
  Synthetic oligonucleotide
aaaucacgaaaccaacuau

SEQ ID NO: 176 - RNA, 21 nt, Artificial Sequence
  Synthetic oligonucleotide
auaguugguuucgugauuucc

SEQ ID NO: 177 - RNA, 19 nt, Artificial Sequence
  Synthetic oligonucleotide
aaaucacgaaaccaacuau

SEQ ID NO: 178 - RNA, 21 nt, Artificial Sequence
  Synthetic oligonucleotide
auaguugguuucgugauuucc

SEQ ID NO: 179 - RNA, 19 nt, Artificial Sequence
  Synthetic oligonucleotide
aaaucacgaaaccaacuau

SEQ ID NO: 180 - RNA, 21 nt, Artificial Sequence
  Synthetic oligonucleotide
auaguugguuucgugauuucc

SEQ ID NO: 181 - RNA, 19 nt, Artificial Sequence
  Synthetic oligonucleotide
aaaucacgaaaccaacuau

SEQ ID NO: 182 - RNA, 21 nt, Artificial Sequence
  Synthetic oligonucleotide
auaguugguuucgugauuucc

SEQ ID NO: 183 - RNA, 19 nt, Artificial Sequence
  Synthetic oligonucleotide
aaaucacgaaaccaacuau

SEQ ID NO: 184 - RNA, 21 nt, Artificial Sequence
  Synthetic oligonucleotide
auaguugguuucgugauuucc

SEQ ID NO: 185 - RNA, 19 nt, Artificial Sequence
  Synthetic oligonucleotide
aaaucacgaaaccaacuau

SEQ ID NO: 186 - RNA, 21 nt, Artificial Sequence
  Synthetic oligonucleotide
auaguugguuucgugauuucc

SEQ ID NO: 187 - RNA, 19 nt, Artificial Sequence
  Synthetic oligonucleotide
aaaucacgaaaccaacuau

SEQ ID NO: 188 - RNA, 21 nt, Artificial Sequence
  Synthetic oligonucleotide
auaguugguuucgugauuucc

SEQ ID NO: 189 - RNA, 19 nt, Artificial Sequence
  Synthetic oligonucleotide
aaaucacgaaaccaacuau

SEQ ID NO: 190 - RNA, 21 nt, Artificial Sequence
  Synthetic oligonucleotide
auaguugguuucgugauuucc

SEQ ID NO: 191 - RNA, 19 nt, Artificial Sequence
  Synthetic oligonucleotide
aaaucacgaaaccaacuau

SEQ ID NO: 192 - RNA, 21 nt, Artificial Sequence
  Synthetic oligonucleotide
auaguugguuucgugauuucc

SEQ ID NO: 193 - RNA, 19 nt, Artificial Sequence
  Synthetic oligonucleotide
aaaucacgaaaccaacuau

SEQ ID NO: 194 - RNA, 21 nt, Artificial Sequence
  Synthetic oligonucleotide
auaguugguuucgugauuucc

SEQ ID NO: 195 - RNA, 19 nt, Artificial Sequence
  Synthetic oligonucleotide
aaaucacgaaaccaacuau

SEQ ID NO: 196 - RNA, 21 nt, Artificial Sequence
  Synthetic oligonucleotide
auaguugguuucgugauuucc

SEQ ID NO: 197 - RNA, 19 nt, Artificial Sequence
  Synthetic oligonucleotide
aaaucacgaaaccaacuau

SEQ ID NO: 198 - RNA, 21 nt, Artificial Sequence
  Synthetic oligonucleotide
auaguugguuucgugauuucc

SEQ ID NO: 199 - RNA, 19 nt, Artificial Sequence
  Synthetic oligonucleotide
aaaucacgaaaccaacuau

SEQ ID NO: 200 - RNA, 21 nt, Artificial Sequence
  Synthetic oligonucleotide
auaguugguuucgugauuucc

SEQ ID NO: 201 - RNA, 19 nt, Artificial Sequence
  Synthetic oligonucleotide
aaaucacgaaaccaacuau

SEQ ID NO: 202 - RNA, 21 nt, Artificial Sequence
  Synthetic oligonucleotide
auaguugguuucgugauuucc

SEQ ID NO: 203 - RNA, 19 nt, Artificial Sequence
  Synthetic oligonucleotide
aaaucacgaaaccaacuau

SEQ ID NO: 204 - RNA, 21 nt, Artificial Sequence
  Synthetic oligonucleotide
auaguugguuucgugauuucc

SEQ ID NO: 205 - RNA, 19 nt, Artificial Sequence
  Synthetic oligonucleotide
aaaucacgaaaccaacuau

SEQ ID NO: 206 - RNA, 21 nt, Artificial Sequence
  Synthetic oligonucleotide
auaguugguuucgugauuucc

SEQ ID NO: 207 - RNA, 19 nt, Artificial Sequence
  Synthetic oligonucleotide
aaaucacgaaaccaacuau

SEQ ID NO: 208 - RNA, 21 nt, Artificial Sequence
  Synthetic oligonucleotide
auaguugguuucgugauuucc

SEQ ID NO: 209 - RNA, 19 nt, Artificial Sequence
  Synthetic oligonucleotide
aaaucacgaaaccaacuau

SEQ ID NO: 210 - RNA, 21 nt, Artificial Sequence
  Synthetic oligonucleotide
auaguugguuucgugauuucc

SEQ ID NO: 211 - RNA, 19 nt, Artificial Sequence
  Synthetic oligonucleotide
aaaucacgaaaccaacuau

SEQ ID NO: 212 - RNA, 21 nt, Artificial Sequence
  Synthetic oligonucleotide
auaguugguuucgugauuucc

SEQ ID NO: 213 - RNA, 19 nt, Artificial Sequence
  Synthetic oligonucleotide
aaaucacgaaaccaacuau

SEQ ID NO: 214 - RNA, 21 nt, Artificial Sequence
  Synthetic oligonucleotide
auaguugguuucgugauuucc

SEQ ID NO: 215 - RNA, 19 nt, Artificial Sequence
  Synthetic oligonucleotide
aaaucacgaaaccaacuau

SEQ ID NO: 216 - RNA, 21 nt, Artificial Sequence
  Synthetic oligonucleotide
auaguugguuucgugauuucc

SEQ ID NO: 217 - RNA, 19 nt, Artificial Sequence
  Synthetic oligonucleotide
aaaucacgaaaccaacuau

SEQ ID NO: 218 - RNA, 21 nt, Artificial Sequence
  Synthetic oligonucleotide
auaguugguuucgugauuucc

SEQ ID NO: 219 - RNA, 19 nt, Artificial Sequence
  Synthetic oligonucleotide
aaaucacgaaaccaacuau

SEQ ID NO: 220 - RNA, 21 nt, Artificial Sequence
  Synthetic oligonucleotide
auaguugguuucgugauuucc

SEQ ID NO: 221 - RNA, 19 nt, Artificial Sequence
  Synthetic oligonucleotide
aaaucacgaaaccaacuau

SEQ ID NO: 222 - RNA, 21 nt, Artificial Sequence
  Synthetic oligonucleotide
auaguugguuucgugauuucc

SEQ ID NO: 223 - RNA, 19 nt, Artificial Sequence
  Synthetic oligonucleotide
aaaucacgaaaccaacuau

SEQ ID NO: 224 - RNA, 21 nt, Artificial Sequence
  Synthetic oligonucleotide
auaguugguuucgugauuucc

SEQ ID NO: 225 - RNA, 21 nt, Artificial Sequence
  Synthetic oligonucleotide
gucucaaaauggaagguuaua

SEQ ID NO: 226 - RNA, 23 nt, Artificial Sequence
  Synthetic oligonucleotide
uauaaccuuccauuuugagacuu

SEQ ID NO: 227 - RNA, 21 nt, Artificial Sequence
  Synthetic oligonucleotide
gucucaaaauggaagguuaua

SEQ ID NO: 228 - RNA, 23 nt, Artificial Sequence
  Synthetic oligonucleotide
uauaaccuuccauuuugagacuu

SEQ ID NO: 229 - RNA, 21 nt, Artificial Sequence
  Synthetic oligonucleotide
gucucaaaauggaagguuaua

SEQ ID NO: 230 - RNA, 23 nt, Artificial Sequence
  Synthetic oligonucleotide
uauaaccuuccauuuugagacuu

SEQ ID NO: 231 - RNA, 21 nt, Artificial Sequence
  Synthetic oligonucleotide
gucucaaaauggaagguuaua

SEQ ID NO: 232 - RNA, 23 nt, Artificial Sequence
  Synthetic oligonucleotide
uauaaccuuccauuuugagacuu

SEQ ID NO: 233 - RNA, 21 nt, Artificial Sequence
  Synthetic oligonucleotide
gucucaaaauggaagguuaua

SEQ ID NO: 234 - RNA, 23 nt, Artificial Sequence
  Synthetic oligonucleotide
uauaaccuuccauuuugagacuu

SEQ ID NO: 235 - RNA, 21 nt, Artificial Sequence
  Synthetic oligonucleotide
gucucaaaauggaagguuaua

SEQ ID NO: 236 - RNA, 23 nt, Artificial Sequence
  Synthetic oligonucleotide
uauaaccuuccauuuugagacuu

SEQ ID NO: 237 - RNA, 21 nt, Artificial Sequence
  Synthetic oligonucleotide
gucucaaaauggaagguuaua

SEQ ID NO: 238 - RNA, 23 nt, Artificial Sequence
  Synthetic oligonucleotide
uauaaccuuccauuuugagacuu